(19) United States
(12) Patent Application Publication
(10) Pub. No.: US 2024/0336681 A1
(43) Pub. Date: Oct. 10, 2024

(54) ANTI-CD47 ANTIBODIES AND USE THEREOF

(71) Applicant: Ludwig-Maximilians-Universität München, Munich (DE)

(72) Inventors: Karl-Peter HOPFNER, Berg/Farchach (DE); Marion SUBKLEWE, Munich (DE); Alexandra SCHELE, Gräfelfing (DE); Nadja FENN, Achmühle (DE); Enrico PERINI, Oberschleissheim (DE); Markus EISELE, Gauting (DE); Elisabeth KREMMER, Munich (DE); Heinrich FLASWINKEL, Munich (DE); Heinrich LEONHARDT, Munich (DE)

(73) Assignee: Ludwig-Maximilians-Universität München, Munich (DE)

(21) Appl. No.: 18/292,001

(22) PCT Filed: Jul. 8, 2022
(86) PCT No.: PCT/EP2022/069177

§ 371 Date: Jan. 25, 2024

(30) Foreign Application Priority Data
Jul. 30, 2021 (SE) 2150980-7

(51) Int. Cl. C07K 16/28; A61K 39/00; A61P 35/00

(52) U.S. Cl. CPC C07K 16/2803; A61P 35/00; A61K 2039/505; C07K 2317/31; C07K 2317/55; C07K 2317/565; C07K 2317/622; C07K 2317/732; C07K 2317/76; C07K 2317/92

(57) ABSTRACT

The present invention relates to an anti-CD47 antibody or an antigen-binding fragment thereof, comprising variable heavy chain complementarity determining regions 1 to 3 (CDRH1, CDRH2, CDRH3) and variable light chain complementarity determining regions 1 to 3 (CDRL1, CDRL2, CDRL3). The anti-CD47 antibody or an antigen-binding fragment thereof is capable of blocking the interaction of CD47 with signal regulatory protein alpha (SIRPalpha). Further envisaged is an anti-CD47 antibody or antigen-binding fragment combined to a further functional component. The invention further relates to a nucleic acid sequence comprising a polynucleotide encoding the anti-CD47 antibody or antigen-binding fragment thereof, a vector comprising the nucleic acid sequence, a host cell comprising the nucleic acid sequence, a method of producing the anti-CD47 antibody or antigen-binding fragment thereof, a product produced by the method as well as a pharmaceutical composition comprising the anti-CD47 antibody or antigen-binding fragment thereof, preferably for use in the treatment of cancer.

### FIELD OF THE INVENTION

The present invention relates to an anti-CD47 antibody or an antigen-binding fragment thereof, comprising variable heavy chain complementarity determining regions 1 to 3 (CDRH1, CDRH2, CDRH3) and variable light chain complementarity determining regions 1 to 3 (CDRL1, CDRL2, CDRL3). The anti-CD47 antibody or an antigen-binding fragment thereof is capable of blocking the interaction of CD47 with signal regulatory protein alpha (SIRPalpha). Further envisaged is an anti-CD47 antibody or antigen-binding fragment combined to a further functional component. The invention further relates to a nucleic acid sequence comprising a polynucleotide encoding the anti-CD47 antibody or antigen-binding fragment thereof, a vector comprising the nucleic acid sequence, a host cell comprising the nucleic acid sequence, a method of producing the anti-CD47 antibody or antigen-binding fragment thereof, a product produced by the method as well as a pharmaceutical composition comprising the anti-CD47 antibody or antigen-binding fragment thereof, preferably for use in the treatment of cancer.

### BACKGROUND OF THE INVENTION

Over the past decades monoclonal antibodies (mABs) and derivatives thereof have become a promising tool of anti-cancer therapies. As of June 2021 the US Food and Drug Administration (FDA) has approved more than 78 mABs for therapeutic use and many more are in clinical development (https://www.antibodysociety.org/resources/approved-antibodies/). Different classes of mABs present various mode of actions. Some specifically bind to a surface antigen on cancer cells and recruit and activate immune effector cells via their fragment crystallizable (Fc) domain to eliminate cancer cells. Others are directed against a surface antigen, however, only antagonistically block a potential receptor-ligand interaction. Recent data suggest that targeting or co-targeting a so-called immune checkpoint is among the most promising approaches against certain types of cancer and can lead to long-term survival of patients (Pardoll et al., 2012 Nature Reviews Cancer; 12:252).

Immune checkpoints are fundamental for the maintenance of self-tolerance under normal physiological conditions and help immune cells to discriminate between “self” and “non-self” (Takahashi et al., 2000, J Exp Med, 192(2):303).

Tumor cells utilize certain immune checkpoints as a main mechanism to escape immune cell recognition and gain immune resistance. Ipilimumab, the first mAB interfering with the immune checkpoint cytotoxic T-lymphocyte-associated antigen 4 (CTLA4) was approved by the FDA in 2011 for the treatment of metastatic melanoma. Further approved mABs target the surface receptor programmed cell death-1 (PD-1) and its ligand programmed cell death ligand-1 (PD-L1). As these are all T cell specific immune checkpoints, both strategies use the adaptive immune system to fight cancer.

However, also within the innate immune system, which acts as the first line of defence against pathogens, there are cellular mechanisms activating immune cells. Typically, these mechanisms are utilized to fight pathogens but they can also be exploited to eliminate cancer cells.

Macrophages are specialized cells (phagocytes) and key players of the innate immune system and link the innate and the adaptive immune response. Macrophages are involved in the detection, engulfment (=phagocytosis) and destruction of cellular debris, bacteria, cancer cells and any other foreign organism that does not display on their surface “markers of self”. Normally these “markers of self” are found on healthy cells and protect these from phagocytosis by macrophages. After engulfment of a foreign cell, macrophages present antigens of this foreign cell (“non-self”) to T cells to induce a T-cell immune response. In addition, macrophages can also initiate inflammation by releasing cytokines that activate other immune cells.

A major regulator of macrophages is the CD47-SIRPalpha (signal regulatory protein alpha, also known as CD172a) signaling pathway. CD47 is broadly expressed as “marker of self” and plays for example a role in the homeostasis of red blood cells (RBCs) and is also found of platelets (PLTs). Its co-receptor SIRPalpha is expressed on myeloid cells such as macrophages. Upon binding to CD47, SIRPalpha conveys a “don't eat me”-signal to macrophages and other phagocytes such as neutrophils, monocytes and dendritic cells (DCs) and thereby negatively regulates phagocytosis. Interestingly, CD47 has been found to be highly expressed on a majority of cancer types to bypass immune cell recognition. CD47 expression often correlates with poor prognosis (Zhao et al., 2018, Transl. Cancer Res; 7(3):609), which led to the development of therapeutic anti-human CD47 (anti-hCD47) mABs throughout the last 8 years.

Anti-hCD47 mABs have been developed to restore immune activation and show anti-tumor effects in various animal models and in patients with hematological malignancies (Zhang et al., 2020, Front. Immunol.; 11:18). However, they can also cause severe toxicities by binding to CD47 on healthy cells, including red blood cells (RBCs) and PLTs. For instance, in many patients treated with anti-hCD47 mABs grade 3 anemia is observed as strong side effect (Advani et al., 2018 N Engl J Med.; 379(18): 1711). Treatments of patients with anti-hCD47 mABs or CD47-targeting molecules resulted in severe thrombocytopenia which can be caused by the aggregation of PLTs or clearance of PLTs by phagoycytosis. Ansell et al., 2021 Clin Cancer Res.; April 15; 27(8):2190-2199). Additionally, a strong systemic binding of CD47 can also lead to severe systemic side effects due to a general reduction in self-tolerance. Even if the side effects could be counteracted by an appropriate treatment, high affinity therapeutic anti-hCD47 antibodies are absorbed by healthy cells due to the ubiquitous expression of CD47. The expression on all cells thus lowers the effective concentration available to bind tumor cells. This phenomenon, called antigen sink, translates into high cost of goods during the production of the antibody, poor pharmacokinetic properties, reduced efficacy and in very high doses needed for clinical applications. In turn, high dosing schemes to overcome the antigen sink will induce even more side effects and toxicities. Because of the ubiquitous expression of CD47 and the high dosing scheme for anti-CD47 antibodies to overcome the antigen sink effect high affinity anti-hCD47 antibodies should not comprise a domain activating a broad range of immune effector cells. Coupling high affinity anti-hCD47 antibodies with a domain that strongly activates immune effector cells such as an IgG1 will direct and activate immune effector cells to eliminate all CD47-expressing cells of the human body. Indeed, in most ongoing clinical trials, high affinity anti-hCD47 antibodies devoid of effector functions are used in combination with tumor targeting antibodies or chemotherapy. The combination of two drugs automatically leads to an even increased risk of side effects, especially when drugs added to the single-agent backbone are targeted small molecules or immunotherapies (Jardim et al, 2019, Oncoimmunlogy 9(1), e1710052).

Thus, there is a clear need for the development of novel and effective anticancer therapies for several cancer indications which allow to exploit the anti-tumor effect of anti-CD47 antibodies while reducing binding to CD47 on healthy cells.

### OBJECTS AND SUMMARY OF THE INVENTION

The present invention addresses this need and provides an anti-CD47 antibody or an antigen-binding fragment thereof, comprising: a variable heavy chain complementarity determining region 1 (CDRH1) sequence selected from the amino acid sequences of SEQ ID NOs: 60 to 62; a variable heavy chain complementarity determining region 2 (CDRH2) sequence selected from the amino acid sequences of SEQ ID NOs: 63 to 73; a variable heavy chain complementarity determining region 3 (CDRH3) sequence of SEQ ID NO: 74; a variable light chain complementarity determining region 1 (CDRL1) sequence selected from the amino acid sequences of SEQ ID NOs: 75 to 79; a variable light chain complementarity determining region 2 (CDRL2) sequence selected from the amino acid sequences of SEQ ID NOs: 80 and 81; and a variable light chain complementarity determining region 3 (CDRL3) sequence selected from the amino acid sequences of SEQ ID NOs: 82 to 85.

The antibodies and antigen-binding fragments thereof according to the invention have been specifically engineered to bind CD47 with low affinity in the range of 100 nanomolar (nM) to 2 micromolar (μM), e. g. in the range of 300 nM to 800 nM. As a comparison, high affinity monoclonal antibodies (mABs) typically have affinities in the low nanomolar (nM) to picomolar (pM) range. For example, the prior art anti-CD47 mAB clone B6H12 binds CD47 with an affinity in the range of 2-5 nM (Zheng et al., 2016, Oncotarget, Vol. 7 (50), 83040), i. e. this standard anti-CD47 mAB is 60 to 400 times more affine to its target CD47, than the antibodies and antigen-binding fragments of the present invention.

The antibodies and antigen-binding fragments thereof according to the invention have been specifically engineered to bind CD47 with a relatively high K_{off} value (fast dissociation) to avoid antigen sink effects by providing weak binding to CD47 while maintaining their anti-tumor effects. Accordingly, the antibodies and antigen-binding fragments thereof of the invention are characterized by fast dissociation kinetics (K_{off} values) in the range of 10⁻² 1/s or more if measured by SPR As a comparison, high affinity monoclonal antibodies targeting CD47 (mABs) typically have K_{off} values in the range of 5×10⁻³ 1/s to 1×10⁻⁵ 1/s. For example, the prior art anti-CD47 mAB clone B6H12 binds CD47 with an K_{off} value of 0.74×10⁻³ 1/s measured by SPR (Zeng et al., 2016, Oncotarget 13; 7(50):83040-83050) and the anti-CD47 mAB CC-90002 with 4.5×10⁻³ 1/s as measured by SPR (Narla et al., 2022, Immunol Immunother 71(2):473-489), i. e. these standard anti-CD47 mABs have an at least 10-50 times lower K_{off} value, than the antibodies and antigen-binding fragments of the present invention.

The present inventors have surprisingly found that despite the low affinity mentioned above, the antibodies and antigen-binding fragments thereof still interfere with CD47 and block the CD47-SIRPalpha interaction. The advantageous effect of this low affinity binding is that only cells that are highly overexpressing CD47, such as cancer cells, are targeted and only on these cells the CD47-SIRPalpha interaction is blocked. This behaviour may additionally be increased if the anti-CD47 antibody or antibody fragment thereof of the present invention is fused to a tumor specific antibody. Even more surprisingly, the antibodies and fragments thereof of the present invention, although having a reduced affinity towards CD47, do not show any increased off-target binding to cells not expressing CD47. Without wishing to be bound by theory, it is assumed that this unexpected behavior can be attributed to the presence of factors providing a good fit between the surfaces of the two molecules (e. g. anti-CD47 antibody and CD47) in their ground state and charge complementarity. These factors are also assumed to yield the high specificity for the target observed.

Due to the low affinity towards CD47, the antibodies and antigen-binding fragments thereof of the present invention show no or significantly reduced binding to CD47 expressed on healthy cells, including RBCs and no signs of PLT aggregation. This leads to the reduction of potential systemic toxicities and unwanted side effects in clinical applications. Furthermore, healthy cells, including RBCs are no longer creating an antigen sink. This in turn advantageously allows for significantly lower treatment doses in clinical applications, thus substantially increasing efficacy and thereby lowering costs of goods. Moreover, as these anti-CD47 antibodies and antigen-binding fragments thereof do not bind to healthy cells, they can be used or combined with an effector cell activation domain such as an IgG1 without risking auto-immunity side effects or PLT aggregation, thus additionally increasing the efficacy of the antibodies of the present invention. The anti-CD47 antibodies and antigen-binding fragments thereof, in addition to the above outlined advantageous properties, allow for a combination with a further functional component relevant to the binding and treatment of cancer cells such as a tumor marker present on the surface of a tumor cell. This combination of functional components may advantageously be either implemented within one protein construct, e. g. as a bi-functional antibody construct according to the present invention comprising an anti-CD47 antibody combined with a further functional component, or in the form of a pharmaceutical composition comprising anti-CD47 antibodies or antigen-binding fragments thereof according to the present invention together with antibodies or antigen-binding fragments thereof targeting specific tumor markers such as Mesothelin (MSLN), CEACAM5, B7H3, CA125, EGFR, Her2 or Mucin-1.

In a preferred embodiment the anti-CD47 antibody or antigen-binding fragment thereof is capable of blocking the interaction of CD47 with signal regulatory protein alpha (SIRPalpha).

In a further preferred embodiment, the antigen-binding fragment of the antibody comprises a variable heavy chain complementarity determining region 1 (CDRH1) sequence selected from the amino acid sequences of SEQ ID Nos: 60, 61 and 62 and a variable heavy chain complementarity determining region 2 (CDRH2) sequence selected from the amino acid sequences of SEQ ID NOs: 63, 64, 65, 66, 67, 68, 69, 70, 71, 72 and 73 and a variable heavy chain complementarity determining region 3 (CDRH3) sequence of SEQ ID NO: 74; and a variable light chain complementarity determining region 1 (CDRL1) sequence selected from the amino acid sequences of SEQ ID NOs: 75, 76, 77, 78 and 79 and a variable light chain complementarity determining region 2 (CDRL2) sequence selected from the amino acid sequences of SEQ ID NOs: 80 and 81 and a variable light chain complementarity determining region 3 (CDRL3) sequence selected from the amino acid sequences of SEQ ID NOs: 82, 83, 84 and 85.

In yet another preferred embodiment said antibody or antigen-binding fragment thereof comprises a variable light chain amino acid sequence selected from the amino acid sequences of SEQ ID NOs: 1 to 28 and a variable heavy chain selected from the amino acid sequences of SEQ ID NOs: 29 to 48.

In a particularly preferred embodiment said antibody or antigen-binding fragment thereof comprises: (i) a variable light chain amino acid sequence of SEQ ID NO: 2 and a variable heavy chain amino acid sequence selected from the amino acid sequences of SEQ ID NOs: 30, 31, 47, or 48; or (ii) a variable light chain amino acid sequence of SEQ ID NO: 3 and a variable heavy chain amino acid sequence selected from the amino acid sequences of SEQ ID NOs: 30, 31, 32, 33, 34, 35, 36, 37, 38, 39, 47, and 48; or (iii) a variable light chain amino acid sequence of SEQ ID NO: 4 and a variable heavy chain amino acid sequence selected from the amino acid sequences of SEQ ID NOs: 30, 31, 32, 33, 34, 35, 36, 37, 38, 39, 47 and 48; or (iv) a variable light chain amino acid sequence of SEQ ID NO: 5 and a variable heavy chain amino acid sequence of SEQ ID NO: 31; or (v) a variable light chain amino acid sequence of SEQ ID NO: 6 and a variable heavy chain amino acid sequence selected from the amino acid sequences of SEQ ID NOs: 31 and 36; or (vi) a variable light chain amino acid sequence of SEQ ID NO: 7 and a variable heavy chain amino acid sequence of SEQ ID NO: 31; or (vii) a variable light chain amino acid sequence of SEQ ID NO: 8 and a variable heavy chain amino acid sequence of SEQ ID NO: 31; or (viii) a variable light chain amino acid sequence of SEQ ID NO: 9 and a variable heavy chain amino acid sequence of SEQ ID NO: 31 and 38; or (ix) a variable light chain amino acid sequence of SEQ ID NO: 10 and a variable heavy chain amino acid sequence selected from the amino acid sequences of SEQ ID NOs: 31, 34, 40, and 41; or (x) a variable light chain amino acid sequence of SEQ ID NO: 11 and a variable heavy chain amino acid sequence of SEQ ID NO: 31; or (xi) a variable light chain amino acid sequence of SEQ ID NO: 12 and a variable heavy chain amino acid sequence of SEQ ID NO: 31; or (xii) a variable light chain amino acid sequence of SEQ ID NO: 13 and a variable heavy chain amino acid sequence of SEQ ID NO: 31; or (xiii) a variable light chain amino acid sequence of SEQ ID NO: 14 and a variable heavy chain amino acid sequence selected from the amino acid sequences of SEQ ID NOs: 31, 34, 40, and 41; or (xiv) a variable light chain amino acid sequence of SEQ ID NO: 15 and a variable heavy chain amino acid sequence selected from the amino acid sequences of SEQ ID NOs: 42, 43, and 46; or (xv) a variable light chain amino acid sequence of SEQ ID NO: 16 and a variable heavy chain amino acid sequence selected from the amino acid sequences of SEQ ID NOs: 42 and 44; or (xvi) a variable light chain amino acid sequence of SEQ ID NO: 17 and a variable heavy chain amino acid sequence of SEQ ID NO: 45; or (xvii) a variable light chain amino acid sequence of SEQ ID NO: 18 and a variable heavy chain amino acid sequence of SEQ ID NO: 46; or (xviii) a variable light chain amino acid sequence of SEQ ID NO: 19 and a variable heavy chain amino acid sequence of SEQ ID NO: 41; or (xix) a variable light chain amino acid sequence of SEQ ID NO: 20 and a variable heavy chain amino acid sequence of SEQ ID NO: 42; or (xx) a variable light chain amino acid sequence of SEQ ID NO: 21 and a variable heavy chain amino acid sequence of SEQ ID NO: 45; or (xxi) a variable light chain amino acid sequence of SEQ ID NO: 22 and a variable heavy chain amino acid sequence selected from the amino acid sequences of SEQ ID NOs: 42 and 46; or (xxii) a variable light chain amino acid sequence of SEQ ID NO: 23 and a variable heavy chain amino acid sequence of SEQ ID NO: 41; or (xxiii) a variable light chain amino acid sequence of SEQ ID NO: 24 and a variable heavy chain amino acid sequence of SEQ ID NO: 41; or (xxiv) a variable light chain amino acid sequence of SEQ ID NO: 25 and a variable heavy chain amino acid sequence of SEQ ID NOs: 42; or (xxv) a variable light chain amino acid sequence of SEQ ID NO: 26 and a variable heavy chain amino acid sequence of SEQ ID NO: 46; or (xxvi) a variable light chain amino acid sequence of SEQ ID NO: 27 and a variable heavy chain amino acid sequence selected from the amino acid sequences of SEQ ID NOs: 30, 31, 47 and 48; or (xxvii) a variable light chain amino acid sequence of SEQ ID NO: 28 and a variable heavy chain amino acid sequence selected from the amino acid sequences of SEQ ID NOs: 30, 31, 47, and 48.

In another preferred embodiment said antibody or antigen-binding fragment thereof comprises a variable light chain amino acid sequence of SEQ ID NO: 15 and a variable heavy chain amino acid sequence of SEQ ID NO: 42.

In a further preferred embodiment said antibody is an IgG1 antibody comprising a constant light (CL) domain and a constant heavy chain CH1, CH2 and CH3 domain, preferably a human constant light (CL) and a human constant heavy chain CH1, CH2, CH3 domain; and a hinge domain, preferably a human hinge region.

It is particularly preferred that said light chain CL domain of the IgG1 antibody comprises an amino acid sequence which is at least 95%, 96%, 97%, 98% or 99% identical to the amino acid sequence of SEQ ID NO: 59.

It is further particularly preferred that said heavy chain CH1 domain of the IgG1 antibody comprises an amino acid sequence which is at least 95%, 96%, 97%, 98% or 99% identical to the amino acid sequence of SEQ ID NO: 49.

It is further particularly preferred that said CH2 domain of the IgG1 antibody comprises an amino acid sequence which is at least 95%, 96%, 97%, 98% or 99% identical to the amino acid sequence of SEQ ID NO: 50.

It is further particularly preferred that said CH3 domain of the IgG1 antibody comprises an amino acid sequence which is at least 95%, 96%, 97%, 98% or 99% identical to the amino acid sequence of SEQ ID NO: 51.

In a further preferred embodiment, said CH2 domain and said CH3 domain of the IgG1 antibody form an Fc domain. It is particularly preferred that said Fc domain comprises an amino acid sequence which is at least 95%, 96%, 97%, 98% or 99% identical to the amino acid sequence of SEQ ID NO: 96.

In a further preferred embodiment said hinge domain of the IgG1 antibody comprises the amino acid sequence of SEQ ID NO: 52.

In yet another preferred embodiment said antibody is an IgG4 antibody comprising a constant light (CL) domain and a constant heavy chain CH1, CH2 and CH3 domain, preferably a human constant light (CL) domain and a human constant heavy chain CH1, CH2, CH3 domain; and a hinge domain, preferably a human hinge region.

It is particularly preferred that said light chain CL domain of the IgG4 antibody comprises an amino acid sequence which is at least 95%, 96%, 97%, 98% or 99% identical to the amino acid sequence of SEQ ID NO: 59.

It is further particularly preferred that said heavy chain CH1 domain of the IgG4 antibody comprises an amino acid sequence which is at least 95%, 96%, 97%, 98% or 99% identical to the amino acid sequence of SEQ ID NO: 54.

It is further particularly preferred that said CH2 domain of the IgG4 antibody comprises an amino acid sequence which is at least 95%, 96%, 97%, 98% or 99% identical to the amino acid sequence of SEQ ID NO: 55.

It is further particularly preferred that said CH3 domain of the IgG4 antibody comprises an amino acid sequence which is at least 95%, 96%, 97%, 98% or 99% identical to the amino acid sequence of SEQ ID NO: 56.

In a further preferred embodiment said CH2 domain and said CH3 domain of the IgG4 antibody form an Fc domain. It is further particularly preferred that Fc domain comprises an amino acid sequence which is at least 95%, 96%, 97%, 98% or 99% identical to the amino acid sequence of SEQ ID NO: 97.

In a further preferred embodiment said hinge domain of the IgG4 antibody comprises the amino acid sequence of SEQ ID NO: 57.

A further embodiment of the present invention relates to an anti-CD47 antibody or antigen-binding fragment as described herein, wherein said antigen-binding fragment of the anti-CD47 antibody is a Fab domain comprising a variable light chain (VL) amino acid sequence and a variable heavy (VH) chain amino acid sequence as defined herein above in (i) to (xxvii) and a constant light (CL) chain amino acid sequence as defined herein above and a constant heavy CH1 chain amino acid sequence as defined herein above.

In a further embodiment the anti-CD47 antibody or antigen-binding fragment thereof according to the invention is an anti-CD47 scFv comprising: a variable light chain amino acid sequence selected from the amino acid sequences of SEQ ID NOs: 1 to 28 and a variable heavy chain selected from the amino acid sequences of SEQ ID NO: 29 to 48, preferably as defined herein above under (i) to (xxvii).

It is particularly preferred that said anti-CD47 scFv comprises a variable light chain amino acid sequence of SEQ ID NO: 15 and a variable heavy chain amino acid sequence of SEQ ID NO: 42.

It is particularly preferred that said anti-CD47 antibody or antigen-binding fragment has an affinity for CD47 measured by surface plasmon resonance (SPR), in the range of 100 nM to 2 μM, preferably in the range of 300 nM to 800 nM.

In a further preferred embodiment said anti-CD47 antibody or antigen-binding fragment has an affinity to its target CD47 which in comparison to the affinity of antibody B6H12 is lower by a factor of at least 20, preferably at least 40, more preferably at least 60.

In a further preferred embodiment said anti-CD47 antibody or antigen-binding fragment as defined herein does not induce platelet aggregation of more than 20%, preferably at a concentration range of 10 nM to 1000 nM, more preferably at 100 nM when changes in absorbance are calculated to percentage of aggregation by reference to the absorbances of PRP and PPP.

In a further preferred embodiment said anti-CD47 antibody or antigen-binding fragment as defined herein has a K_{off} value for the binding to CD47 of about 10⁻² 1/s to 1.0 1/s, preferably of about 5.0×10⁻² 1/s to 9.0×10⁻¹ 1/s, more preferably of about 5.0×10² 1/s to 6.0×10⁻¹ 1/s if measured by surface plasmon resonance (SPR).

In a further aspect the present invention relates to an anti-CD47 antibody or antigen-binding fragment, wherein the anti-CD47 antibody or antigen-binding fragment is combined to a further functional component. The anti-CD47 antibody or antigen-binding fragment is preferably an anti-CD47 antibody or antigen-binding fragment as defined herein above.

In a preferred embodiment said further functional component is a binding domain for a tumor marker present on the surface of a tumor cell.

It is particularly preferred that said tumor marker is Mesothelin (MSLN), CEACAM5, B7H3, CA125, EGFR, Her2 or Mucin-1.

In a further preferred embodiment said combination with a further functional component is a polypeptide fusion via a polypeptide linker, preferably a polypeptide linker comprising or consisting of 4 to 40 amino acids.

It is particularly preferred that said polypeptide linker comprises, essentially consist of, or consists of the amino acids glycine, alanine, proline, lysine and/or serine, preferably of the amino acids glycine and/or serine.

In a further preferred embodiment said polypeptide linker comprises, essentially consists of, or consists of one or more of the amino acid sequence groups of SEQ ID NOs: 86 to 91.

In a further aspect the present invention relates to a nucleic acid molecule comprising a polynucleotide encoding the anti-CD47 antibody or antigen-binding fragment thereof according to the invention.

In a further aspect the present invention relates to a vector comprising the nucleic acid molecule as described herein.

In yet another aspect the present invention relates to a host cell comprising the nucleic acid molecule according to the invention.

In a further aspect the present invention relates to a host cell that expresses the anti-CD47 antibody or antigen-binding fragment thereof as defined herein.

In a further aspect the present invention relates to a method of producing the anti-CD47 antibody or antigen-binding fragment thereof as described herein comprising the cultivation of a host cell as described herein, thereby expressing said protein construct. Further envisaged is a product produced by the method of producing as disclosed herein.

In a further aspect the present invention relates to a pharmaceutical composition comprising the anti-CD47 antibody or antigen-binding fragment as defined herein, or the product of as defined herein and a pharmaceutically acceptable carrier.

In a preferred embodiment the pharmaceutical composition additionally comprises: (i) an antibody or antigen-binding fragment thereof targeting Mesothelin (MSLN); or (ii) an antibody or antigen-binding fragment thereof targeting CEACAM5; or (iii) an antibody or antigen-binding fragment thereof targeting B7H3; or (iv) an antibody or antigen-binding fragment thereof targeting CA125; or (v) an antibody or antigen-binding fragment thereof targeting Mucin-1; or (vi) an antibody or antigen-binding fragment thereof targeting EGFR; or (vii) an antibody or antigen-binding fragment thereof targeting Her2.

In a particularly preferred embodiment the antibody or antigen-binding fragment thereof targeting Mesothelin (MSLN) is Amatuximab, Anetumab, h7D9.v3, or BMS-986148.

In a particularly preferred embodiment the antibody or antigen-binding fragment thereof targeting CEACAM5 is SAR408377, Labetuzumab, SGM-ch511, or Cergutuzumab.

In a particularly preferred embodiment the antibody or antigen-binding fragment thereof targeting B7H3 is Enoblituzumab, MGC018, Omburtamab or MABX-9001.

In a particularly preferred embodiment the antibody or antigen-binding fragment thereof targeting CA125 is Abagovomab or Oregovomab.

In a particularly preferred embodiment the antibody or antigen-binding fragment thereof targeting Mucin-1 is BTH1704, mAb-AR20.5, C595, TAB004, 1B2, HMFG1, PankoMab, KL-6, 5E5, or GGSK-1/30.

In a particularly preferred embodiment the antibody or antigen-binding fragment thereof targeting EGFR is Cetuximab, Panitumumab, Nimotuzumab, Necitumumab, Depatuxizumab, Futuximab, Imgatuzumab, Matuzumab, GC1118, AMG595, Mab A13, MRG003, AVID100, SHR-A1307, RN765C, ABT-414, ABT-806 or ABBV-321.

In a particularly preferred embodiment the antibody or antigen-binding fragment thereof targeting Her2 is Pertuzumab, Trastuzumab, Margetuximab, MCLA-128, GBR 1302, RC48, DS-8201a, FS-1502, SYD985 or ARX788.

In a particularly preferred embodiment the anti-CD47 antibody or antigen-binding fragment as defined herein, the product or the pharmaceutical composition according to the invention are for use in the treatment of cancer.

In a further aspect, the present invention relates to a method for the treatment of cancer, wherein said method comprises administering to a patient in need thereof the anti-CD47 antibody or antigen-binding fragment according to the invention, the product according to the invention, or the pharmaceutical composition according to the invention.

It is particularly preferred that said cancer is ovarian cancer, ascites, mesothelioma, breast cancer, triple negative breast cancer, pancreatic cancer, pancreatic adenocarcinoma, non-small cell lung cancer (NSCLC), colorectal cancer, endometrial cancer, biliary extrahepatic cancer, lymphoma non-hodgkin lymphoma (NHL), Diffuse large B cell lymphoma (DLBCL) leukemia, acute myeloid leukemia (AML) and myelodysplastic syndrome (MDS).

### BRIEF DESCRIPTION OF THE FIGURES

FIG. 1 shows a schematic drawing of a monoclonal antibody (mAb) and possible antigen-binding fragments thereof according to embodiments of the invention. Examples can be formed by an anti-CD47 IgG1 or anti-CD47 IgG4 (A) containing an Fc domain (C), anti-CD47 Fab₂ (B), anti-CD47 Fab (D), anti-CD47 scFv (E) or anti-CD47 scFv fused to an IgG1 (F). VL: variable light, VH: variable heavy, CH₁₋₃: constant heavy, CL: constant light, CDRs: complementary determining regions; linkers are indicated by black lines, disulfide bonds are shown in dashed lines

FIG. 2 illustrates the structural orientation and domain/fragment arrangement of IgG antibodies and protein constructs according to embodiments of the present invention.

FIG. 3 illustrates the purification of different humanized anti-CD47 IgG4 (2D6-000, 2D6-005, 2D6-006, 2D6-015) or IgG1 (2D6-066, 2D6-067, 2D6-076) antibodies as well as a Fab (fragment antigen binding, 2D6-092), which are shown on SDS PAGE under reducing conditions. MW: molecular weight, PageRuler Unstained Protein Ladder, kDa: kilodalton.

FIG. 4 shows different humanized anti-CD47 IgG4 (2D6-000, 2D6-005, 2D6-006, 2D6-015) or IgG1 (2D6-066, 2D6-067, 2D6-076) antibodies, as well as a Fab (fragment antigen binding, 2D6-092). The antibodies are shown after purification by analytical size exclusion chromatography (aSEC).

FIG. 5 shows histograms of the binding of different humanized anti-CD47 IgG4 (2D6-000, 2D6-005, 2D6-015) or IgG1 antibodies (2D6-067), as well as of a Fab (fragment antigen binding, 2D6-092) to CD47-positive Molm-13 cells measured by flow cytometry.

FIG. 6 shows the binding of 16 different humanized anti-CD47 IgG4 antibodies to CD47-positive Expi293 cells measured by flow cytometry. The ratio of mean fluorescence intensity (MFI) between each anti-CD47 IgG4 antibodies and the isotype control is indicated (MFI ratio).

FIG. 7 shows the binding of 3 different humanized anti-CD47 IgG4 antibodies to recombinant C-terminally His-tagged human CD47 by Surface Plasmon Resonance (SPR) using a Biacore X100 (Cytiva). Kₒₙ, K_{off} and KD values are indicated.

FIG. 8 depicts the binding of 6 different humanized anti-CD47 IgG1 to recombinant C-terminally His-tagged human CD47 by Surface Plasmon Resonance (SPR) using a Biacore X100 (Cytiva). KD values are indicated. Kₒₙ, K_{off} and KD values are indicated.

FIG. 9 shows examples of blocking the CD47-SIRPalpha interaction by humanized anti-CD47 IgG4 (2D6-000, 2D6-005, 2D6-015) or IgG1 (2D6-067, 2D6-076) antibodies, as well as by a Fab fragment (2D6-092) by flow cytometry on CD47-positive Molm-13 cells. Percentage blocking obtained with 500 nM of each anti-CD47 antibody is shown.

FIG. 10 shows examples of blocking the CD47-SIRPalpha interaction by SPR using humanized anti-CD47 IgG4 (2D6-005, 2D6-015) or IgG1 (2D6-067, 2D6-076) antibodies, as well as by a Fab fragment (2D6-092). Curves below the buffer binding curve denote blocking.

FIG. 11 depicts antibody-dependent cellular phagocytosis (ADCP) of CD47-positive HL-60 target cells using humanized anti-CD47 IgG4 (2D6-000, 2D6-005, 2D6-015) or IgG1 (2D6-067, 2D6-076) antibodies, as well as a Fab fragment (2D6-092) and human macrophages as effector cells. An anti-CD47 antibody served as positive control.

FIG. 12 depicts the purification of three different anti-CD47 scFvs fused to a full IgG1 antibody targeting a tumor antigen; in A) on SDS PAGE under reducing conditions (MW: molecular weight, PageRuler Unstained Protein Ladder, kDa: kilodalton); and in B) using analytical size exclusion chromatography (aSEC).

FIG. 13 shows examples of binding measurement of humanized anti-CD47 scFvs fused to a full IgG1 antibody targeting a tumor antigen to recombinant C-terminally His-tagged human CD47 by Surface Plasmon Resonance (SPR) using a Biacore X100 (Cytiva). Kₒₙ, K_{off} and KD values are indicated.

FIG. 14 shows examples of blocking the CD47-SIRPalpha interaction by anti-CD47 scFvs fused to a full IgG1 antibody targeting a tumor antigen by flow cytometry on CD47-positive Molm-13 cells. The anti-CD47 IgG1 2D6-000 is used as a positive control. Percentage blocking obtained with 500 nM of each anti-CD47 antibody construct is indicated

FIG. 15 shows examples of blocking the CD47-SIRPalpha interaction by SPR using anti-CD47 scFv fused to a full IgG1 antibody targeting a tumor antigen (2D6-059, 2D6-089). Curves below the buffer binding curve denote blocking.

FIG. 16 shows antibody-dependent cellular phagocytosis (ADCP) of CD47-positive HL-60 target cells using humanized anti-CD47 IgG4 (2D6-000) as well as anti-CD47 scFvs fused to a full IgG1 antibody targeting a tumor antigen (2D6-056, 2D6-059, 2D6-089) and human macrophages as effector cells. An anti-CD47 antibody served as positive control.

FIG. 17 depicts the purification of different anti-CD47 scFvs fused to a full IgG1 antibody targeting the tumor antigen mesothelin (MSL-247, MSL-248, MSL-274); in A) on SDS-PAGE under reducing conditions (MW: molecular weight, PageRuler Unstained Protein Ladder, kDa: kilodalton); and in B) using analytical size exclusion chromatography (aSEC).

FIG. 18 provides examples of binding of different anti-CD47 scFvs fused to a full IgG1 antibody targeting mesothelin (MSL-247, MSL-248, MSL-274) to CD47 by SPR using a Biacore X100 (Cytiva). Kₒₙ, K_{off} and KD values are indicated.

FIG. 19 shows examples of binding of the anti-CD47 scFvs fused to a full IgG1 antibody targeting mesothelin (MSL-247, MSL-248, MSL-274) to CD47 expressed on Molm-13 cells by flow cytometry. Molm-13 cells express CD47 but no mesothelin.

FIG. 20 shows examples of blocking the CD47-SIRPalpha interaction by anti-CD47 scFvs fused to a full IgG1 antibody targeting mesothelin (MSL-247, MSL-248, MSL-274) by flow cytometry on CD47-positive, mesothelin-negative Molm-13 cells. The anti-CD47 IgG1 2D6-000 is used as a positive control. Percentage blocking obtained with 500 nM of each anti-CD47 antibody construct is shown.

FIG. 21 shows examples of blocking the CD47-SIRPalpha interaction by SPR using anti-CD47 scFv fused to a full IgG1 antibody targeting mesothelin (MSL-248, MSL-274). Curves below the buffer binding curve denote blocking.

FIG. 22 shows antibody-dependent cellular phagocytosis (ADCP) of CD47-positive, mesothelin-negative HL-60 cells using anti-CD47 scFvs fused to a full IgG1 antibody targeting mesothelin (MSL-247, MSL-248, MSL-274) and human macrophages as effector cells. An anti-CD47 antibody and the anti-CD47 IgG4 (2D6-000) served as positive control.

FIG. 23 depicts antibody-dependent cellular phagocytosis (ADCP) of CD47-positive, mesothelin-positive OVCAR-3 tumor cells using anti-CD47 scFvs fused to a full IgG1 antibody targeting mesothelin (MSL-247) and human macrophages as effector cells. An anti-CD47 antibody served as positive control. Data represent mean+/−S.D. of 4 independent experiments.

FIG. 24 shows the binding of different humanized anti-CD47 antibodies and fragments thereof measured by flow cytometry on A) wildtype (WT) CD47-positive OVCAR-3 tumor cells and B) on CD47-negative, CD47 knockout (KO) OVCAR-3 tumor cells. CD47 was knocked out by CRISPR-CAS technology. The ratio of mean fluorescence intensity (MFI) of each anti-CD47 antibody and fragments thereof and the isotype control is indicated.

FIG. 25 illustrates the purification of different humanized anti-CD47 scFvs (2D6-096, 2D6-097) shown on SDS PAGE under reducing conditions. MW: molecular weight, Page Ruler Unstained Protein Ladder, kDa: kilodalton. In: Input, FT: Flowthough, W4: Wash fraction 4, E1-E5: Elution fraction 1-5.

FIG. 26 shows different humanized anti-CD47 scFvs (2D6-096, 2D6-097) after purification by analytical size exclusion chromatography (aSEC).

FIG. 27 provides an example of binding of an anti-CD47 scFv (2D6-096) to CD47 by SPR using a Biacore X100 (Cytiva). Kₒₙ, K_{off} and KD values are indicated.

FIG. 28 provides examples of binding of different anti-CD47 scFvs fused to a full IgG1 antibody targeting mesothelin (MSL-253, MSL-741, MSL-742, MSL-745) to CD47 by SPR using a Biacore X100 (Cytiva). Kₒₙ, K_{off} and KD values are indicated.

FIG. 29 illustrates the purification of different humanized anti-CD47 scFvs fused to an IgG1 antibody targeting mesothelin (MSL-741, MSL-742, MSL-745) shown on SDS PAGE under reducing conditions. MW: molecular weight, PageRuler Unstained Protein Ladder, kDa: kilodalton.

FIG. 30 shows percentage of blocking of the CD47-SIRPalpha interaction by CD47 scFv-anti-MSLN protein constructs (MSL-741, MSL-742, MSL-745, MSL-753) by flow cytometry using CD47-positive and MSLN-positiveSuit-2 MSLN cells. Anti-CD47 serves as a positive control and Isotpye as negative control.

FIG. 31 shows examples of blocking the CD47-SIRPalpha interaction by SPR using anti-CD47 scFv fused to a full IgG1 antibody targeting mesothelin (MSL-741, MSL-742, MSL-745). Curves below the buffer binding curve denote blocking.

FIG. 32 depicts antibody-dependent cellular phagocytosis (ADCP) of CD47-positive and MSLN-positive OVCAR-3 tumor cells using anti-CD47 scFvs fused to different full IgG1 antibody targeting mesothelin (MSL-741, MSL-742, MSL-745) and human macrophages as effector cells. An anti-CD47 antibody served as positive control. Data represent mean+/−S.D. of 4 independent experiments.

FIG. 33 shows the binding of anti-CD47 scFvs fused to full IgG1 antibodies targeting mesothelin (MSL-253, MSL-741, MSL-742, MSL-745) measured by flow cytometry on wildtype (WT) Chinese ovarian hamster (CHO) cells and stably transfected CD47-positive (CD47+) CHO cells. The ratio of mean fluorescence intensity (MFI) of each anti-CD47 antibody and fragments thereof and the isotype control is indicated

FIG. 34 exemplary summarizes the binding behavior to red blood cells (RBCs) of the anti-CD47 scFv fused to an IgG1 antibody targeting mesothelin (MSL-253, MSL-741, MSL-742, MSL-745) in comparison to an anti-CD47 antibody and hIgG1 isotype control.

FIG. 35 illustrates the ability of inducing platelet (PLT) aggregation in vitro by a PLT aggregation assay. PLT aggregation after incubation with a humanized anti-CD47 scFvs fused to a mesothelin IgG1 antibody (MSL-745, 2D6-059) is exemplary shown as percentage of aggregation, measured as absorbance at 595 nm on a TECAN plate reader. Anti-CD47 IgG4 serves as negative and anti-CD47-IgG1, including clone B6H12 as positive control.

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given.

As used in this specification and in the appended claims, the singular forms of “a” and “an” also include the respective plurals unless the context clearly dictates otherwise.

In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” or “essentially consisting of” is considered to be a preferred embodiment of the term “comprising of”. If hereinafter a group is defined to comprise at least a certain number of embodiments, this is meant to also encompass a group which preferably consists of these embodiments only.

Furthermore, the terms “(i)”, “(ii)”, “(iii)” or “(a)”, “(b)”, “(c)”, “(d)”, or “first”, “second”, “third” etc. and the like in the description or in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms relate to steps of a method or use there is no time or time interval coherence between the steps, i. e. the steps may be carried out simultaneously or there may be time intervals of seconds, minutes, hours, days, weeks etc. between such steps, unless otherwise indicated.

It is to be understood that this invention is not limited to the particular methodology, protocols, reagents etc. described herein as these may vary. It is also to be understood that the terminology used herein is for the purpose of describing particular embodiments only and is not intended to limit the scope of the present invention that will be limited only by the appended claims. Unless defined otherwise, all technical and scientific terms used herein have the same meanings as commonly understood by one of ordinary skill in the art.

As has been set out above, the present invention concerns in one aspect an anti-CD47 antibody or an antigen-binding fragment thereof, comprising: a variable heavy chain complementarity determining region 1 (CDRH1) sequence selected from the amino acid sequences of SEQ ID NOs: 60, 61 and 62; a variable heavy chain complementarity determining region 2 (CDRH2) sequence selected from the amino acid sequences of SEQ ID NOs: 63, 64, 65, 66, 67, 68, 69, 70, 71, 72 and 73; a variable heavy chain complementarity determining region 3 (CDRH3) sequence of SEQ ID NO: 74; a variable light chain complementarity determining region 1 (CDRL1) sequence selected from the amino acid sequences of SEQ ID NOs: 75, 76, 77, 78 and 79; a variable light chain complementarity determining region 2 (CDRL2) sequence selected from the amino acid sequences of SEQ ID NOs: 80 and 81; and a variable light chain complementarity determining region 3 (CDRL3) sequence selected from the amino acid sequences of SEQ ID NOs: 82, 83, 84 and 85.

The term “CD47” as used herein relates to a transmembrane polypeptide, which belongs to the immunoglobulin superfamily. CD47 partners with membrane integrins and also binds the ligands thrombospondin-1 (TSP-1) and signal-regulatory protein alpha (SIRPalpha) and signal-regulatory protein gamma (SIRPgamma). CD47 functions as a marker of self and transmits a “don't eat me” signal by binding to SIRPalpha expressed by myeloid cells, macrophages, dendritic cells and neutrophils. In this context the role of CD47 is to prevent the engulfment (phagocytosis) of healthy cells by the mentioned immune cells. CD47 is also involved in a range of additional cellular processes, including apoptosis, proliferation, adhesion, migration and angiogenesis (Sick et al, 2012, Br J Pharmacol; 167(7): 1415). Human CD47 (Cluster of Differentiation 47) is identified by UniProt Q08722 and is also known as integrin associated protein (IAP), OA3 or MER6. Alternative splicing of mRNA encoded by the human CD47 gene yields four isoforms which differ in length: isoform 1 (UniProt: Q08722-1, or OA3-323, SEQ ID NO: 92) has been chosen as the canonical isoform in UniProt and which is used as reference isoform for the numbering of the protein (and the numbering of the other isoforms); isoform 2 (Q08722-2, OA3-293, SEQ ID NO: 93), which lacks amino acids corresponding to positions 293 to 323 of SEQ ID NO: 92; isoform 3 (UniProt: Q08722-3, or OA3-305, SEQ ID NO: 94), which lacks amino acids corresponding to positions 306 to 323 of SEQ ID NO: 92 and comprises sequence changes in amino acids corresponding to positions 304 to 305 of SEQ ID NO: 92; and isoform 4 (UniProt: Q08722-4, or OA3-312, SEQ ID NO: 95) which lacks amino acids corresponding to positions 312 to 323 of SEQ ID NO: 92.

Amino acids corresponding to positions 1 to 18 of SEQ ID NO: 92 have been identified as signal peptide, amino acids corresponding to positions 19 to 323 of SEQ ID NO: 92 are typically considered to constitute the canonical CD47. CD47 is typically N-glycosylated a one or more of positions 23, 34, 50, 73, 111 and 206 of SEQ ID NO: 92.

The structure and function of CD47 is described, for example, in Oldenborg, 2013, ISRN Hematol, Epub January 21.

CD47 has been shown to be ubiquitously expressed in human cells and has been found to be ubiquitously expressed on normal tissue. It has further been found to be highly expressed in a wide range of human cancers including acute myeloid leukemia, non-hodgkin lymphoma, ovarian tumors, breast cancer and melanoma, pancreatic cancer, non-small cell lung cancer (NSCLC) (see also Chao et al., 2012, Curr Opin Immunol, 24(2):225-32; Willingham et al, 2012, PNAS, 109 (17); 6662; Xi Q, et al., 2020, J Immunother Cancer, 8:e000253, Zhao et al., 2016, Sci Rep 6: 29719).

Aberrant CD47 expression has been found in several cancers in different species, which may provide the CD47 in modified form deviating from the above defined sequences. Accordingly, the term “CD47” refers to the CD47 polypeptide from any species, preferably from mammals such as rats, mice, and primates, more preferably from humans. It may further include isoforms, fragments, variants or homologues from any species. It is particularly preferred that CD47 is present at the surface of a cell, more preferably at the surface of a human cell.

As used herein, a “fragment”, “variant” or “homologue” of a protein relates to polypeptide which comprises or consists of an amino acid sequence which has at least 70%, preferably 75%, 80%, 85%, 90%, 91%, 92%, 93%, 94%, 95%, 96%, 97%, 98%, 99% or more amino acid sequence identity to the amino acid sequence of a reference protein (e. g. the isoform of SEQ ID NO: 92). In certain embodiments fragments, variants, isoforms and homologues of a reference protein may be capable of performing one, more or all function(s) performed by the reference protein.

The term “sequence identity” as used herein means that amino acids sequences (or two polynucleotides) are identical (i. e., on a residue-by-residue (or on a nucleotide-by-nucleotide) basis) over the comparison window. A percentage of sequence identity may be calculated by comparing two optimally aligned sequences over the window of comparison, determining the number of positions at which the identical elements occur in both sequences to yield the number of matched positions, dividing the number of matched positions by the total number of positions in the comparison window (i. e., the window size), and multiplying the result by 100 to yield the percentage of sequence identity. For amino acid sequences, sequence identity may preferably be determined by using standard techniques known in the art, including the local sequence identity algorithm of Smith and Waterman, 1981, Adv. Appl. Math. 2:482, the sequence identity alignment algorithm of Needleman and Wunsch, 1970, J. Mol. Biol. 48:443, the search for similarity method of Pearson and Lipman, 1988, Proc. Nat. Acad. Sci. U.S.A. 85:2444, or computerized implementations of these algorithms such as multiple sequence alignment tools Clustal Omega (https://www.ebi.ac.uk/Tools/msa/clustalo/), T-coffee/M-coffee (http://tcoffee.crg.cat/), BLAST (https://blast.ncbi.nlm.nih.gov), FASTA (https://www.ebi.ac.uk/Tools/sss/fasta/) or the like, preferably using the default settings. A further envisaged example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, 1987, J. Mol. Evol. 35:351-360. Another example of a useful algorithm is the BLAST algorithm, described in Altschul et al., 1990, J. Mol. Biol. 215:403-410 or the WU-BLAST-2 program. WU-BLAST-2 uses several search parameters, most of which are set to the default values. An additional useful algorithm is gapped BLAST which uses BLOSUM-62 substitution scores.

An “isoform” as used herein refers to a variant of the reference protein expressed by the same species as the species of the reference protein (e. g. Q08722-1 to Q08722-4 as described above).

A “homologue” as used herein refers to a variant of the reference protein produced in different species as compared to the species of the reference protein, e. g. the human species described herein above. In certain embodiments a homologue also includes an orthologue.

A “fragment” as used herein refers to a portion of the reference protein. A “variant” as used herein refers to a protein having an amino acid sequence comprising one or more amino acid substitutions, insertions, deletions or other modifications relative to the amino acid sequence of the reference protein, but retaining a considerable degree of sequence identity (e. g. at least 70%) to the amino acid sequence of the reference protein, e. g. SEQ ID NO: 92, or in further embodiments of the processed form CD47, which is presented at the surface of a cell. A fragment of a reference protein, e. g. of SEQ ID NO: 92, may be of any length (by number of amino acids). In certain embodiments it may have a length of 30%, 40%, 50%, 75%, 80%, 85%, 90%, 91%, 92% 93%, 94%, 95%, 96%, 97%, 98%, or 99% of the length of the reference protein. It is particularly preferred that the fragment is capable of performing one, more or all function(s) performed by the reference protein. The fragment of a reference protein may, for example, have a length of 10, 20, 30, 40, 50, 60, 70, 80, 90, 100 or 110 amino acids.

The term “antibody” as used herein relates to a protein including at least one or two heavy chain (HC) variable regions (abbreviated as VH), and at least one or two light chain (LC) variable regions (abbreviated as VL). The VH and VL regions can further be subdivided into regions of hypervariability, called “complementarity determining regions”, abbreviated as “CDR”, interspersed with more conserved regions termed “framework regions”, abbreviated as “FR”. Antibodies generally comprise six complementarity-determining regions CDRs; three in the heavy chain variable (VH) region: CDRH1, CDRH2 and CDRH3, and three in the light chain variable (VL) region: CDRL1, CDRL2, and CDRL3. The six CDRs define the paratope of the antibody which is the part of the antibody that binds to the target antigen. The VH region and VL region comprise the framework regions (FR1, FR2, FR3 and FR4) at either side of each CDR, which provide a scaffold for the CDRs to display the CDRs on the surface of the VH and VL region. From N-terminus to C-terminus, VH regions comprise the following structure: N terminus-[HC-FR1]-[CDRH1]-[HC-FR2]-[CDRH2]-[HC-FR3]-[CDRH3]-[HC-FR4]-C terminus; and VL regions comprise the following structure: N terminus-[LC-FR1]-[CDRL1]-[LC-FR2]-[CDRL2]-[LC-FR3]-[CDRL3]-[LC-FR4]-C terminus.

There are several different conventions for defining antibody CDRs and FRs such as Kabat et al., 1991, Sequences of Proteins of Immunological Interest, or Chothia et al., 1987, J. Mol. Biol. 196:901-917. Further information would be known to the skilled person or can be derived from suitable literature sources such as http://www.bioinf.org.uk/abs/info.html#cdrid (Prof. Andrew C. R. Martin's group at UCL bioinf.org.uk) or the IMGT database (Brochet et al, 2008, Nucl. Acids Res. 36, W503-508).

The term “antibody” as used herein generally refers to intact immunoglobulins, e. g. of type IgA, IgG, IgE, IgD, or IgM (as well as subtypes thereof), wherein the light chains of the immunoglobulin may be of type kappa or lambda. The antibody further comprises constant regions such as the light chain constant region or domain CL, and heavy chain constant regions or domains CH1, CH2 and CH3. Further, the antibody may comprise a hinge domain. The antibody molecules can be full-length or can, in certain embodiments, be an antigen-binding fragment or antigen-binding molecule derived from an antibody. In certain embodiments, the present invention specifically envisages the use of antigen-binding fragments of an antibody. An “antigen-binding fragment” of an antibody refers to a molecule which is capable of binding to a target antigen or epitope, but does not have an antibody's full length or may differ from a naturally occurring antibody structure. Antigen-binding fragments or synthetic antibodies or antibody derivatives are recombinant proteins derived from gene engineering. Examples of antigen binding fragments encompass Fv, scFv, Fab, scFab, F(ab′)2, Fab2, diabody formats, triabody formats, triplebody formats, scFv-Fc, minibodies, single domain antibodies (e. g. VHH), different variants for truncated antibodies, bispecific antibodies as long as they are capable of binding to the relevant target molecule(s).

The antigen-binding fragment according to the present invention comprises a moiety or moieties capable of binding to a target antigen(s). In some embodiments, the moiety capable of binding to a target antigen comprises an antibody heavy chain variable region (VH) and an antibody light chain variable region (VL) of an antibody capable of specific binding to the target antigen, or sub-forms thereof.

It is particularly preferred that the anti-CD47 antibody is of isotype IgG1 or IgG4. The IgG antibody class comprises four members IgG1, IgG2, IgG3 and IgG4 which are highly conserved but differ in their constant region which is involved in binding to Fc gamma receptors (FcgRs) and the complement protein C1q. As a result, the different subclasses have different effector functions, both in terms of binding to different FcgR and of triggering FcgR-expressing cells, resulting in phagocytosis or antibody-dependent cell-mediated cytotoxicity, and activating the complement cascade (Vidarsson et al, 2014, Front Immunol 5(16):520).

The term “IgG1” antibody as used herein refers to an antibody of the IgG subclass which differs from other antibodies in this subclass (e. g. IgG2, IgG3, IgG4) by differences the hinge region (15 amino acids long) and in the pattern of FcgR binding. The term “IgG4” antibody as used herein refers to an antibody of the IgG subclass which differs from other antibodies in this subclass (e. g. IgG1, IgG2, IgG3) by differences in the amino acid sequence and in the pattern of of FcgR binding, particularly by the weak binding to FcgRI and by the absent binding to FcgRIIIb.

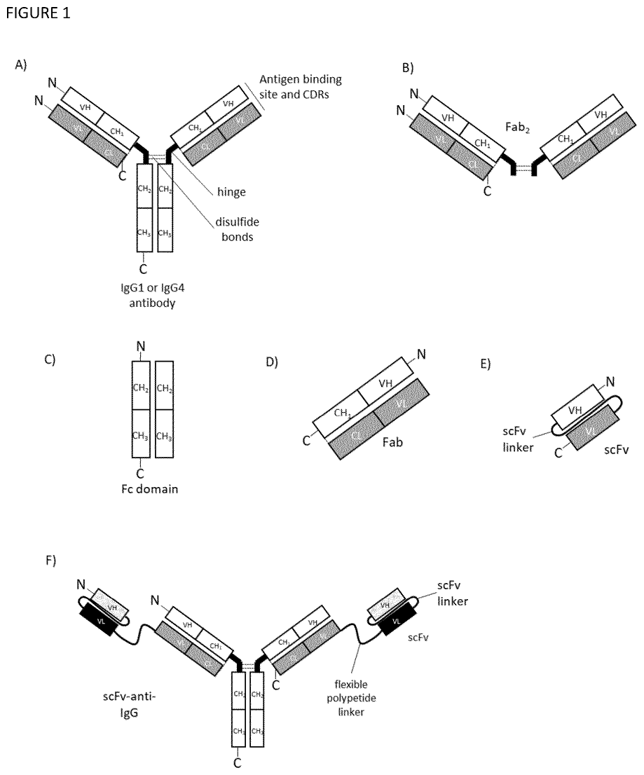

An anti-CD47 antibody, preferably an IgG antibody, more preferably an IgG1 or IgG4 antibody according to the present invention is a glycoprotein of four polypeptide chains of two light chains (LC) and two heavy chains (HC) which are connected by disulphide bonds. The molecular weight of the IgG antibody is ˜150,000 daltons (Da). Each LC consists of two domains, the variable domain (VL) and the constant domain (CL) and has ˜25,000 Da. Two different types of light chains are known, lambda and kappa, where a single anti-CD47 antibody according to the present invention can only contain either lambda or kappa. Each HC has a molecular weight of ˜50,000 Da and comprises a variable (VH) and three constant domains (CH1, CH2 and CH3). The region between CH1 and CH2 is called hinge region. The enzyme papain cleaves the anti-CD47 antibody molecule in the hinge region between the CH1 and CH2 domain. This cleavage results in two identical Fab (fragment antigen binding) fragments, which retain the antigen-binding site (paratope), and one Fc (fragment crystallizable) fragment. The Fc fragment or Fc domain or Fc region is glycosylated and has many different effector functions. E. g. the Fc domain can bind and activate the complement system and can bind and activate FcgRs on macrophages, monocytes or NK cells. Thus, the hinge region connects the two Fab arms to the Fc region. The hinge region allows flexibility between the two Fab domains and the Fc domain to accommodate binding to two antigen binding sites as well as to FcRs. Treatment of an anti-CD47 antibody, preferably an IgG molecule, with pepsin generates the F(ab′)2 fragment, which consists of the two Fab domains linked by the hinge region. Because the F(ab′)2 molecule is bivalent, it is capable of binding two antigens (epitopes). An overview of such molecules is provided in FIG. 1.

The term “Fc region” or “Fc domain” refers to a dimer of heavy chain constant regions CH2 and CH3 which can be linked by disulphide bonds in the hinge region of the antibody. A complete antibody can typically be separated into two Fab regions and one Fc region. The term “Fab region” or “Fab” as used herein comprises a CL and VL domain of the light chain as well as a CH1 and VH domain of the heavy chain of the antibody. The Fc domain of an antibody, i. e. the combination of two CH2 and two CH3 domains, typically interacts with cell surface receptors for Fc (Fc receptors, FcRs) as well as certain proteins of the complement system. IgG FcRs are cell surface molecules situated in the membrane of cells and are expressed by several hematopoietic cells and recognize the Fc region of antibodies and their subclasses. FcRs for IgG are the Fc gamma receptor FcgRI or CD64, FcgRII or CD32, and FcgRIII or CD16. The neonatal FcR (FcRn) expressed on cells of the intestinal epithelium, placenta, and endothelium also binds other IgG type antibodies. Engagement of FcRs expressed by immune cells initiates a number of immune modulatory functions in the immune response. Some FcRs contain activation motifs, i. e. immunoreceptor tyrosine-based activation motif (ITAM) to induce cell signalling such as phagocytosis (antibody-dependent cellular phagocytosis, ADCP), antibody-dependent cellular cytotoxicity (ADCC), complement-dependent cytotoxicity (CDC), degranulation and/or cytokine release, all of which depend on the cell type expressing the FcR and the IgG antibody class and subclass. E. g. engagement of the type I FcRs by immune complexes such as antibodies, induces receptor aggregation followed by immune cell activation. Binding of an IgG antibody to an FcR on an immune effector cell thus often allows not only to recruit the immune cell to the tumor cell, but at the same time also to activate the immune cell by signalling through FcR and thus promoting destruction of the tumor cell.

ADCC is induced when Fc gamma receptors (FcgRs) on innate immune effector cells are engaged by the Fc domain of antibodies that are bound to the surface of the target cells, e. g. to viral proteins on the surface of virus-infected cells or to specific tumor antigens on the surface of tumor cells. This interaction induces the release of cytotoxic granules (containing perforins and granzymes) resulting in killing of infected cells. Multiple innate effector cells, including natural killer (NK) cells, neutrophils, monocytes, and macrophages, are capable of ADCC in vitro. The most important contributors to ADCC in vivo are thought to be NK cells, which express primarily FcgRIIIA. ADCC has been recognized as an important mechanism of action for monoclonal antibodies that target tumor cells.

ADCP or phagocytosis is the uptake of antibody-coated target cells by phagocytic cells. Phagocytic cells, including monocytes, macrophages, neutrophils, eosinophils and dendritic cells (DCs), express Fc receptors such as FcgRI, FcgRIIa, FcgRIIc, FcgRIIIa and Fc alpha RIIIb, which can all mediate immune complex uptake and phagocytosis.

The Fc domain can also induce complement activation, contributing to cell elimination either directly, by means of complement-dependent cytotoxicity (CDC), or indirectly, through phagocytic clearance of complement-coated targets and the induction of an inflammatory response. Activation of the classical complement pathway occurs when the recognition molecule C1q, in complex with the C1r and C1s serine proteases, binds to the Fc domain of an antibody (generally IgG1 and IgM) attached to the cell surface of a target cell. Upon binding of C1q, the proteases C1r and C2r are autocatalytically activated, leading to cleavage of C2 and C4. The larger fragments thereof associate to form C4bC2a on the surface of target cells, and the complex gains the ability to cleave C3 and is termed the C3 convertase The C3 convertase in turn cleaves C3 into C3a (anaphylatoxin) and C3b (Opsonin). C3b can covalently bind to the surface of target cells and tags them as foreign, providing the opsonic signal to phagocytes for ingestion and subsequent killing or degradation. Some of the cleaved C3b remains associated with the C4b2b forming C4b2b3b, the classical pathway C5 convertase. The C5 convertase then cleaves C5 into C5a and C5b. C5b initiates the formation of the pore-forming/membrane attack (MAC) complex, resulting in lysis of the target cell. The release of anaphylatoxins C3a and C5a stimulates a pro-inflammatory environment by inducing the recruitment of immune effector cells and the activation of leukocytes, endothelial cells, epithelial cells, thrombocytes or platelets (Bordron et al, 2020, Clin Rev Allerg Immunolog; 58:155).

The term “anti-CD47 antibody” as used herein particularly refers to an IgG antibody, which specifically binds to CD47 as defined above.

As used herein, an anti-CD47 antibody, e. g. an IgG such as an IgG1 or IgG4 antibody, that “specifically binds” to or is “specific” for a particular polypeptide or an epitope on a particular polypeptide is an antibody that binds to that particular polypeptide or epitope on a particular polypeptide without substantially binding to any other polypeptide or polypeptide epitope with the same domain. In other words, the anti-CD47 antibody according to the present invention is specific for CD47. This does not exclude the possibility that other components, e. g. functional components thereof, including but not limited to those fused or linked to said antibody, bind to different or other polypeptides or epitopes on other polypeptides. For example, such a different component is the Fc domain capable of binding to FcRs, i. e. a component which per se does not bind to CD47, or a binding domain for a tumor marker, e. g. for Mesothelin (MSLN), CEACAM5, B7H3, CA125, EGFR, Her2 or Mucin-1, i. e. a component which per se does not bind to CD47.

Typically, the term “specific binding” or “specifically binds” refers to the ability of an antibody or antigen binding fragment thereof to bind to its target, e. g. CD47 or a tumor marker, with an affinity that is at least five-fold greater than its affinity for a nonspecific antigen. In preferred embodiments an anti-CD47 antibody, e.g. an IgG1 or IgG4 antibody, or an antigen binding fragment thereof such as a scFv or a Fab which specifically binds to CD47, is capable of specifically binding the extracellular domain of CD47 which corresponds to amino acids 19-141 of SEQ ID NO: 92. To block the interaction between SIRPalpha on effector cells and CD47 on tumor cells, the anti-CD47 antibody or antigen binding fragment thereof that specifically binds to CD47 needs to bind a similar or partially overlapping epitope to the binding site for SIRPalpha, in particular to the amino acid positions 19-124 of SEQ ID NO: 92 or parts thereof.

In certain embodiments the anti-CD47 antibody of the present invention is a chimeric antibody. A “chimeric antibody” can be produced by recombinant DNA techniques known in the art. For example, a gene encoding the Fc constant region of a murine (or other species) monoclonal antibody molecule can be digested with restriction enzymes to remove the region encoding the murine Fc, and the equivalent portion of a gene encoding a human Fc constant region may be substituted. A chimeric antibody may also be generated by recombinant DNA techniques where DNA encoding murine variable regions can be ligated to DNA encoding the human constant regions.

In another embodiment, the anti-CD47 antibody of the present invention is a humanized antibody, e. g. humanized by methods known in the art. A humanized antibody consists of non-human CDRs and a framework region and a constant region of a human antibody or derived from a human antibody. For example, a common method for humanization of non-human, i.e. murine or rat antibodies, is CDR grafting. Once murine or rat antibodies are obtained, the CDRs of the non-human antibody are grafted onto the human frameworks. A human framework with high homology to the non-human framework region is selected as acceptor framework for CDR grafting. In other words, humanized antibodies can be generated by replacing sequences of the murine or rat variable region (fragment variable, Fv) that are not directly involved in antigen binding with equivalent sequences from human fragment variable regions. General methods for generating humanized antibodies are known in the art. Accordingly, the present invention envisages antibodies in which specific amino acids have been substituted, deleted, added or back-mutated to the non-human framework. In particular, preferred antibodies may have amino acid substitutions in the framework region, such as to improve, optimize (e. g. increase affinity) or diminish (e. g. decrease affinity) binding to the antigen. For example, a selected, small number of acceptor framework residues of the immunoglobulin chain can be replaced by the corresponding donor amino acids. Preferred locations of the substitutions include amino acid residues adjacent to the CDR, or which are capable of interacting with any of the CDRs. Criteria for selecting amino acids from the donor may, for example, be derived from U.S. Pat. No. 5,585,089. The acceptor framework may, in preferred embodiments, be a mature human antibody framework sequence or a consensus sequence.

It is particularly preferred that the anti-CD47 antibody is a monoclonal antibody. Monoclonal antibodies of defined specificity may be produced using, for instance, the hybridoma technology developed by Köhler and Milstein (Köhler and Milstein, 1976, Eur. J. Immunol., 6: 511-519). Typically, mice or rats are immunized with a recombinant protein. Once an immune response is detected, e. g., antibodies specific for the antigen are detected in the mouse or rat serum, the mouse or rat spleen is harvested and splenocytes isolated. The splenocytes are then fused by well-known techniques to any suitable myeloma cells, for example cells from cell line SP20 or X63AG8.653. Hybridomas are selected and cloned by limited dilution. The hybridoma clones are then assayed by methods known in the art for cells that secrete antibodies capable of binding a polypeptide or protein, such as CD47 disclosed in this invention.

Alternatively, anti-CD47 antibodies according to the present invention can also be generated using various phage or recombinant, synthetic display methods known in the art. In phage display methods, functional antibody domains are displayed on the surface of phage particles, which carry the polynucleotide sequences encoding them. In a particular embodiment, such phages can be utilized to display antigen binding domains expressed from a repertoire or combinatorial antibody library (e. g. human or murine). Phages expressing an antigen binding domain that binds the antigen of interest can be selected or identified with the respective antigen, e. g. using a labeled antigen or an antigen bound or captured to a solid surface or beads. Phages used in these methods are typically filamentous phages including M13. Binding domains expressed from a phage like Fab, Fv or disulfide stabilized Fv antibody domains are recombinantly fused to either the phage gene III or gene VIII protein. Examples of phage display methods that can be used to produce antibodies according to the present invention include those disclosed in Brinkman et al., 1995, J. Immunol. Methods 182: 41-50.

The anti-CD47 antibody, e. g. the IgG1 or IgG4 antibody binding to CD47, or antigen binding fragments thereof or sub-forms of an antibody binding to CD47 described herein may be raised in any mammal, wild-type and/or transgenic, including, for example, mice, rats, rabbits, and goat, or may be produced synthetically, e. g. by expression from vectors, plasmids or artificial chromosomes in suitable host cells.

In specific embodiments, the anti-CD47 antibody or antigen binding fragment thereof which specifically binds to CD47 is specific to a polypeptide comprising the amino acid sequence of SEQ ID NOs: 92 to 95, preferably SEQ ID NO: 92.

According to present invention the anti-CD47 antibody, or antigen binding fragment thereof, which specifically binds to CD47 comprises a VH region comprising the following CDRs:

(1) a CDRH1 sequence selected from the amino acid sequences of SEQ ID NOs: 60 to 62; and (2) a CDRH2 sequence selected from the amino acid sequences of SEQ ID NOs: 63 to 73; and (3) the CDRH3 sequence having the amino acid sequence of SEQ ID NO: 74; or a variant thereof in which one or two or three amino acids in one or more of CDRH1, CDRH2, or CDRH3 are substituted with another amino acid; and comprises a VL region comprising the following CDRs: (4) a CDRL1 sequence selected from the amino acid sequences of SEQ ID NOs: 75 to 79; and (5) a CDRL2 sequence selected from the amino acid sequences of SEQ ID NOs: 80 and 81; and (6) a CDRL3 sequence selected from the amino acid sequences of SEQ ID NOs: 82 to 85, or a variant thereof in which one or two or three amino acids in one or more of CDRL1, CDRL2, or CDRL3 are substituted with another amino acid.

In a preferred embodiment the anti-CD47 antibody or antigen binding fragment thereof, which specifically binds to CD47, comprises a VH region comprising the following CDRs:

(1) the CDRH1 sequence having the amino acid sequence of SEQ ID NO: 60; and (2) the CDRH2 sequence having the amino acid sequence of SEQ ID NO: 71; and (3) the CDRH3 sequence having the amino acid sequence of SEQ ID NO: 74; or a variant thereof in which one or two or three amino acids in one or more of CDRH1, CDRH2, or CDRH3 are substituted with another amino acid; and comprises a VL region comprising the following CDRs: (4) the CDRL1 sequence having the amino acid sequence of SEQ ID NO: 75; and (5) the CDRL2 sequence having the amino acid sequence of SEQ ID NO: 80; and (6) the CDRL3 sequence having the amino acid sequence of SEQ ID NO: 83; or a variant thereof in which one or two or three amino acids in one or more of CDRL1, CDRL2, or CDRL3 are substituted with another amino acid.

The sequences of the CDRH1, 2, 3 and CDRL1, 2, 3 are provided in Tables 1 and 2, respectively. According to the present invention, any CDRH1 sequence mentioned in Table 1 may be combined with any CDRH2 sequence mentioned in Table 1, may be combined with any CDRH3 sequence mentioned in Table 1 for the VH region. According to the present invention, any CDRL1 sequence mentioned in Table 2 may be combined with any CDRL2 sequence mentioned in Table 2 and may be combined with any CDRL3 sequence mentioned in Table 2 for the VL region. Any combination of CDRHs according to Table 1 may be combined with any combination of CDRLs according to Table 2.

TABLE 1

| Name | Sequence | SEQ ID NO |
|---|---|---|
| CDRH1_01 | SYDIN | 60 |
| CDRH1_02 | SYAIN | 61 |
| CDRH1_03 | SYDIS | 62 |
| CDRH2_01 | WIYPRDGSPKYNEKFKG | 63 |
| CDRH2_02 | GIYPRDGSPKYNEKFKG | 64 |
| CDRH2_03 | WIIPRDGSPKYNEKFKG | 65 |
| CDRH2_04 | WIYPRFGSPKYNEKFKG | 66 |
| CDRH2_05 | WIYPRDGSAKYNEKFKG | 67 |
| CDRH2_06 | WIYPRDGSPNYNEKFKG | 68 |
| CDRH2_07 | WIYPRDGSPKYNQKFKG | 69 |
| CDRH2_08 | WIYPRSGSPKYNEKFKG | 70 |
| CDRH2 09 | GIYPRSGSPKYNEKFKG | 71 |
| CDRH2_10 | GIYPRSGSPKYNQKFKG | 72 |
| CDRH2_11 | GIYPRSGSAKYNEKFKG | 73 |
| CDRH3_01 | SVYYGTQFAY | 74 |

TABLE 2

| Name | Sequence | SEQ ID NO |
|---|---|---|
| CDRL1_01 | KSSQSLLNSRTRKNYLA | 75 |
| CDRL1_02 | KSSQSLLYSRTRKNYLA | 76 |
| CDRL1_03 | KSSQSLLNSSTRKNYLA | 77 |
| CDRL1_04 | KSSQSLLNSRNRKNYLA | 78 |
| CDRL1_05 | KSSQSLLNSRTNKNYLA | 79 |
| CDRL2_01 | WASTRDS | 80 |
| CDRL2_02 | WASTRES | 81 |
| CDRL3_01 | KQSFNLVT | 82 |
| CDRL3_02 | QQSFNLVT | 83 |
| CDRL3_03 | KQSFSLVT | 84 |
| CDRL3_04 | KQSFNTVT | 85 |

In a further group of preferred embodiments the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27 or 28.

In a further group of preferred embodiments the anti-CD47 antibody or antigen binding fragment thereof which specifically binds to CD47 comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NOs: 29, 30, 31, 32, 33, 34, 35, 36, 37, 38, 39, 40, 41, 42, 43, 44, 45, 46, 47 or 48.

In a further group of preferred embodiments (i) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 2; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 30, 31, 47 and 48.

In a further group of preferred embodiments (ii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 3; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 30, 31, 32, 33, 34, 35, 36, 37, 38, 39, 47 and 48.

In a further group of preferred embodiments (iii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 4; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 30, 31, 32, 33, 34, 35, 36, 37, 38, 39, 47 and 48.

In a further group of preferred embodiments (iv) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 5; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 31.

In a further group of preferred embodiments (v) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 6; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 31 and 36.

In a further group of preferred embodiments (vi) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 7; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 31.

In a further group of preferred embodiments (vii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 8; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 31.

In a further group of preferred embodiments (viii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 9; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 31 and 38.

In a further group of preferred embodiments (ix) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 10; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 31, 34, 40 and 41.

In a further group of preferred embodiments (x) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 11; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 31.

In a further group of preferred embodiments (xi) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 12; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 31.

In a further group of preferred embodiments (xii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 13; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 31.

In a further group of preferred embodiments (xiii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 14; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 31, 34, 40 and 41.

In a further group of preferred embodiments (xiv) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 15; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 42, 43 and 46.

In a further group of preferred embodiments (xv) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 16; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 42 and 44.

In a further group of preferred embodiments (xvi) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 17; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 45.

In a further group of preferred embodiments (xvii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 18; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 46.

In a further group of preferred embodiments (xviii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 19; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 41.

In a further group of preferred embodiments (xix) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 20; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 42.

In a further group of preferred embodiments (xx) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 21; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 45.

In a further group of preferred embodiments (xxi) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 22; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 42 and 46.

In a further group of preferred embodiments (xxii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 23; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 41.

In a further group of preferred embodiments (xxiii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 24; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 41.

In a further group of preferred embodiments (xxiv) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 25; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid as set forth in SEQ ID NOs: 42.

In a further group of preferred embodiments (xxv) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 26; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence as set forth in SEQ ID NO: 46.

In a further group of preferred embodiments (xxvi) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 27; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 30, 31, 47 and 48.

In a further group of preferred embodiments (xxvii) the anti-CD47 antibody or antigen binding fragment thereof which specifically binds to CD47 comprises a variable light chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable light chain region amino acid sequence as set forth in SEQ ID NO: 28; and comprises a variable heavy chain region amino acid sequence at least about 60%, 65%, 70%, 75%, 80%, 85%, 86%, 87%, 88%, 89%, 90%, 91%, 92%, 93%, 94%, 95%, 96%, 97%, 98%, 99%, or 100% identical to a variable heavy chain region amino acid sequence selected from the amino acid sequences of SEQ ID NOs: 30, 31, 47 and 48.

In an even more preferred embodiment the anti-CD47 antibody or antigen binding fragment thereof comprises a variable light chain region amino acid sequence of SEQ ID NO: 15 and variable heavy chain region amino acid sequence of SEQ ID NO: 42.

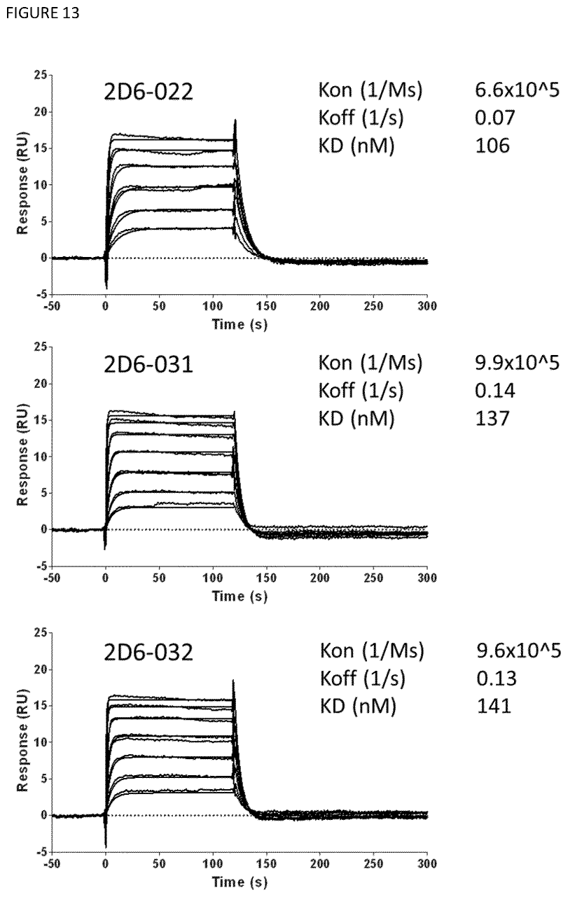

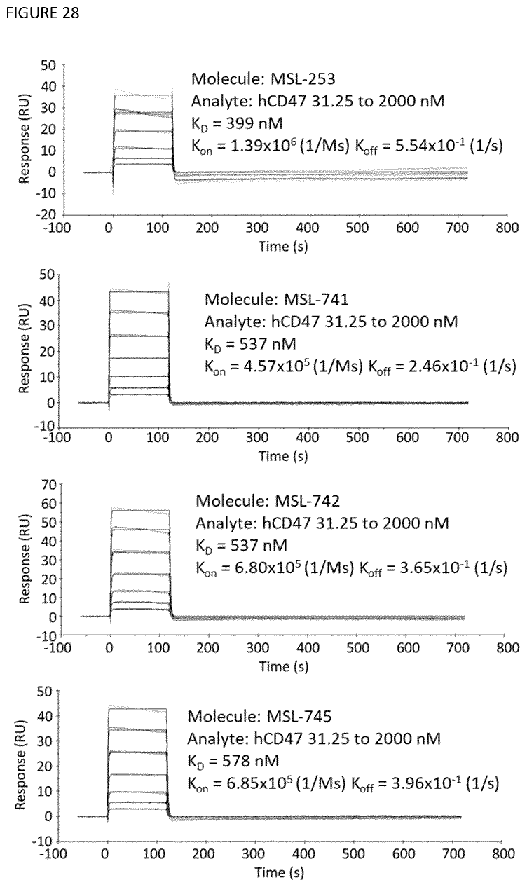

The sequences of the variable light chain region and of the variable heavy chain region of the anti-CD47 antibody or antigen binding fragment thereof and the corresponding antibodies are shown in Table 3. The sequences of the variable light chain region and of the variable heavy chain region of the anti-CD47 antibody or antigen binding fragment thereof according to the invention have been selected based on their overall biochemical and biophysical properties, preferably on the affinity of the anti-CD47 antibody to CD47. The specific VH and VL combinations have been selected as their affinity to CD47 as measured by SPR have been in the suitable and advantageous range of 100-800 nM, e. g. 2D6-022 has an affinity of 106 nM, 2D6-031 has an affinity of 137 nM, 2D6-032 has an affinity of 141 nM, 2D6-046 has an affinity of 406 nM, 2D6-056 has an affinity of 659 nM, 2D6-059 has an affinity of 568 nM, 2D6-088 has an affinity of 460 nM. Also when fused to an anti-MSLN IgG1 antibody, the affinity to CD47 of the respective VH and VL combination are in the range of 100-800 nM, e.g. construct MSL-248 has an affinity of 460 nM, MSL-253 has an affinity of 399 nM, MSL-741 has an affinity of 537 nM and MSL-745 has an affinity of 578 nM. Corresponding measurements are shown in FIG. 13, FIG. 28 and are described in Example 2 (Table 5). In addition, manufacturability, and productivity, developability, immunogenicity, stability and CD47 blocking ability were considered as relevant factors for the selection.

TABLE 3

| | | VH SEQ | | VL SEQ | |
|---|---|---|---|---|---|
| Construct | | | SEQ ID | | SEQ ID |
| name | Format | Name | NO | Name | NO |
| 2D6-000 | chimera: hIgG4 + | VH0 | SEQ ID | VL0 | SEQ ID |
| | mouse VH and VL | | NO: 29 | | NO: 1 |
| | sequences | | | | |
| 2D6-001 | hIgG4 | VH1 | SEQ ID | VL1 | SEQ ID |
| | | | NO: 30 | | NO: 2 |
| 2D6-002 | hIgG4 | VH1 | SEQ ID | VL2 | SEQ ID |
| | | | NO: 30 | | NO: 3 |
| 2D6-003 | hIgG4 | VH1 | SEQ ID | VL3 | SEQ ID |
| | | | NO: 30 | | NO: 27 |
| 2D6-004 | hIgG4 | VH1 | SEQ ID | VL4 | SEQ ID |
| | | | NO: 30 | | NO: 28 |
| 2D6-005 | hIgG4 | VH2 | SEQ ID | VL1 | SEQ ID |
| | | | NO: 31 | | NO: 2 |
| 2D6-006 | hIgG4 | VH2 | SEQ ID | VL2 | SEQ ID |
| | | | NO: 31 | | NO: 3 |
| 2D6-007 | hIgG4 | VH2 | SEQ ID | VL3 | SEQ ID |
| | | | NO: 31 | | NO: 27 |
| 2D6-008 | hIgG4 | VH2 | SEQ ID | VL4 | SEQ ID |
| | | | NO: 31 | | NO: 28 |
| 2D6-009 | hIgG4 | VH3 | SEQ ID | VL1 | SEQ ID |
| | | | NO: 47 | | NO: 2 |
| 2D6-010 | hIgG4 | VH3 | SEQ ID | VL2 | SEQ ID |
| | | | NO: 47 | | NO: 3 |
| 2D6-011 | hIgG4 | VH3 | SEQ ID | VL3 | SEQ ID |
| | | | NO: 47 | | NO: 27 |
| 2D6-012 | hIgG4 | VH3 | SEQ ID | VL4 | SEQ ID |
| | | | NO: 47 | | NO: 28 |
| 2D6-013 | hIgG4 | VH4 | SEQ ID | VL1 | SEQ ID |
| | | | NO: 48 | | NO: 2 |
| 2D6-014 | hIgG4 | VH4 | SEQ ID | VL2 | SEQ ID |
| | | | NO: 48 | | NO: 3 |
| 2D6-015 | hIgG4 | VH4 | SEQ ID | VL3 | SEQ ID |
| | | | NO: 48 | | NO: 27 |
| 2D6-016 | hIgG4 | VH4 | SEQ ID | VL4 | SEQ ID |
| | | | NO: 48 | | NO: 28 |
| 2D6-017 | scFv-hIgG | VH1 | SEQ ID | VL1 | SEQ ID |
| | | | NO: 30 | | NO: 2 |
| 2D6-018 | scFv-hIgG | VH1 | SEQ ID | VL2.1 | SEQ ID |
| | | | NO: 30 | | NO: 4 |
| 2D6-019 | scFv-hIgG | VH1 | SEQ ID | VL3 | SEQ ID |
| | | | NO: 30 | | NO: 27 |
| 2D6-020 | scFv-hIgG | VH1 | SEQ ID | VL4 | SEQ ID |
| | | | NO: 30 | | NO: 28 |
| 2D6-021 | scFv-hIgG | VH2 | SEQ ID | VL1 | SEQ ID |
| | | | NO: 31 | | NO: 2 |
| 2D6-022 | scFv-hIgG | VH2 | SEQ ID | VL2.1 | SEQ ID |
| | | | NO: 31 | | NO: 4 |
| 2D6-023 | scFv-hIgG | VH2 | SEQ ID | VL3 | SEQ ID |
| | | | NO: 31 | | NO: 27 |
| 2D6-024 | scFv-hIgG | VH2 | SEQ ID | VL4 | SEQ ID |
| | | | NO: 31 | | NO: 28 |
| 2D6-025 | scFv-hIgG | VH3 | SEQ ID | VL1 | SEQ ID |
| | | | NO: 47 | | NO: 2 |
| 2D6-026 | scFv-hIgG | VH3 | SEQ ID | VL2.1 | SEQ ID |
| | | | NO: 47 | | NO: 4 |
| 2D6-027 | scFv-hIgG | VH3 | SEQ ID | VL3 | SEQ ID |
| | | | NO: 47 | | NO: 27 |
| 2D6-028 | scFv-hIgG | VH3 | SEQ ID | VL4 | SEQ ID |
| | | | NO: 47 | | NO: 28 |
| 2D6-029 | scFv-hIgG | VH4 | SEQ ID | VL1 | SEQ ID |
| | | | NO: 48 | | NO: 2 |
| 2D6-030 | scFv-hIgG | VH4 | SEQ ID | VL2.1 | SEQ ID |
| | | | NO: 48 | | NO: 4 |
| 2D6-031 | scFv-hIgG | VH4 | SEQ ID | VL3 | SEQ ID |
| | | | NO: 48 | | NO: 27 |
| 2D6-032 | scFv-hIgG | VH4 | SEQ ID | VL4 | SEQ ID |
| | | | NO: 48 | | NO: 28 |
| 2D6-033 | scFv-hIgG | VH2.1 | SEQ ID | VL2.1 | SEQ ID |
| | | | NO: 32 | | NO: 4 |
| 2D6-034 | scFv-hIgG | VH2.2 | SEQ ID | VL2.1 | SEQ ID |
| | | | NO: 33 | | NO: 4 |
| 2D6-035 | scFv-hIgG | VH2.3 | SEQ ID | VL2.1 | SEQ ID |
| | | | NO: 34 | | NO: 4 |
| 2D6-036 | scFv-hIgG | VH2.4 | SEQ ID | VL2.1 | SEQ ID |
| | | | NO: 35 | | NO: 4 |
| 2D6-037 | scFv-hIgG | VH2.5 | SEQ ID | VL2.1 | SEQ ID |
| | | | NO: 36 | | NO: 4 |
| 2D6-038 | scFv-hIgG | VH2.6 | SEQ ID | VL2.1 | SEQ ID |
| | | | NO: 37 | | NO: 4 |
| 2D6-039 | scFv-hIgG | VH2.7 | SEQ ID | VL2.1 | SEQ ID |
| | | | NO: 38 | | NO: 4 |
| 2D6-040 | scFv-hIgG | VH2.8 | SEQ ID | VL2.1 | SEQ ID |
| | | | NO: 39 | | NO: 4 |
| 2D6-041 | scFv-hIgG | VH2 | SEQ ID | VL2.2 | SEQ ID |
| | | | NO: 31 | | NO: 5 |
| 2D6-042 | scFv-hIgG | VH2 | SEQ ID | VL2.3 | SEQ ID |
| | | | NO: 31 | | NO: 6 |
| 2D6-043 | scFv-hIgG | VH2 | SEQ ID | VL2.4 | SEQ ID |
| | | | NO: 31 | | NO: 7 |
| 2D6-044 | scFv-hIgG | VH2 | SEQ ID | VL2.5 | SEQ ID |
| | | | NO: 31 | | NO: 8 |
| 2D6-045 | scFv-hIgG | VH2 | SEQ ID | VL2.6 | SEQ ID |
| | | | NO: 31 | | NO: 9 |
| 2D6-046 | scFv-hIgG | VH2 | SEQ ID | VL2.7 | SEQ ID |
| | | | NO: 31 | | NO: 10 |
| 2D6-047 | scFv-hIgG | VH2 | SEQ ID | VL2.8 | SEQ ID |
| | | | NO: 31 | | NO: 11 |
| 2D6-048 | scFv-hIgG | VH2 | SEQ ID | VL2.9 | SEQ ID |
| | | | NO: 31 | | NO: 12 |
| 2D6-049 | scFv-hIgG | VH2.5 | SEQ ID | VL2.3 | SEQ ID |
| | | | NO: 36 | | NO: 6 |
| 2D6-050 | scFv-hIgG | VH2.7 | SEQ ID | VL2.6 | SEQ ID |
| | | | NO: 38 | | NO: 9 |
| 2D6-051 | scFv-hIgG | VH2 | SEQ ID | VL2.10 | SEQ ID |
| | | | NO: 31 | | NO: 13 |
| 2D6-052 | scFv-hIgG | VH2 | SEQ ID | VL2.11 | SEQ ID |
| | | | NO: 31 | | NO: 14 |
| 2D6-053 | scFv-hIgG | VH2.3 | SEQ ID | VL2.7 | SEQ ID |
| | | | NO: 34 | | NO: 10 |
| 2D6-054 | scFv-hIgG | VH2.9 | SEQ ID | VL2.7 | SEQ ID |
| | | | NO: 40 | | NO: 10 |
| 2D6-055 | scFv-hIgG | VH2.3 | SEQ ID | VL2.11 | SEQ ID |
| | | | NO: 34 | | NO: 14 |
| 2D6-056 | scFv-hIgG | VH2.10 | SEQ ID | VL2.7 | SEQ ID |
| | | | NO: 41 | | NO: 10 |
| 2D6-057 | scFv-hIgG | VH2.9 | SEQ ID | VL2.11 | SEQ ID |
| | | | NO: 40 | | NO: 14 |
| 2D6-058 | scFv-hIgG | VH2.10 | SEQ ID | VL2.11 | SEQ ID |
| | | | NO: 41 | | NO: 14 |
| 2D6-059 | scFv-hIgG | VH2.11 | SEQ ID | VL2.12 | SEQ ID |
| | | | NO: 42 | | NO: 15 |
| 2D6-060 | scFv-hIgG | VH2.12 | SEQ ID | VL2.12 | SEQ ID |
| | | | NO: 43 | | NO: 15 |
| 2D6-061 | scFv-hIgG | VH2.11 | SEQ ID | VL2.13 | SEQ ID |
| | | | NO: 42 | | NO: 16 |
| 2D6-062 | scFv-hIgG | VH2.13 | SEQ ID | VL2.13 | SEQ ID |
| | | | NO: 44 | | NO: 16 |
| 2D6-063 | hIgG1 | VH2.1 | SEQ ID | VL2 | SEQ ID |
| | | | NO: 32 | | NO: 3 |
| 2D6-064 | hIgG1 | VH2.2 | SEQ ID | VL2 | SEQ ID |
| | | | NO: 33 | | NO: 3 |
| 2D6-065 | hIgG1 | VH2.3 | SEQ ID | VL2 | SEQ ID |
| | | | NO: 34 | | NO: 3 |
| 2D6-066 | hIgG1 | VH2.4 | SEQ ID | VL2 | SEQ ID |
| | | | NO: 35 | | NO: 3 |
| 2D6-067 | hIgG1 | VH2.5 | SEQ ID | VL2 | SEQ ID |
| | | | NO: 36 | | NO: 3 |
| 2D6-068 | hIgG1 | VH2.6 | SEQ ID | VL2 | SEQ ID |
| | | | NO: 37 | | NO: 3 |
| 2D6-069 | hIgG1 | VH2.7 | SEQ ID | VL2 | SEQ ID |
| | | | NO: 38 | | NO: 3 |
| 2D6-070 | hIgG1 | VH2.8 | SEQ ID | VL2 | SEQ ID |
| | | | NO: 39 | | NO: 3 |
| 2D6-071 | hIgG1 | VH2 | SEQ ID | VL2.2 | SEQ ID |
| | | | NO: 31 | | NO: 5 |
| 2D6-072 | hIgG1 | VH2 | SEQ ID | VL2.3 | SEQ ID |
| | | | NO: 31 | | NO: 6 |
| 2D6-073 | hIgG1 | VH2 | SEQ ID | VL2.4 | SEQ ID |
| | | | NO: 31 | | NO: 7 |
| 2D6-074 | hIgG1 | VH2 | SEQ ID | VL2.5 | SEQ ID |
| | | | NO: 31 | | NO: 8 |
| 2D6-075 | hIgG1 | VH2 | SEQ ID | VL2.6 | SEQ ID |
| | | | NO: 31 | | NO: 9 |
| 2D6-076 | hIgG1 | VH2 | SEQ ID | VL2.7 | SEQ ID |
| | | | NO: 31 | | NO: 10 |
| 2D6-077 | hIgG1 | VH2 | SEQ ID | VL2.8 | SEQ ID |
| | | | NO: 31 | | NO: 11 |
| 2D6-078 | hIgG1 | VH2 | SEQ ID | VL2.9 | SEQ ID |
| | | | NO: 31 | | NO: 12 |
| 2D6-079 | scFv-hIgG | VH2.14 | SEQ ID | VL2.14 | SEQ ID |
| | | | NO: 45 | | NO: 17 |
| 2D6-080 | scFv-hIgG | VH2.15 | SEQ ID | VL2.15 | SEQ ID |
| | | | NO: 46 | | NO: 26 |
| 2D6-081 | scFv-hIgG | VH2.10 | SEQ ID | VL2.16 | SEQ ID |
| | | | NO: 41 | | NO: 19 |
| 2D6-082 | scFv-hIgG | VH2.11 | SEQ ID | VL2.17 | SEQ ID |
| | | | NO: 42 | | NO: 20 |
| 2D6-083 | scFv-hIgG | VH2.14 | SEQ ID | VL2.18 | SEQ ID |
| | | | NO: 45 | | NO: 21 |
| 2D6-084 | scFv-hIgG | VH2.15 | SEQ ID | VL2.19 | SEQ ID |
| | | | NO: 46 | | NO: 22 |
| 2D6-085 | scFv-hIgG | VH2.10 | SEQ ID | VL2.20 | SEQ ID |
| | | | NO: 41 | | NO: 23 |
| 2D6-086 | scFv-hIgG | VH2.10 | SEQ ID | VL2.21 | SEQ ID |
| | | | NO: 41 | | NO: 24 |
| 2D6-089 | scFv-hIgG | VH2.15 | SEQ ID | VL2.15 | SEQ ID |
| | | | NO: 46 | | NO: 18 |
| 2D6-090 | scFv-hIgG | VH2.15 | SEQ ID | VL2.12 | SEQ ID |
| | | | NO: 46 | | NO: 15 |
| 2D6-091 | scFv-hIgG | VH2.11 | SEQ ID | VL2.19 | SEQ ID |
| | | | NO: 42 | | NO: 22 |
| 2D6-092 | Fab | VH2 | SEQ ID | VL2 | SEQ ID |
| | | | NO: 31 | | NO: 3 |
| 2D6-093 | scFv-hIgG | VH2.15 | SEQ ID | VL2.17 | SEQ ID |
| | | | NO: 46 | | NO: 20 |
| 2D6-094 | IgG1 | VH2.10 | SEQ ID | VL2.11 | SEQ ID |
| | | | NO: 41 | | NO: 14 |
| 2D6-095 | IgG4 | VH2.10 | SEQ ID | VL2.11 | SEQ ID |
| | | | NO: 41 | | NO: 14 |
| 2D6-096 | scFv | VH3 | SEQ ID | VL3 | SEQ ID |
| | | | NO: 47 | | NO: 27 |
| 2D6-097 | scFv | VH3 | SEQ ID | VL4 | SEQ ID |
| | | | NO: 47 | | NO: 28 |
| MSL-247 | scFv-hIgG | VH2.15 | SEQ ID | VL2.15 | SEQ ID |
| | | | NO: 42 | | NO: 25 |
| MSL-248 | scFv-hIgG | VH2.11 | SEQ ID | VL2.12 | SEQ ID |
| | | | NO: 42 | | NO: 15 |
| MSL-274 | scFv-hIgG | VH2.15 | SEQ ID | VL2.15 | SEQ ID |
| | | | NO: 42 | | NO: 15 |
| MSL-253 | scFv-hIgG | VH2.11 | SEQ ID | VL2.12 | SEQ ID |
| | | | NO: 42 | | NO: 15 |
| MSL-741 | scFv-hIgG | VH2.11 | SEQ ID | VL2.12 | SEQ ID |
| | | | NO: 42 | | NO: 15 |
| MSL-742 | scFv-hIgG | VH2.11 | SEQ ID | VL2.12 | SEQ ID |
| | | | NO: 42 | | NO: 15 |
| MSL-745 | scFv-hIgG | VH2.11 | SEQ ID | VL2.12 | SEQ ID |
| | | | NO: 42 | | NO: 15 |

Format explanations: hIgG4: humanized IgG4, VH: variable heavy, VL: variable light, scFv-hIgG: single chain fragment variable fused to human IgG, hIgG1: humanized IgG1; scFv: single chain fragment variable.

In further embodiments said anti-CD47 antibody or antigen binding fragment thereof has a K_{off} value for binding to CD47 of about 10⁻² 1/s or higher if measured by SPR.

In preferred embodiments said anti-CD47 antibody or antigen binding fragment thereof has a K_{off} value for binding to CD47 of about 10⁻² 1/s to 1 1/s, more preferably of about 5×10⁻² 1/s to 9×10⁻¹ 1/s, most preferably of about 5×10⁻² 1/s to 6×10⁻¹ 1/s if measured by SPR.

In an additional group of embodiments the K_{off} value for the binding of said anti-CD47 antibody or antigen binding to CD47 is at least 10, 20, 30, 40 times, preferably 50 times higher if measured by SPR, in comparison to the binding of known anti-CD47 mABs, such as the anti-CD47 antibody clone B6H12 or CC-90002, to CD47.

In further preferred embodiments said anti-CD47 antibody is an IgG1 antibody and comprises a constant light chain domain (CL), e.g. a kappa CL domain and a constant heavy chain (CH) consisting of a CH1, CH2 and CH3 domain. In further preferred embodiments said anti-CD47 antibody is an IgG1 antibody and comprises a human CL domain, e.g. a human kappa CL domain and a human CH chain consisting of CH1, CH2, CH3 domain and a hinge domain. The hinge domain is preferably a human hinge region. The present invention further envisages antigen binding fragments of said IgG1 antibody, which specifically bind to CD47.

In further preferred embodiments the IgG1 anti-CD47 antibody or an antigen binding fragment thereof, which specifically binds to CD47, comprises a constant light chain domain (CL) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 59.

In further preferred embodiments the IgG1 anti-CD47 antibody or an antigen binding fragment thereof, which specifically binds to CD47, comprises a constant heavy chain domain 1 (CH1) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 49.

In further preferred embodiments the IgG1 anti-CD47 antibody or an antigen binding fragment thereof, which specifically binds to CD47, comprises a constant heavy chain domain 2 (CH2) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 50.

In further preferred embodiments the IgG1 anti-CD47 antibody or an antigen binding fragment thereof, which specifically binds to CD47, comprises a constant heavy chain domain 3 (CH3) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 51.

In further particularly preferred embodiments, the IgG1 anti-CD47 antibody comprises a constant heavy chain domain 1 (CH1) comprising an amino acid sequence which is at least about 95%, 96%, 97%, 98%, 99%, or 100% identical to the amino acid sequence of SEQ ID NO: 49; and a constant heavy chain domain 2 (CH2) comprising an amino acid sequence which is at least about 95%, 96%, 97%, 98%, 99%, or 100% identical to the amino acid sequence of SEQ ID NO: 50; and a constant heavy chain domain 3 (CH3) comprising an amino acid sequence which is at least about 95%, 96%, 97%, 98%, 99%, or 100% identical to the amino acid sequence of SEQ ID NO: 51; and a constant light chain domain (CL) comprising an amino acid sequence which is at least about 95%, 96%, 97%, 98%, 99%, or 100% identical to the amino acid sequence of SEQ ID NO: 59. In further specific embodiments, the IgG1 anti-CD47 antibody comprises CDR sequences as defined herein above, or VH and/or VL sequences as defined herein above together with a CL and a CH1 and a CH2 and a CH3 sequence as defined herein above.

In a further embodiment, the CH2 domain as defined above of the IgG1 antibody and the CH3 domain of the IgG1 antibody as defined above form an Fc domain. Preferably, the Fc domain comprises a CH2 domain comprising an amino acid sequence which is at least about 95%, 96%, 97%, 98%, 99%, or 100% identical to the amino acid sequence of SEQ ID NO: 50 and a CH3 domain comprising an amino acid sequence which is at least about 95%, 96%, 97%, 98%, 99%, or 100% identical to the amino acid sequence of SEQ ID NO: 51. It is further preferred that the Fc domain comprises an amino acid sequence which is at least 95%, 96%, 97%, 98% or 99% identical to the amino acid sequence of SEQ ID NO: 96.

In further preferred embodiments the IgG1 anti-CD47 antibody or antigen binding fragment thereof, which specifically binds to CD47 comprises a hinge domain comprising an amino acid sequence which is at least about 95%, 96%, 97%, 98%, 99%, or 100% identical to the amino acid sequence of the amino acid sequence of SEQ ID NO: 52. The term “hinge domain” or alternatively “hinge region” as used herein refers to a functional domain in an antibody, which connects the CH1 and the CH2 domain and thus is between the Fab and Fc domain. It typically comprises two disulphide bonds to dimerize two heavy chains and thereby contributes to the three-dimensional form and structure of an antibody. Its sequence, structure and position provided segmental flexibility to promote the antibody functionality.

In a further embodiment, said CH1, hinge, CH2 and CH3 domain of the IgG1 antibody form the heavy constant chain. It is particularly preferred that said heavy constant chain comprises an amino acid sequence which is at least 95%, 96%, 97%, 98% or 99% identical to the amino acid sequence of SEQ ID NO: 53.

In a further preferred embodiment the IgG1 anti-CD47 antibody or antigen binding fragment thereof, which specifically binds to CD47 comprises a Fab domain which comprises: a variable light chain (VL) domain amino acid sequence and a variable heavy chain (VH) amino acid sequence as defined herein above in the context of the anti-CD47 antibody as groups of embodiments (i) to (xxvii); a constant light chain (CL) domain comprising an amino acid sequence which is at least about 95%, 96%, 97%, 98%, 99%, or 100% amino acid sequence of SEQ ID NO: 59 and a constant heavy chain (CH1) domain comprising an amino acid sequence which is at least about 95%, 96%, 97%, 98%, 99%, or 100% amino acid sequence of amino acid sequence of SEQ ID NO: 49.

In further preferred embodiments said the anti-CD47 antibody is an IgG4 antibody and comprises a constant light chain domain (CL) and a heavy chain CH1, CH2 and CH3 domain. In further preferred embodiments said anti-CD47 antibody is an IgG4 antibody and comprises a kappa CL domain, preferably a human kappa CL domain and a human heavy chain CH1, CH2, CH3 domain and a hinge domain. The hinge domain is preferably a human hinge region. The present invention further envisages antigen binding fragments of said IgG4 antibody, which specifically bind to CD47.

In further preferred embodiments the IgG4 anti-CD47 antibody or an antigen binding fragment thereof, which specifically binds to CD47, comprises a constant light chain domain (CL) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 59.

In further preferred embodiments the IgG4 anti-CD47 antibody or an antigen binding fragment thereof, which specifically binds to CD47, comprises a constant heavy chain domain 1 (CH1) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 54.

In further preferred embodiments the IgG4 anti-CD47 antibody or an antigen binding fragment thereof, which specifically binds to CD47, comprises a constant heavy chain domain 2 (CH2) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 55.

In further preferred embodiments the IgG4 anti-CD47 antibody or an antigen binding fragment thereof, which specifically binds to CD47, comprises a constant heavy chain domain 3 (CH3) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 56.

In further particularly preferred embodiments, the IgG4 anti-CD47 antibody comprises a constant heavy chain domain 1 (CH1) comprising an amino acid sequence which is at least about 95%, 96%, 97%, 98%, 99%, or 100% identical to the amino acid sequence of SEQ ID NO: 54; and constant heavy chain domain 2 (CH2) comprising an amino acid sequence which is at least about 95%, 96%, 97%, 98%, 99%, or 100% identical to the amino acid sequence of SEQ ID NO: 55; and a constant heavy chain domain 3 (CH3) comprising an amino acid sequence which is at least about 95%, 96%, 97%, 98%, 99%, or 100% identical to the amino acid sequence of SEQ ID NO: 56; and a constant light chain domain (CL) which comprises an amino acid sequence which is at least about 60%, 65%, 70%, 75%, 80%, 85%, 86%, 87%, 88%, 89%, 90%, 91%, 92%, 93%, or 94% identical, preferably about 95%, 96%, 97%, 98%, 99%, or 100% identical to the amino acid sequence of SEQ ID NO: 59.

In further specific embodiments, the IgG4 anti-CD47 antibody comprises CDR sequences as defined herein above, or VH and/or VL sequences as defined herein above together with a CL and a CH1 and a CH2 and a CH3 sequence as defined herein above.

In a further embodiment, the CH2 domain of the IgG4 antibody as defined above and the CH3 domain of the IgG4 antibody as defined above form an Fc domain. Preferably, the Fc domain comprises a CH2 domain comprising an amino acid sequence which is at least about 95%, 96%, 97%, 98%, 99%, or 100% identical to the amino acid sequence of SEQ ID NO: 55 and a CH3 domain comprising an amino acid sequence which is at least about 95%, 96%, 97%, 98%, 99%, or 100% identical to the amino acid sequence of SEQ ID NO: 56. It is further preferred that the Fc domain comprises an amino acid sequence which is at least 9.5%, 96%, 97%, 98% or 99% identical to the amino acid sequence of SEQ ID NO: 97.

In further preferred embodiments the IgG4 anti-CD47 antibody or antigen binding fragment thereof, which specifically binds to CD47 comprises a hinge domain comprising an amino acid sequence which is at least about 95%, 96%, 97%, 98%, 99%, or 100% identical to the amino acid sequence of the amino acid sequence of SEQ ID NO: 57. The term “hinge domain” or alternatively “hinge region” as used herein refers to a functional domain in an antibody, which connects the CH1 and the CH2 domain and thus is between the Fab and Fc domain. It typically comprises two disulphide bonds to dimerize two heavy chains and thereby contributes to the three-dimensional form and structure of an antibody. Its sequence, structure and position provided segmental flexibility to promote the antibody functionality.

In a further embodiment, said CH1, hinge, CH2 and CH3 domain of the IgG4 antibody form the heavy constant chain. It is particularly preferred that said heavy constant chain comprises an amino acid sequence which is at least 95%, 96%, 97%, 98% or 99% identical to the amino acid sequence of SEQ ID NO: 58.

In a further preferred embodiment the IgG4 anti-CD47 antibody or antigen binding fragment thereof, which specifically binds to CD47 comprises a Fab domain which comprises: a variable light chain (VL) domain amino acid sequence and a variable heavy chain (VH) amino acid sequence as defined herein above in the context of the anti-CD47 antibody as groups of embodiments (i) to (xxvii); a constant light chain (CL) domain comprising an amino acid sequence which is at least about 95%, 96%, 97%, 98%, 99%, or 100% amino acid sequence of SEQ ID NO: 59 and a constant heavy chain (CH1) domain comprising an amino acid sequence which is at least about 95%, 96%, 97%, 98%, 99%, or 100% amino acid sequence of amino acid sequence of SEQ ID NO: 54.

In a further set of embodiments, the antigen-binding fragment of the anti-CD47 antibody of the present invention is an anti-CD47 single chain fragment variable (scFv). The term “anti-CD47 single chain fragment variable” or “scFv” refers to a fusion protein of the variable regions of the heavy (VH) and light chains (VL) of immunoglobulins, which are typically connected with a polypeptide linker (scFv linker).

In specific embodiments, the anti-CD47 antibody or antigen binding fragment thereof such as defined above, including IgG1 and IgG4 antibodies, anti-CD47 scFvs and anti-CD47 Fabs as defined herein, which specifically binds to CD47 as defined herein, is specific to a polypeptide comprised by the amino acid sequence of SEQ ID NO: 92 to 95, preferably of SEQ ID NO: 92. In further specific embodiments the anti-CD47 antibody or antigen binding fragment thereof such as defined above, including IgG1 and IgG4 antibodies, anti-CD47 scFv and the anti-CD47 Fab as defined herein which specifically binds to CD47 as defined herein, is capable of binding the extracellular domain of CD47 which refers to amino acids 19-141 of SEQ ID NO: 92. The epitope or binding site on CD47 is similar to or partially overlapping with the binding site for SIRPalpha. To block the interaction between SIRPalpha on an effector cell and CD47 on a tumor cell, the anti-CD47 antibody or antigen binding fragment thereof such as defined above, anti-CD47 scFv and the anti-CD47 Fab as defined herein that specifically binds to CD47 needs to bind the amino acid positions 19-124 of SEQ ID NO: 92 or corresponding parts thereof.

In preferred embodiments of the present invention the anti-CD47 scFv which specifically binds to CD47 comprises a VH region and a VL region comprising CDRs as shown in Table 1 and 2, supra.

In particularly preferred embodiments of the present invention the anti-CD47 scFv which specifically binds to CD47 comprises a VH region comprising the following CDRs:

(1) a CDRH1 sequence selected from the amino acid sequences of SEQ ID NOs: 60 to 62; and (2) a CDRH2 sequence selected from the amino acid sequences of SEQ ID NOs: 63 to 73; and (3) the CDRH3 sequence having the amino acid sequence of SEQ ID NO: 74; or a variant thereof in which one or two or three amino acids in one or more of CDRH1, CDRH2, or CDRH3 are substituted with another amino acid; and comprises a VL region comprising the following CDRs:

(4) a CDRL1 sequence selected from the amino acid sequences of SEQ ID NOs: 75 to 79; and (5) a CDRL2 sequence selected from the amino acid sequences of SEQ ID NOs: 80 and 81; and (6) a CDRL3 sequence selected from the amino acid sequences of SEQ ID NOs: 82 to 85, or a variant thereof in which one or two or three amino acids in one or more of CDRL1, CDRL2, or CDRL3 are substituted with another amino acid.

In further embodiments the anti-CD47 scFv which specifically binds to CD47 preferably comprises a variable light chain amino acid sequence selected from the amino acid sequences of SEQ ID NOs: 1 to 28 and a variable heavy chain selected from the amino acid sequences of SEQ ID NOs: 29 to 48. In further embodiments, the anti-CD47 scFv which specifically binds to CD47 preferably comprises a variable light chain (VL) domain amino acid sequence and a variable heavy chain (VH) amino acid sequence as defined herein above in the context of the anti-CD47 antibody as groups of embodiments (i) to (xxvii).

In preferred embodiments of the present invention the anti-CD47 scFv which specifically binds to CD47 comprises a VH region comprising the amino acid sequence SEQ ID NO: 42 and a VL region comprising the amino acid sequence SEQ ID NO: 15.

Further information on the sequences of the variable light chain region and of the variable heavy chain region of the anti-CD47 IgG1, IgG4 and scFv and the corresponding antibodies can be derived from Table 3, supra. The sequences have been chosen based on affinity to CD47, manufacturability, productivity, developability, immunogenicity, stability and CD47 blocking ability.

In specific embodiments, the VH and VL domains of the anti-CD47 scFv, preferably as shown in Table 3, are connected by an “scFv linker”. The VL and VH domain of the anti-CD47 scFv are provided in the orientation N-terminus-VH-scFv linker-VL-C-terminus or N-terminus-VL-scFv linker-VH-C-terminus, preferably N-terminus-VH-scFv linker-VL-C-terminus. The “scFv linker” may comprise about 10 to about 25 amino acids, preferably 15 to 20 amino acids. The scFv linker may comprise hydrophilic or aliphatic amino acids, preferably threonine, serine, aspartic acid, asparagine, glycine and alanine, which allow for a high flexibility and increased solubility. The “scFv linker” is a flexible linker which reduces inter alia the likelihood that the linker interferes with the folding and function of the individual domains. It is particularly preferred using an “scFv linker” comprising, essentially consisting of or consisting of repeats of the amino acid sequence of SEQ ID NO 86 or SEQ ID NO: 87 or SEQ ID NO: 88 or SEQ ID NO: 89 or SEQ ID NO: 90 or SEQ ID NO: 91, preferably 3-5 repeats of the amino acid sequence of SEQ ID NO: 86 and/or SEQ ID NO: 87. The anti-CD47 scFv according to the present invention retains the specificity of the original or parent anti-CD47 immunoglobulin, despite removal of the constant regions and the introduction of the polypeptide linker.

In further preferred embodiments the anti-CD47 antibodies or antigen-binding fragments thereof such as the anti-CD47 Fabs or the anti-CD47 scFvs according to the present invention have an affinity for CD47 in the range of 100 nM to 2 μM, e. g. an affinity of 100 nM, 200 nM, 300 nM, 400 nM, 500 nM, 600 nM, 700 nM, 800 nM, 900 nM, 1000 nM, 1100 nM, 1200 nM, 1300 nM, 1400 nM, 1500 nM, 1600 nM, 1700 nM, 1800 nM, 1900 nM, or 2000 nM. It is more preferred that the anti-CD47 antibodies or antigen-binding fragments thereof such as the anti-CD47 Fabs or the anti-CD47 scFvs according to the present invention have an affinity for CD47 in the range of 300 nM to 800 nM, e. g. in the range of 400 nM to 700 nm, 500 nM to 800 nM, 400 nM to 600 nm, 400 nM to 700 nM, or 500 mM to 600 nM.

The affinity may be measured with any suitable methodology known to the skilled person including fluorescence titration, enzyme-linked immunosorbent assay (ELISA)-based assays, including direct and competitive ELISA, calorimetric methods, such as isothermal titration calorimetry (ITC), and surface plasmon resonance (SPR). It is preferred that the affinity is measured by SPR. SPR is a technology which is based on resonant oscillation of conduction electrons at the interface between negative and positive permittivity material stimulated by incident light. SPR spectroscopy as envisaged for the definition of affinity values in the context of the present invention allows to monitor the interaction between molecules in real time. The approach typically involves the attaching of one interacting partner to the surface of a sensor chip (ligand), then passing a sample containing the other interaction partner (analyte) over the surface. Binding of molecules to the sensor surface typically generates a response which is proportional to the bound mass. Binding events can accordingly be followed in real time and interaction characteristics can be determined. “Affinity” as used herein is understood as a value of dissociation constant (KD), half maximal effective concentration (EC50), or half maximal inhibitory concentration (IC50) measured using such any of the above-mentioned methods, preferably SPR spectroscopy. Generally, a lower KD, EC50, or IC50 value reflects better (higher) binding ability (affinity).

In particular preferred embodiments, the SPR analysis is performed on Biacore devices as commercialized by GE Healthcare Ltd. or Cytiva.

In specific embodiments the affinity is measured by using standard procedures for SPR spectroscopy, preferably for Biacore devices. Corresponding parameters and technical details would be known to the skilled person or can be derived from suitable literature sources such as Hearty et al., 2012, Methods Mol Biol., 907:411-42. An exemplary and generalizable SPR spectroscopy measurement is also derivable from Example 2, which provides further details on kits and procedures which may be used for SPR-based affinity measurements.

In preferred embodiments, the anti-CD47 antibody or antigen-binding fragment thereof, e. g. the Fab fragment or the anti-CD47 scFv as defined herein, has an affinity for its target CD47, which in comparison to the affinity of a reference monoclonal antibody to its target CD47, is lower by a factor of at least 20, e. g. 20, 25, 27, 30, 32, 35 or more, more preferably by a factor of at least 40, e. g. 45, 50, even more preferably by a factor of at least 60, e.g. 60, 70, 80, 90, 100 or more. An example of a suitable and envisaged reference antibody is B6H12. B6H12 is a mouse monoclonal IgG1 K (kappa) anti-CD47 antibody. It can be obtained from ATCC under ATCC deposition number HB 9771 or commercial suppliers such as Santa Cruz Biotechnology. Further information on the antibody can be derived, for example, from U.S. Pat. No. 5,057,604 or Subramanian et al, Blood 2006, Mar. 15; 107(6): 2548-2556. The affinity measurement in order to determine the affinity of an antibody of the invention in comparison to a reference antibody such as B6H12 is preferably performed with SPR methods as defined above, more preferably with a Biacore device using standard parameter, e. g. as disclosed in Example 2.

Particularly, the anti-CD47 antibody or antigen-binding fragment thereof, e. g. the Fab fragment or anti-CD47 scFv as defined herein may be captured to an SPR sensor chip, preferably a CM5 chips using e.g. a human Fc capturing kit, and the extracellular domain of CD47 is used as analyte.

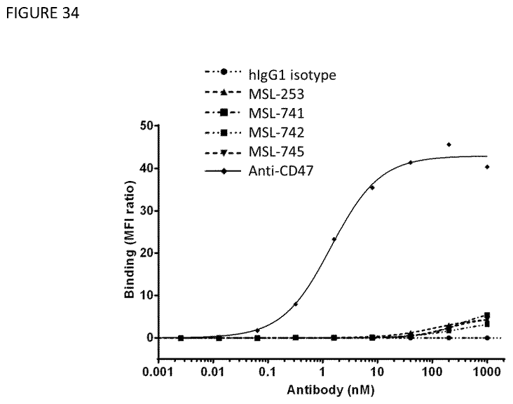

In a further preferred embodiment said anti-CD47 antibody or antigen-binding fragment thereof, e. g. the Fab fragment or anti-CD47 scFv as defined herein does not bind to red blood cells (RBCs) at concentrations below 50 nM as measured by flow cytometry. The term “no binding” as mentioned herein means that the ratio of the mean fluorescence intensity (MFI) of said anti-CD47 antibody or antigen-binding fragment thereof to the MFI of the isotype control antibody is below 1.5. Further information can be derived from the Examples and Figures, in particular Example 8 and FIG. 34, or from suitable literature sources.

In a further preferred embodiment said anti-CD47 antibody or antigen-binding fragment thereof, e. g. the Fab fragment or anti-CD47 scFv as defined herein does not induce platelet aggregation. The term “not induce platelet aggregation” as mentioned herein means that the anti-CD47 antibody or antigen-binding fragment thereof does not crosslink platelets. This “cross-link” might occur by binding to CD47 and Fc receptors (FcRs), especially Fc gamma RIIA (CD32a) expressed on PLTs. Further information may be derived from suitable literature sources such as Arman et al., 2015, J Thromb Haemost, 13(6), 893-908. This property is of elevated importance since platelet aggregation can lead to thrombocytopenia or similar diseases and has be observed with some CD47 targeting agents. It is preferred that said anti-CD47 antibody or antigen-binding fragment thereof does not exceed 20% of platelet aggregation, using platelet rich plasma (PRP) and platelet poor plasma (PPP) and where the percentage of platelet aggregation is measured every 15 s by the absorbance at a wavelength of 595 nm between various shakings over 30 minutes (min) at 37° C. Changes in absorbance are converted to percentage of aggregation by reference to the absorbances of PRP and PPP by the formula:

$$\% platelet aggregation = [ ( OD PRP - OD sample ) / ( OD PRP - OD PPP ) ] \times 100.$$

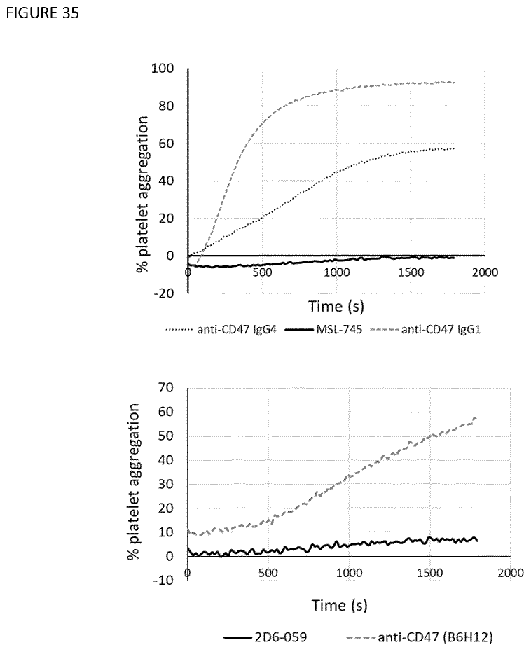

For example, a number of about 20%, 15%, 10%, 8%, 6%, 4%, 2%, or 1% or any value in between the mentioned values, of the PLTs in a certain volume, e.g. 100 μl is aggregated by the anti-CD47 antibody or antigen-binding fragment as defined herein. It is further preferred that the property of PLT aggregation is measured at a concentration of about 10 mM to 1000 nM of said anti-CD47 antibody or antigen-binding fragment thereof. It is particularly preferred that the property of PLT aggregation is measured at a concentration of about 100 mM of said anti-CD47 antibody or antigen-binding fragment thereof. Further information can be derived from the Examples and Figures, in particular Example 9 and FIG. 35, or from suitable literature sources.

In a further even more preferred embodiment said anti-CD47 antibody or antigen-binding fragment thereof as defined herein leads to less than 20% platelet aggregation using PRP and PPP measured by the absorbance at a wavelength of 595 nm over 30 minutes at 37° C. and calculated by the formula:

$$\% platelet aggregation = [ ( OD PRP - OD sample ) / ( OD PRP - OD PPP ) ] \times 100 ,$$

preferably in a concentration range of 10 nM to 1000 nM, more preferably at 100 nM.

In a further aspect the present invention relates to an anti-CD47 antibody or antigen-binding fragment as defined herein above, wherein the anti-CD47 antibody or antigen-binding fragment is combined to a further functional component. The term “functional component” as used herein refers to a polypeptide entity which provides a biological function, preferably is capable of binding to a target, e. g. a surface protein on a cell. The term “combined” as used herein comprises in one group of embodiments the physical combination of the anti-CD47 antibody or antigen-binding fragment thereof in a single molecule, e.g. in the form of a polypeptide fusion. The term also relates, in a different group of embodiments, to two distinct molecules, i.e. physically separated molecules, being present in one pharmaceutical composition or being administered in two or more individual pharmaceutical compositions, e.g. as a kit of parts, at the same time or in the same treatment cycle according to the administration scheme.

It is particularly preferred that the functional component is a binding domain for a tumor marker present on the surface of a tumor cell. The term “binding domain” as used herein relates to a polypeptide or fragment thereof which is independently capable of binding to a target, e. g. a further protein. Generally, a binding domain is a part of a polypeptide with a tertiary structure which can change and function by itself independent of the rest of the polypeptide chain. Upon binding, binding domains may undergo a conformational change. It is further preferred that the binding domain specifically binds to a target. In certain embodiments, the binding domain comprises immunoglobulin elements, e. g. CDRs and FRs of an antibody, which allow for a specific binding to a target. In further preferred embodiments, the binding domain may comprise a Fab fragment of an antibody or constitute an scFv comprising a variable light chain (VL) and a variable heavy chain (VH) as defined herein. It is further envisaged that the anti-CD47 antigen-binding fragments of the present invention described above, such as the anti-CD47 Fabs or the anti-CD47 scFvs which specifically bind to CD47 are combined to an IgG specific for a tumor marker, preferably a tumor antigen different than CD47, such as mesothelin (MSLN), CEACAM5, B7H3, CA125, EGFR, Her2 or mucin-1. In such embodiments the further functional component may be, for example, a tumor specific IgG antibody such as an IgG1 or IgG4. In further embodiments, an anti-CD47 antibody, e. g. an IgG1 or IgG4 antibody as defined herein, may be combined with a Fab fragment of an antibody, or an scFv comprising a variable light chain (VL) and a variable heavy chain (VH) which are specifically binding to a tumor marker, preferably a tumor antigen different than CD47, such as mesothelin (MSLN), CEACAM5, B7H3, CA125, EGFR, Her2 or mucin-1.

The term “tumor marker” or “tumor antigen” as used herein refers to a protein or polypeptide, which is elevated in the presence of one or more types of cancer. The tumor marker may be indicative for a particular cancer developmental stage or cancer type. It is preferred that the tumor markers as used in the context of the present invention are expressed by cancer cells. It is further preferred that the tumor markers as used in the context of the present invention are presented on the surface of a cell, e. g. as transmembrane protein or receptor or receptor ligand. Examples of suitable tumor markers include, B7H3, bladder tumor-associated antigen, CA 15-3, CA 19-9, CA 27-29, CA125, carcinoembryonic antigen (CEACAM5), c-Kit, EMA, EpCAM, cytokeratin 8, cytokeratin 18, cytokeratin 19, CD2, CD19, CD22, CD29, CD30, CD33, CD37, CD38, CD40, CD56 (N-CAM), CD70, CD74, CD138, CD124, CD319 (SLAMF7), desmin, epidermal growth factor receptor (EGFR), estrogen receptor (ER), HMB-45, HER2/NEU, HER3, FAP, GD2, GD3, FOLR1, gpA33, IGF1R, FGF3, MET (HGFR), mesothelin (MSLN), MUC-4, CA125 (MUC-16), TROP-2, SLC34A2, Folate Receptor, mucin-1, progesterone receptor (PR), PSA.

It is particularly preferred that the tumor marker is one of: mesothelin (MSLN), CEACAM5, B7H3, CA125, EGFR, Her2 or mucin-1.

In a further preferred embodiment said combination of an anti-CD47 antibody or antigen-binding fragment thereof as defined herein above to a further functional component, which is capable of binding to a target, such as a tumor marker, preferably by a binding domain, is a connection of the two components by a polypeptide linker.

The polypeptide linker as envisaged by the present invention thus connects the anti-CD47 antibody or antigen-binding fragment thereof, e. g. the Fab fragment or scFv as defined herein, to said functional component, preferably a tumor marker binding domain or more preferably to a tumor marker binding antibody, in the form of a protein fusion, i. e. the functional component is fused via the polypeptide linker to an anti-CD47 antigen-binding fragment. Similarly, a functional component according to the present invention, e. g. a polypeptide which is capable of binding to a tumor marker such as a tumor marker binding IgG antibody or tumor marker binding fragment thereof may be connected by the polypeptide linker to an anti-CD47 antibody, as defined herein in the form of a protein fusion. The exact connection sites at both ends of the domain linker may vary according to the linker length, the form of the component or other factors.

In case of a fusion to a full anti-CD47 IgG1 it is preferred that the polypeptide linker is fused to either the N-terminus of the variable light (VL) chain or the N-terminus of the variable heavy (VH) chain or to the C-terminus of the constant light (CL) or the C-terminus of the constant heavy (CH3) chain domain of the anti-CD47 antibody. In case of an anti-CD47 antigen-binding fragment, e. g. an scFv as defined herein, the CD47 antigen-binding fragment, e. g. scFv, is fused either to the N-terminus of the variable light (VL) chain or to the N-terminus of the variable heavy (VH) chain or to the C-terminus of the constant light (CL) chain or to the C-terminus of the constant heavy (CH3) chain domain of the target binding IgG antibody. It is particularly preferred that the scFv is fused to the N-terminus of the variable light (VL) chain. Besides the function of connecting or fusing the anti-CD47 antibody or antigen binding fragment thereof to a different domain, the polypeptide linker further allows to spatially separate, for instance, the tumor marker binding and the CD47-binding domains. This advantageously allows for a simultaneous binding of a tumor marker target and CD47, which may be present at the cell surface. The density and numbers of a tumor marker target and CD47 on the cell surface varies between different cell lines, tumor cells and cell types.

In another preferred embodiment said binding to CD47 is enhanced and/or reinforced by the protein construct's concomitant, non-exclusive binding of the IgG1 antibody or fragment thereof to a tumor target marker present on the cell surface of a tumor cell, for example by the binding of MSLN on MSLN and CD47 (MSLN⁺/CD47⁺) double positive tumor cells.

In a particularly preferred embodiment said enhancement and/or reinforcement is caused by an increased avidity of the protein construct caused by the multivalent binding to different antigens, such as MSLN and CD47, on one cell.

The term “avidity” as used herein is known to the skilled person and generally relates to the accumulated strength of multiple monovalent affinities of individual noncovalent binding interactions. Individually, each binding interaction contributes with its affinity to an overall binding strength known as avidity. As such, avidity is distinct from affinity, which describes the strength of monovalent (single epitope) binding.

In further embodiments the polypeptide linker is flexible. The term “flexible” as used herein means that the linker polypeptide has a high degree of conformational freedom, which is assumed to prevent the formation of unwanted secondary structures, to reduce the likelihood that the linker interferes with the folding and function of the binding domains and to allow for a spatially variable interaction with two targets, and simultaneous binding of a tumor marker target and CD47 at the cell surface, i.e. to bind to a tumor marker target and CD47 being at different distances to each other.

The polypeptide linker may have any suitable length and flexibility allowing for a simultaneous binding to the tumor marker target and CD47 and/or allowing for prevention of the formation of unwanted secondary structures, and/or allowing for reduction of the likelihood that the linker interferes with the folding and function of the binding domains and/or allowing for a spatially variable interaction with the tumor marker target and CD47. A “simultaneous binding” as used herein refers to the state after an interaction of the construct according to the invention with the tumor marker target and the CD47 target has taken place, i. e. it means that the construct according to the present invention is connected to both targets, a tumor marker target and CD47. It may be based on different dynamics in the binding process, e. g. a temporally first binding of the construct to the tumor target, followed by a binding to CD47, or vice versa, or a temporally synchronous binding of the tumor marker and CD47.

In certain embodiments of the present invention the polypeptide linker comprises 4 to 40 amino acids, e. g. 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids. In very specific embodiments, the polypeptide linker may also be longer. In additional embodiments, the polypeptide linker essentially consists of, or consists of 4 to 40 amino acids, e. g. consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acids.

The polypeptide linker may be composed of any suitable amino acid which fulfils at least one or, preferably, more or all of the above mentioned functions, i.e. fusing the components and spatially separating the components, allowing simultaneous binding of the construct to the tumor marker and CD47, prevention of the formation of unwanted secondary structures, reduction of the likelihood that the polypeptide linker interferes with the folding and function the domains and allowing for a spatially variable interaction with the targets on the cell surface. According to preferred embodiments, the polypeptide linker comprises, essentially consist of, or consists of the amino acid glycine, alanine, proline, lysine and/or serine. It is particularly preferred that the polypeptide linker comprises, essentially consist of, or consists of the amino acid glycine and/or serine. For example, the polypeptide linker may comprise 0%, 10%, 20%, 30%, 40%, 50%, 60%, 70%, 80%, 90% or 100% glycine, or 0%, 10%, 20%, 30%, 40%, 50%, 60%, 70%, 80%, 90% or 100% serine. The amino acids may be located at any position in the polypeptide linker and the succession of amino acids may be suitable succession, e. g. starting with glycine, followed by serine etc., or vice versa. Further, the polypeptide linker may comprise stretches of identical amino acids, e. g. stretches of about 5, 10, 15, 20, 25, 30, 35 or more glycines or serines. Further details would be known to the skilled person or can be derived from suitable literature sources such as van Rosmalen et al., 2017, Biochemistry, 56, 6565-6574 or Chen et al, 2013, Adv Drug Deliv Rev, 65(10): 1357-1369.

In further particularly preferred embodiments, the polypeptide linker may comprise, essentially consist of or consist of one or more of the amino acid sequences of SEQ ID NOs: 86 to 91 as shown in the following Table 4, or any combination or multiplication thereof:

TABLE 4

| Name | Sequence | SEQ ID NO |
|---|---|---|
| Linker 1 | GGGGS | 86 |
| Linker 2 | GGGS | 87 |
| Linker 3 | GGGGG | 88 |
| Linker 4 | GGGG | 89 |
| Linker 5 | GGS | 90 |
| Linker 6 | GS | 91 |

For example, the amino acid sequence of Linker 1 (SEQ ID NO: 86) may be present in the polypeptide Linker one time, 2 times, 3 times, 4 times, 5 times, 6 times, 7 times or 8 times. One or more copies of the amino acid sequence of Linker 1 (SEQ ID NO: 86) may further be combined with one or more copies of Linker 2 (SEQ ID NO: 87), Linker 3 (SEQ ID NO: 88), Linker 4 (SEQ ID NO: 89), Linker 5 (SEQ ID NO: 90) or Linker 6 (SEQ ID NO: 91) in a random order.

In a further embodiment the amino acid sequence of Linker 2 (SEQ ID NO: 87) may be present in the polypeptide Linker one time, 2 times, 3 times, 4 times, 5 times, 6 times, 7 times, 8 times, 9 times or 10 times. One or more copies of the amino acid sequence of Linker 2 (SEQ ID NO: 87) may further be combined with one or more copies of Linker 1 (SEQ ID NOs: 86), Linker 3 (SEQ ID NO: 88), Linker 4 (SEQ ID NO: 89), Linker 5 (SEQ ID NO: 90) or Linker 6 (SEQ ID NO: 91).

In a further embodiment the amino acid sequence of Linker 3 (SEQ ID NO: 88) may be present in the polypeptide Linker one time, 2 times, 3 times, 4 times, 5 times, 6 times or 7 times. One or more copies of the amino acid sequence of Linker 3 (SEQ ID NO: 88) may further be combined with one or more copies of Linker 1 (SEQ ID NOs: 86), Linker 2 (SEQ ID NO: 87), Linker 4 (SEQ ID NO: 89), Linker 5 (SEQ ID NO: 90) or Linker 6 (SEQ ID NO: 91).

In a further embodiment the amino acid sequence of Linker 4 (SEQ ID NO: 89) may be present in the polypeptide Linker one time, 2 times, 3 times, 4 times, 5 times, 6 times, 7 times, 8 times, 9 times or 10 times. One or more copies of the amino acid sequence of Linker 4 (SEQ ID NO: 89) may further be combined with one or more copies of Linker 1 (SEQ ID NOs: 86), Linker 2 (SEQ ID NO: 87), Linker 3 (SEQ ID NO: 88), Linker 5 (SEQ ID NO: 90) or Linker 6 (SEQ ID NO: 91).

In a further embodiment the amino acid sequence of Linker 5 (SEQ ID NO: 90) may be present in the polypeptide Linker two times, 3 times, 4 times, 5 times, 6 times, 7 times, 8 times, 9 times, 10 times, 11 times, 12 times or 13 times. One or more copies of the amino acid sequence of Linker 5 (SEQ ID NO: 90) may further be combined with one or more copies of Linker 1 (SEQ ID NOs: 86), Linker 2 (SEQ ID NO: 87), Linker 3 (SEQ ID NO: 88), Linker 4 (SEQ ID NO: 89) or Linker 6 (SEQ ID NO: 91).

In a further embodiment the amino acid sequence of Linker 6 (SEQ ID NO: 91) may be present in the polypeptide linker two times, 3 times, 4 times, 5 times, 6 times, 7 times, 8 times, 9 times, 10 times, 11 times, 12 times, 13 times, 14 times, 15 times, 16 times, 17 times, 18 times, 19 times or 20 times. One or more copies of the amino acid sequence of Linker 6 (SEQ ID NO: 91) may further be combined with one or more copies of Linker 1 (SEQ ID NOs: 86), Linker 2 (SEQ ID NO: 87), Linker 3 (SEQ ID NO: 88), Linker 4 (SEQ ID NO: 89) or Linker 5 (SEQ ID NO: 90).

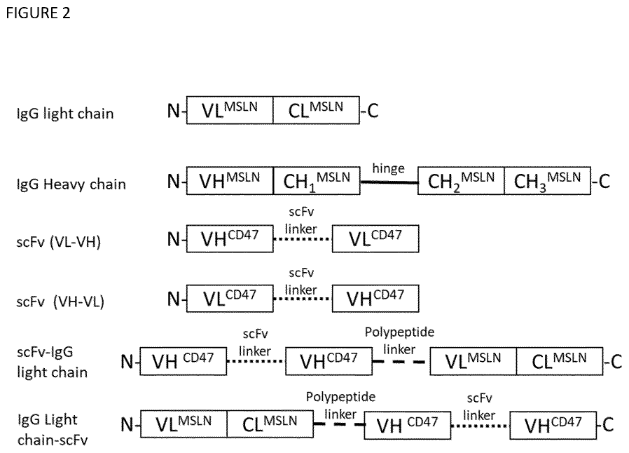

FIG. 2 provides examples of the structural orientation of domains as envisaged by the present invention.

In a further aspect the present invention relates to a nucleic acid molecule comprising a polynucleotide encoding the anti-CD47 antibody or antigen-binding fragment as defined herein, including constructs comprising the anti-CD47 antibody or antigen-binding fragment and a further functional domain, preferably binding to a tumor marker as defined herein. Also envisaged is a nucleic acid molecule comprising a polynucleotide encoding a fragment of the anti-CD47 antibody, preferably a functional fragment of the anti-CD47 antibody as defined herein or of a construct comprising the anti-CD47 antibody or antigen-binding fragment and a further functional domain. Further envisaged are nucleic acid molecules encoding fragments or components of the anti-CD47 antibody e. g. a heavy chain (HC) and a light chain (LC) wherein the encoded fragments or components are not fused via peptide bonds, or via a polypeptide Linker, e. g. constitute different chains of an antibody together forming an antibody or Fab fragment or Fc domain.

The term “nucleic acid” or “nucleic acid molecule” as used herein refers to any nucleic acid known to the person skilled in the art, e. g. a polynucleotide like DNA, RNA, single stranded DNA, cDNA, or derivatives thereof. The nucleic acid can further be linear or circular. Preferably, the term refers to DNA polynucleotides.

The nucleic acid molecule comprising a polynucleotide encoding the anti-CD47 antibody or antigen-binding fragment as defined herein, including constructs comprising the anti-CD47 antibody or antigen-binding fragment and a further functional domain may provide any sequence variant which encodes said antibody, fragment or combination construct as defined herein, e. g. making use of one or more different codons for an amino acid. It is particularly preferred that the nucleic acid comprises a sequence which has been optimized to an organism in which the sequence is planned to be expressed. This “codon-optimization” may be adapted to host organisms according to information on the codon usage in the corresponding organism. Further, codons or codon combinations having an influence on the transcription and/or translation processes, e. g. constituting binding motifs etc., may preferably be avoided in the optimization process.

The nucleic acid molecule comprising a polynucleotide encoding the anti-CD47 antibody or antigen-binding fragment as defined herein, including constructs comprising the anti-CD47 antibody or antigen-binding fragment and a further functional domain may be obtained by any suitable method. For example, suitable antibodies or fragments thereof as described herein, may be isolated and sequenced, e. g. by using conventional procedures, to obtain the encoding nucleic acid sequence, or, preferably, the nucleic acid molecule may be synthesized synthetically, for instance by using conventional procedures, on the basis of existent nucleic acid sequence information. Furthermore, the nucleic acid molecule may be modified and changed in accordance with antibody modification procedures as described herein, e. g. its sequence may be changed by modifying domain sequences, swapping domain sequences, combining sequences encoding CDRs and FRs, humanizing the sequences, inserting point mutations etc. Typically, recombinant DNA techniques and procedures as known to the skilled person may be used to generate, modify or optimize the nucleic acid molecule for envisaged purposes such as expression in certain cells or organism etc. Suitable references include Green and Sambrook, Molecular Cloning: A Laboratory Manual (4th Edition), Cold Spring Harbor Press, 2012.

In a further aspect the present invention relates to a vector comprising the nucleic acid molecule as described above. The term “vector” as used herein refers a nucleic acid molecule that can be used as a vehicle to transfer (heterologous) genetic material into a cell. Such a vehicle may be, for example, a plasmid, a virus, a cosmid, an artificial chromosome, an episome or the like. The vector itself is generally a molecule comprising a nucleotide sequence, typically a DNA sequence that comprises an insert (e. g. a transgene) and a larger sequence that serves as the backbone of the vector. Vectors may encompass additional features besides the transgene insert and a backbone such as one or more promoters, one or more genetic markers, an antibiotic resistance, a reporter gene, a targeting sequence, a protein purification tag.

In preferred embodiments the vector is an expression vectors, i. e. a vehicle comprising a nucleic acid as defined above, which is specifically designed for the expression of the transgene in a target or host cell. An expression vector generally comprises a control sequence such as a promoter sequence that drives expression of the transgene. The term “control sequence” as used herein refers to a DNA sequence necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers. A nucleic acid is “operably linked” when it is placed into a functional relationship with another nucleic acid sequence. For example, DNA for a pre-sequence or secretory leader is operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. The term “expression” as used herein relates to any step known to the skilled person which is involved in the production of an anti-CD47 antibody or antigen-binding fragment as defined herein, including constructs comprising the anti-CD47 antibody or antigen-binding fragment and a further functional domain according to the invention including, transcription, post-transcriptional modification, translation, posttranslational modification, and secretion. It is further preferred that the vector encodes the anti-CD47 antibody or antigen-binding fragment as defined herein, including constructs comprising the anti-CD47 antibody or antigen-binding fragment and a further functional domain according to the invention in any suitable way, e. g. by comprising restriction sites so that domains or sequence fragments can be introduced or removed. Examples of vectors to be used in the context of the present invention include to be used in the context of the present invention include pFUSE-CHIg-hG1, pFUSE-CLIg-hk, pFUSE-CHIg-hG4, pSecTag, pQE70, pQE60, pQE9, pcDNA3.1, pNH8A, pNH16a, pNH18A, pNH46A, pCI-Neo, pCMV, pcDNA3.4, pKK223-3, pKK233-3, pDR540, pRIT5, pET, pGEX2TK, pGEX-4T, pGEX-5X-1, pMAL, pWLNEO, pSV2CAT, pOG44, and pSG, pGS, pETDuet, pCDFDuet-1, or pRSFDuet-1.

In a further aspect the present invention relates to a host cell comprising the nucleic acid molecule or the vector of the present invention. The term “host cell” or “target cell” is intended to refer to any individual cell or cell culture that can be or has/have been recipients for vectors or the incorporation of exogenous nucleic acid molecules, polynucleotides and/or proteins. It also is intended to include progeny of a single cell. The cell may be prokaryotic or eukaryotic, and include bacteria, yeast cells, fungi, insect cells, animal cells, and mammalian cells, e. g., murine, rat, sheep, goat, or human. For example, the protein construct of the invention can be produced in prokaryotes such as bacteria or eukaryotes such as Chinese Hamster Ovarian (CHO) cells or specialized and adapted clones thereof. After expression, the protein construct may be isolated from the host cell and can subsequently be purified through, e. g., affinity chromatography, ion-exchange chromatography and/or size exclusion chromatography. Final purification can be carried out similar to the process for purifying antibody expressed e. g, in CHO cells. Particularly preferred host cells are those which allow for the expression of glycosylated protein constructs. Such host cells are typically derived from multicellular organisms. Examples of invertebrate cells include plant and insect cells. For example, the host cell may be a baculoviral strain infected permissive insect cell such as Spodoptera frugiperda, Aedes aegypti, Aedes albopictus, Drosophila melanogaster, and Bombyx mori. Particularly preferred are vertebrate cells, including mammalian host cell lines. Envisaged examples are monkey kidney CV1 cell line, human embryonic kidney line, baby hamster kidney cells (BHK); Chinese hamster ovary cells (CHO), mouse Sertoli cells, VERO-76 cells, HELA cells, canine kidney cells (MDCK), or human lung cells (W138). Particularly preferred are CHO cells or cells with CHO background, e. g. ExpiCHO, CHO-DG44, CHO-K1, or CHO-S. Further preferred are HEK cells or cells with HEK background, e. g. HEK293, HEK293T, Expi293. Also preferred are HighFive cells, Sf9 cells and Sf21 cells.

In a further aspect the present invention also relates to a host cell expressing anti-CD47 antibody or antigen-binding fragment as defined herein, including constructs comprising the anti-CD47 antibody or antigen-binding fragment and a further functional domain as defined above. Accordingly, the antibody or construct of the invention can be produced intracellularly, in the periplasmic space, or directly secreted into the medium. If the antibody or construct is produced intracellularly, as a first step, the particulate debris, either host cells or lysed fragments, are removed, for example, by centrifugation or ultrafiltration. Where the antibody or construct is secreted into the medium, supernatants from the expression systems is purified directly from the supernatants from the expression systems by affinity chromatography. Subsequently, the antibody or construct of the invention prepared from the host cells can be purified using, for example, exchange or size exclusion chromatography, dialysis, and affinity chromatography, with affinity chromatography being the preferred purification technique.

In a further aspect the invention envisages a method of producing the antibody or construct protein comprising the cultivation of a host cell, thereby expressing the protein. The term “cultivation” refers to the in vitro maintenance, differentiation, growth, proliferation and/or propagation of cells under suitable conditions in a medium. The medium may, for example, comprise suitable carbon sources such as glucose, dextrose, mannitol, fructose, or mannose, which are provided in a suitable concentration, e. g. between 10 g/L to 150 g/L. The medium may further comprise antibiotics such as but not limited to G418 sulfate, Zeocin, hygromycin B, puromycin, blasticidin, and neomycin in any suitable concentration. The medium may further have a specific pH and comprise certain amounts of trace elements. Cultivation conditions may further be adapted to the size and form of fermentation or growth. For example, the cultivation may be a batch fermentation process, or a continuous or perfusion growth approach which envisages the continuous addition of fresh media. Further details would be known to the skilled person or can be derived from suitable literature sources such as Rodrigues et al., 2010, Biotechnol Prog, 26(2), 332-51.

The present invention further relates in an additional aspect to the product produced by the method as described above. The product may, for example, have a specific form or conformation which is due to the host cell used or activities within said host cell. For example, the product may be specifically glycosylated or be not glycosylated, e. g. if expressed in a mammalian or a prokaryotic host cell, respectively or otherwise contain post-translational modifications. The product may further be provided in different degrees of purity, e. g. the product may contain host cell protein or DNA, product degradation products or product aggregates depending on the purification method used.

In another aspect the present invention encompasses a pharmaceutical composition comprising the anti-CD47 antibody or antigen-binding fragment as defined herein, including constructs comprising the anti-CD47 antibody or antigen-binding fragment combined to a further functional domain, or the product as defined above together with a pharmaceutically acceptable carrier or optionally a pharmaceutical adjuvant. The term “pharmaceutical composition” as used herein relates to a composition for administration to a patient, preferably a human patient. The preferred pharmaceutical composition of this invention comprises the anti-CD47 antibody or antigen-binding fragment as defined herein, including constructs comprising the anti-CD47 antibody or antigen-binding fragment and a further functional domain of the invention.

In a further specific embodiment, the pharmaceutical composition comprises an anti-CD47 antibody or an antigen-binding fragment thereof such as an anti-CD47 Fab or an anti-CD47 scFv, which are not combined or fused to further functional components, and wherein the pharmaceutical composition additionally comprises either:
(i) an antibody or antigen-binding fragment thereof targeting Mesothelin (MSLN); or
(ii) an antibody or antigen-binding fragment thereof targeting CEACAM5; or
(iii) an antibody or antigen-binding fragment thereof targeting B7H3; or
(iv) an antibody or antigen-binding fragment thereof targeting CA125; or
(v) an antibody or antigen-binding fragment thereof targeting Mucin-1.
(vi) an antibody or antigen-binding fragment thereof targeting EGFR; or
(vii) an antibody or antigen-binding fragment thereof targeting Her2.

The term “mesothelin” or “MSLN” as used herein relates to a mesothelin polypeptide and fragments thereof which may be present on the surface of cells. Mesothelin has been described as glycophosphatidylinositol (GPI)-linked cell-surface glycoprotein, which is typically synthesized as a 71 kDa precursor protein and is then cleaved to release a secreted N-terminal region, called megakaryocyte potentiating factor (MPF). (Yamaguchi et al, 1994, J Biol Chem.; 269(2):805). The 41 kDa mature MSLN remains at the cell surface attached to the cell membrane (Chang et al, 1996, Proc Natl Acad Sci USA.; 93(1):136).

Human mesothelin is identified by UniProt Q13421 and is also known as CAK1 antigen or Pre-pro-megakaryocyte-potentiating factor. Alternative splicing of mRNA encoded by the human MSLN gene yields four isoforms which differ in length: isoform 1 (UniProt: Q13421-1, SEQ ID NO: 98) which has been chosen as canonical isoform in UniProt and which is used as reference isoform for the numbering of the protein (and the numbering of the other isoforms), but reflects a minor form; isoform 2 (Q13421-3, SEQ ID NO: 99), which lacks amino acids corresponding to positions 409 to 416 of SEQ ID NO: 98 and is the major form; isoform 3 (UniProt: Q13421-2, SEQ ID NO: 100), which lacks amino acids corresponding to positions 409 to 416 of SEQ ID NO: 98 and comprises sequence changes in the stretch of amino acids corresponding to positions 601 to 630 of SEQ ID NO: 98; and isoform 4 (UniProt: Q13421-4, SEQ ID NO: 101) lacks amino acids corresponding to positions 44 and 409 to 416 of SEQ ID NO: 98. Amino acids corresponding to positions 1 to 36 of SEQ ID NO: 98 have been identified as signal peptide, amino acids corresponding to positions 607 to 630 of SEQ ID NO: 98 have been identified as pro-peptide which is removed in a mature form, amino acids corresponding to positions 37 to 606 of SEQ ID NO: 98 are typically considered to constitute the canonical mesothelin, amino acids corresponding to positions 37 to 286 of SEQ ID NO: 98 are typically considered to constitute the megakaryocyte-potentiating factor fragment of mesothelin and amino acids corresponding to positions 296 to 606 of SEQ ID NO: 98 constitute the processed form of mesothelin, which is presented at the surface of a cell. Both MPF and the processed form of mesothelin are typically N-glycosylated. The glycosylation sites are at positions 57 (in MPF), 388, 496 and 523 (in mesothelin) of SEQ ID NO 98.

The structure and function of mesothelin is described, for example, in Hassan et al., 2004, Clin Cancer Research 10, 3737.

The term “B7H3” as used relates to a B7H3 polypeptide and fragments thereof which may be present on the surface of cells. B7H3 has been described as a type I membrane protein of 57 kDa. B7H3 was found to be overexpressed in several types of human cancers and was correlated with disease progression. B7H3 is recognized as a co-stimulatory molecule for immune reactions such as T cell activation and IFN-γ production.

Human B7H3 is identified by UniProt Q5ZPR3 and is also known as CD276, 4IgB7-H3, B7RP-2 and B7 homolog 3. There are four isoforms of different length, whereby isoform 1 is the longest one (UniProt: Q5ZPR3-1, SEQ ID NO: 104) comprising 534 amino acids, corresponding to the major form and which has been chosen as canonical isoform in UniProt and which is used as reference isoform for the numbering of the protein (and the numbering of the other isoforms). Isoform 2 (UniProt: Q5ZPR3-2, SEQ ID NO: 105) lacks amino acids corresponding to positions 159-376 of SEQ ID NO: 104. Isoform 3 (UniProt: Q5ZPR3-3, SEQ ID NO: 106) lacks amino acids corresponding to positions 494-534 of SEQ ID NO: 104 and comprises sequence changes in the stretch of amino acids corresponding to positions 465-493 of SEQ ID NO: 104. Isoform 4 (UniProt: Q5ZPR3-4, SEQ ID NO: 107) comprises sequence changes in the stretch of amino acids corresponding to positions 528-534 of SEQ ID NO: 104. Amino acids 29-466 of SEQ ID NO: 104 comprise the extracellular domain of B7H3, which comprises the epitope of said antibody binding to B7H3.

The structure and function of B7H3 is described, for example, in Vigdorovich et al., 2013, Structure, 21(5): 707.

The term “CEACAM5” as used herein relates to a CEACAM5 polypeptide and fragments thereof which may be present on the surface of cells. CEACAM5 has been described as a GPI-linked cell surface glycoprotein which is glycosylated at more than 30 different sites that represents the founding member of the carcinoembryonic antigen (CEA) family of proteins. CEACAM5 is used as a clinical biomarker for gastrointestinal cancers and may promote tumor development through its role as a cell adhesion molecule. Additionally, CEACAM5 can regulate differentiation, apoptosis, and cell polarity. Human CEACAM5 is identified by UniProt P06731 and is also known as Carcinoembryonic antigen-related cell adhesion molecule 5, Carcinoembryonic antigen, CEA, Meconium antigen 100 and CD66e. There are two isoforms of different length, whereby isoform 1 is the longest one (UniProt: P06731-1, SEQ ID NO: 108) comprising 702 amino acids, corresponding to the major form and which has been chosen as canonical isoform in UniProt and which is used as reference isoform for the numbering of the protein (and the numbering of the other isoforms). Isoform 2 (UniProt: P06731-2, SEQ ID NO: 109) lacks the amino acid corresponding to positions 320 of SEQ ID NO: 108. Amino acids 35-675 of SEQ ID NO: 108 comprise the extracellular domain of CEACAM5, which comprises the epitope of said antibody binding to CEACAM5. The pro-peptide of amino acid 686-702 is removed in the mature form.

The structure and function of CEACAM5 is described, for example, in Hammarström et al., 1999, Seminars in Cancer Biology, 9(2), 67.

The term “CA125” as used relates to a CA125 polypeptide and fragments thereof which may be present on the surface of cells. CA125 has been described as an unusually large single transmembrane protein. As all mucins, it includes tandem repeats of the PTS domain (domain with a high proportion of prolines, threonines and serines) which is extensively glycosylated through GalNAc O-linkages at the threonine and serine residues and has some homology to the SEA (Sea-urchin sperm protein, Enterokinase and Agrin) module. CA125 contains more than 60 such repeats. The epitopes for known anti-CA125 antibodies (OC125 and M11) are thought to be present on a small cysteine rich region present in the tandem-repeat region of CA125. CA125 is overexpressed in around 80% of epithelial ovarian cancers, is detectable in the serum of patients with ovarian cancer and used as a well-established marker to diagnose ovarian cancer. CA125 binds to mesothelin and has been postulated to confer adhesion of ovarian cancer cells to the peritoneum. Human CA125 comprises 14507 amino acids and is identified by UniProt Q8WXI7 and SEQ ID NO: 110 and is also known as Ovarian cancer-related tumor marker CA125, CA125, Mucin-16 and MUC-16. Amino acids 1-14451 of SEQ ID NO: 110 comprise the extracellular domain of CA125, which comprises the epitope of said antibody binding to CA125.

The structure and function of the Mucin family including CA125 and other members is described, for example, in Kufe et al., 2009, Nat Rev Cancer; 9(12): 874.

The term “Mucin-1” as used herein relates to a Mucin-1 polypeptide and fragments thereof which may be present on the surface of cells. Human Mucin-1 comprises 1255 amino acids and is identified by UniProt P15941 and shown in SEQ ID NO: 117. It is also known as MUC-1, Breast carcinoma-associated antigen DF3, Cancer antigen 15-3, CA 15-3, Carcinoma-associated mucin, Episialin, H23AG, KL-6, PEMT, Peanut-reactive urinary mucin (PUM), Polymorphic epithelial mucin (PEM), Tumor-associated epithelial membrane antigen (EMA), or Tumor-associated mucin and CD227. There are 17 isoforms of different length, whereby isoform 1 (UniProt: P15941-1, SEQ ID NO: 117) corresponds to the major form and has been chosen as canonical isoform in UniProt and which is used as reference isoform for the numbering of the protein (and the numbering of the other isoforms). Isoform Y (UniProt: P15941-Y, SEQ ID NO: 118) is a shorter form, which lacks amino acids 54-1053 of SEQ ID NO: 117 and is also implicated in cancer.

Mucin-1 has been described as a transmembrane protein consisting of 2 subunits that form a stable dimer. Mucin-1 has a signal peptide (amino acids 1-23 of SEQ ID NO: 117) and is autocatalytically cleaved at amino acid position 1097 of SEQ ID NO: 117. The MUC-1 amino terminal subunit (amino acids 24-1097 of SEQ ID NO: 117) contains variable numbers of tandem repeats that are modified by glycosylations. The number of tandem repeats can vary between different individuals. This subunit has been implicated in cell adhesion. The amino terminal subunit is attached to the membrane via non-covalent binding to the carboxy terminal subunit. The carboxy terminal subunit (amino acids 1098-1255 of SEQ ID NO: 117) consists of a 58 amino acid extracellular domain, a 28 amino acid transmembrane domain and a 72 amino acid cytoplasmic domain. Like the amino terminal subunit, the carboxy terminal subunit can also be glycosylated. The carboxy terminal peptide has been shown to play a role in cell signalling. The extracellular domain of MUC-1 can also be released into the extracellular matrix and thus shed into serum.

Mucin-1 is expressed in epithelial tissue layers lining ducts and lumens in different parts of the human body. Altered expression of MUC-1, has been implicated in cancer formation and metastasis. Both the amino and carboxy terminal subunits contribute to oncogenic activity. Mucin-1 is used as a serological clinical marker of breast cancer to monitor response to breast cancer treatment and disease recurrence, whereby increased levels may indicate disease progression.

Amino acids 24-1158 of SEQ ID NO: 117 comprise the extracellular domain of Mucin-1, which comprises the epitope of said antibody binding to Mucin-1. Most antibodies target the highly immunodominant core peptide domain of a 20 amino acid tandem repeat.

The structure and function of Mucin-1 is described, for example, in Duffy et al., 2010, Clin Chim Acta, 411(23-24):1869.

Information on Epidermal Growth Factor Receptor (EGFR) can be derived for example from Uniprot or suitable literature sources such as Sabbah et al., 2020, Curr Top Med Chem; 20(10):815-834.

Information on Her2 can be derived for example from Uniprot or suitable literature sources such Guitierrez and Schiff, 2011, Arch Pathol Lab Med, 135(1): 55-62.

The antibody or antigen binding fragment targeting MSLN, CEACAM5, B7H3, CA125, EGFR, Her2 or Mucin-1 may have any antibody format as described herein, preferably be an IgG antibody, more preferably an IgG1 or IgG4 antibody, or a Fab fragment or a scFv thereof. The antibody is preferably a monoclonal antibody, more preferably a humanized or human monoclonal antibody and the antigen binding fragment may be derived from these formats. The antibody or antigen binding fragment targeting MSLN, CEACAM5, B7H3, CA125, EGFR, Her2 or Mucin-1 may have an affinity for the respective targets of <20 nM.

In particular embodiments, the antibody or antigen-binding fragment thereof targeting Mesothelin (MSLN) is an antibody comprising a variable heavy chain complementarity determining region 1 (CDRH1) sequence of SEQ ID NO: 111; a variable heavy chain complementarity determining region 2 (CDRH2) sequence of SEQ ID NO: 112; a variable heavy chain complementarity determining region 3 (CDRH3) sequence of SEQ ID NO: 113; a variable light chain complementarity determining region 1 (CDRL1) sequence of SEQ ID NO: 114; a variable light chain complementarity determining region 2 (CDRL2) sequence of SEQ ID NO: 115; and a variable light chain complementarity determining region 3 (CDRL3) sequence of SEQ ID NOs: 116.

In further particular embodiments, the antibody or antigen-binding fragment thereof targeting Mesothelin (MSLN) is an antibody comprising a variable heavy chain complementarity determining region 1 (CDRH1) sequence of SEQ ID NO: 119 or SEQ ID NO 127; a variable heavy chain complementarity determining region 2 (CDRH2) sequence of SEQ ID NO: 120; a variable heavy chain complementarity determining region 3 (CDRH3) sequence of SEQ ID NO: 121; a variable light chain complementarity determining region 1 (CDRL1) sequence of SEQ ID NO: 122; a variable light chain complementarity determining region 2 (CDRL2) sequence of SEQ ID NO: 123; and a variable light chain complementarity determining region 3 (CDRL3) sequence of SEQ ID NOs: 124.

It is further preferred that said antibody or antigen-binding fragment thereof targeting Mesothelin (MSLN) comprises a variable light chain amino acid sequence of SEQ ID NO: 102 and a variable heavy chain amino acid sequence of SEQ ID NO: 103.

It is additionally preferred that said antibody or antigen-binding fragment thereof targeting Mesothelin (MSLN) comprises a variable light chain amino acid sequence of SEQ ID NO: 126 and a variable heavy chain amino acid sequence of SEQ ID NO: 125 or SEQ ID NO: 128.

In a further embodiment the antibody or antigen-binding fragment thereof targeting Mesothelin (MSLN) is Amatuximab or a fragment or derivative thereof. Amatuximab (development code MORAb-009) is a chimeric monoclonal antibody designed for the treatment of cancer. It was developed by Morphotek, Inc. Amatuximab is identified by its CAS No. 931402-35-6 and its FDA UNII Code No. 6HP0354G04.

In a further embodiment the antibody or antigen-binding fragment thereof targeting Mesothelin (MSLN) is Anetumab or a fragment or derivative thereof. Anetumab is monoclonal antibody directed against mesothelin. It is identified by its FDA UNII Code No. WVL0274BWW and its NCI META CUI CL563095.

In a further embodiment the antibody or antigen-binding fragment thereof targeting Mesothelin (MSLN) is h7D9.v3 or a fragment or derivative thereof. h7D9.v3 is a humanized anti-MSLN IgG1 antibody. Further information on the antibody can be derived, for example, from US 20120225013.

In a further embodiment the antibody or antigen-binding fragment thereof targeting Mesothelin (MSLN) is or BMS-986148 or a fragment or derivative thereof. BMS-986148 is a fully human IgG1 anti-mesothelin monoclonal antibody fused to tubulysin and used as an antibody drug conjugate (ADC). It is identified by its FDA clinical trial No: NCT02341625.

In further particular embodiments, the antibody or antigen-binding fragment thereof targeting CEACAM5 is SAR408377 or a fragment or derivative thereof. SAR408377 is an IgG1 monoclonal antibody. It is identified by its CAS No. 2349294-95-5.

In a further embodiment the antibody or antigen-binding fragment thereof targeting CEACAM5 is Labetuzumab or a fragment or derivative thereof. Labetuzumab is a humanized IgG1 monoclonal antibody. It is identified by its CAS No. 219649-07-7 and its FDA UNII Code No. EFE6X5M3UY.

In a further embodiment the antibody or antigen-binding fragment thereof targeting CEACAM5 is SGM-ch511 or a fragment or derivative thereof. SGM-ch511 is anti-CEA chimeric mAb and used in SGM-101. Further information may be derived from Gutowski et al., 2017, Surgical Oncology 26, 153-162.

In a further embodiment the antibody or antigen-binding fragment thereof targeting CEACAM5 is Cergutuzumab or a fragment or derivative thereof. Cergutuzumab is a human immunoglobulin G1 kappa (IgG1k) monoclonal antibody directed against carcinoembryonic antigen. It is identified by its FDA UNII Code No. 021F0511E8 and its NCI META CUI No. CL1412072.

In further particular embodiments, the antibody or antigen-binding fragment thereof targeting B7H3 is Enoblituzumab or a fragment or derivative thereof. Enoblituzumab is an Fc-optimized humanized IgG1K monoclonal antibody, also known as MGA271. It is identified by its CAS No. 1353485-38-7 and its FDA UNII Code No. M6030H73N9.

In further particular embodiments, the antibody or antigen-binding fragment thereof targeting B7H3 is MGC018 or a fragment or derivative thereof. MGC018 is a humanized immunoglobulin G1 (IgG1)/kappa monoclonal antibody conjugated through reduced interchain disulfides to the cleavable linker-duocarmycin payload, valine-citrulline-seco DUocarmycin hydroxyBenzamide Azaindole (vc-seco-DUBA), with potential antineoplastic activity. MGC018 is identified by its FDA UNII Code No. 2ZK3D69DQJ and its NCI META CUI No. CL951453.

In further particular embodiments, the antibody or antigen-binding fragment thereof targeting B7H3 is Omburtamab or a fragment or derivative thereof. Omburtamab is a monoclonal antibody that binds to an FG-loop-dependent conformation on the B7H3 molecule to interfere with its biological function. It is identified by its CAS No. 1895083-75-6 and its FDA UNII Code No. XQL6783S5T.

In further particular embodiments, the antibody or antigen-binding fragment thereof targeting B7H3 is MABX-9001 or a fragment or derivative thereof. MABX-9001 is a humanized IgG1 antibody-drug conjugate (ADC) that releases its payload DXd upon internalization by cancer cells. Further information may be derived from Bendell et al., 2020, Journal of clinical oncology, 38: 15, 3646.

In further particular embodiments, the antibody or antigen-binding fragment thereof targeting CA125 is Abagovomab or a fragment or derivative thereof. Abagovomab is a murine IgG1 monoclonal anti-idiotype antibody, containing a variable antigen-binding region that functionally mimics the three-dimensional structure of a specific epitope on CA125. It is identified by its CAS No. 792921-10-9 and its FDA UNII Code No. 3YK0326U7X.

In further particular embodiments, the antibody or antigen-binding fragment thereof targeting CA125 is Oregovomab or a fragment or derivative thereof. Oregovomab is a murine monoclonal antibody. It is identified by its CAS No. 213327-37-8 and its FDA UNII Code No. HX101E7L6S.

In further particular embodiments, the antibody or antigen-binding fragment thereof targeting Mucin-1 is BTH1704 or a fragment or derivative thereof. BTH1704 is a monoclonal antibody against Mucin-1. It is identified by its PDQ Open Trial Search ID No. 764610 and its UMLS CUI No. C3896895.

In further particular embodiments, the antibody or antigen-binding fragment thereof targeting Mucin-1 is mAb-AR20.5 or a fragment or derivative thereof. mAb-AR20.5 is a murine anti-MUC-1 monoclonal antibody, also known as BrevaRex. Used to induce a MUC-1 antigen specific immune response/vaccination. Further information may be derived from Bose and Mukherjee, 2020, Vaccines, 8, 659.

In further particular embodiments, the antibody or antigen-binding fragment thereof targeting Mucin-1 is TAB004, or a fragment or derivative thereof. TAB004 is a recombinant humanized immunoglobulin G4 kappa (IgG4k) monoclonal antibody. It is identified by its FDA UNII Code No. D40H7X1YZ0 and its NCI META CUI No. CL1378714. Further information may be derived from Bose and Mukherjee, 2020, Vaccines, 8, 659.

In further particular embodiments, the antibody or antigen-binding fragment thereof targeting Mucin-1 is 1B2, or a fragment or derivative thereof. Further information may be derived from Madsen et al., 2013, Immunopharmcol. Immunotoxicol., 35, 649-652.

In further particular embodiments, the antibody or antigen-binding fragment thereof targeting Mucin-1 is HMFG1 or a fragment or derivative thereof. HMFG1 is a humanized monoclonal antibody. It is identified by its PDQ Open Trial Search ID No. 43644 and its UMLS CUI No. C0879349.

In further particular embodiments, the antibody or antigen-binding fragment thereof targeting Mucin-1 is PankoMab or a fragment or derivative thereof. PankoMab is humanized monoclonal antibody, which is also known as Gatipotuzumab. It is identified by its CAS No. 1264737-26-9 and its FDA UNII Code No. 85V55ZLV2Z.

In further particular embodiments, the antibody or antigen-binding fragment thereof targeting Mucin-1 is KL-6 or a fragment or derivative thereof. Further information may be derived from Bose and Mukherjee, 2020, Vaccines, 8, 659.

In further particular embodiments, the antibody or antigen-binding fragment thereof targeting Mucin-1 is 5E5 or a fragment or derivative thereof. 5E5 is a mouse IgG1k mAb that was generated by immunization of wild-type Balb/c mice with GalNAc-glycosylated MUC-1 glycopeptide conjugated to KLH. Further information may be derived from Bose and Mukherjee, 2020, Vaccines, 8, 659.

In further particular embodiments, the antibody or antigen-binding fragment thereof targeting Mucin-1 is GGSK-1/30 or a fragment or derivative thereof. GGSK-1/30 is a monoclonal antibody selectively recognizing human tumor-associated MUC-1. This antibody targets exclusively tumor-associated MUC-1 in the absence of any binding to MUC-1 on healthy epithelial cells. Further information may be derived from Stergiou et al., 2019, Int J Med Sci, 16(9):1188-1198.

In further particular embodiments, the antibody or antigen-binding fragment thereof targeting EGFR is Cetuximab, Panitumumab, Nimotuzumab, Necitumumab, Depatuxizumab, Futuximab, Imgatuzumab, Matuzumab, GC1118, AMG595, Mab A13, MRG003, AVID100, SHR-A1307, RN765C, ABT-414, ABT-806 or ABBV-321 or a fragment or derivative thereof.

In further particular embodiments, the antibody or antigen-binding fragment thereof targeting Her2 is Pertuzumab, Trastuzumab, Margetuximab, MCLA-128, GBR 1302, RC48, DS-8201a, FS-1502, SYD985 or ARX788 or a fragment or derivative thereof.

Preferably, the pharmaceutical composition comprises suitable formulations of carriers, stabilizers and/or excipients. In a preferred embodiment, the pharmaceutical composition comprises a composition for parenteral, transdermal, intraluminal, intraarterial, intrathecal and/or intranasal administration or by direct injection into tissue. It is envisaged that said composition is administered to a patient via infusion or injection.

The term “pharmaceutically acceptable” as used herein means approved by a regulatory agency or other generally recognized pharmacopeia for use in animals, and more particularly in humans. The term “carrier” refers to a diluent, excipient, or pharmaceutical vehicle with which the protein construct is administered. Such a carrier is pharmaceutically acceptable, i. e. is non-toxic to a recipient at the dosage and concentration employed. It is preferably isotonic, hypotonic or weakly hypertonic and has a relatively low ionic strength, such as provided by a sucrose solution. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers. Suitable pharmaceutical excipients include starch, glucose, sucrose, gelatine, malt, rice, flour, chalk, silica gel, sodium stearate, glycerol monostearate, talc, sodium ion, dried skim milk, glycerol, propylene, glycol, water, ethanol and the like. The composition, if desired, can also contain minor amounts of wetting or emulsifying agents, or pH buffering agents. These compositions can take the form of, e. g., solutions, suspensions, emulsion and the like. Examples of suitable pharmaceutical carriers are described, for example, in “Remington's Pharmaceutical Sciences” by E. W. Martin. Generally, the ingredients may be supplied either separately or mixed together in unit dosage form. In a specific embodiment, the pharmaceutical composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Where the composition is to be administered by infusion, it can be dispensed with an infusion bottle containing sterile pharmaceutical grade water or saline, preferably comprising 0.9% NaCl. Where the composition is administered by injection, an ampoule of sterile water for injection or saline can be provided so that the ingredients may be mixed prior to administration.

The term “pharmaceutical adjuvant” as used herein relates to additional ingredients such as chloroquine, protic polar compounds, such as propylene glycol, polyethylene glycol, glycerol, EtOH, 1-methyl L-2-pyrrolidone or their derivatives, or aprotic polar compounds such as polysorbates (e.g. Tween®-20), dimethylsulfoxide (DMSO), diethylsulfoxide, di-n-propylsulfoxide, dimethylsulfone, sulfolane, dimethylformamide, dimethylacetamide, tetramethylurea, acetonitrile or their derivatives. The pharmaceutical adjuvant may further be one or more of a surfactant wetting agent, dispersing agent, suspending agent, buffer, stabilizer or isotonic agent. The present invention also envisages any suitable pharmaceutical adjuvant as known to the skilled person.

The pharmaceutical composition of the present invention can also comprise a preservative. Preservatives according to certain compositions of the invention include, without limitation: butylparaben; ethylparaben; imidazolidinyl urea; methylparaben; O-phenylphenol; propylparaben; quaternium-14; quaternium-15; sodium dehydroacetate; zinc pyrithione; and the like. The preservatives are used in amounts effective to prevent or retard microbial growth. Generally, the preservatives are used in amounts of about 0.1% to about 1% by weight of the total composition with about 0.1% to about 0.8% being preferred and about 0.1% to about 0.5% being most preferred.

The composition of the present invention can be administered to a subject or patient. The term “subject” or “patient” refers to a mammal. “Mammal” as used herein is intended to have the same meaning as commonly understood by one of ordinary skill in the art. Preferred mammals are primates, cows, sheep, goats, horses, dogs, cats, rabbits, rats, mice and the like. In particularly preferred embodiments, the subject is a human.

The term “administered” means administration of a therapeutically effective dose of the pharmaceutical composition by any suitable route. By “therapeutically effective amount” is meant a dose that produces the effects for which it is administered in a patient. The exact dose will depend on the purpose of the treatment, and will be ascertainable by one skilled in the art using known techniques. As is known in the art and described herein, adjustments for systemic versus localized delivery, age, body weight, general health, sex, diet, time of administration, drug interaction and the severity of the condition may be necessary, and will be ascertainable with routine experimentation by those skilled in the art. Administration of the composition may be effected in different ways, e. g., intravenously, intraperitoneally, subcutaneously, intramuscularly, topically or intradermally. In certain embodiments, the present invention provides for an uninterrupted administration of the composition comprising the protein construct. In an example, uninterrupted, i. e. continuous administration may be realized by a pump system. A subcutaneous administration may include a needle or a cannula for penetrating the skin of a patient and delivering the suitable composition into the patient's body. The administration may further be transdermal by way of a patch worn on the skin and replaced at intervals.

In a further aspect the present invention relates to the anti-CD47 antibody or antigen-binding fragment including constructs comprising the anti-CD47 antibody or an antigen-binding fragment thereof and a further functional domain as defined above, the product as defined above, or the pharmaceutical composition as mentioned above for use in the treatment of cancer. Also envisaged is a method for the treatment of cancer, wherein said method comprises administering to a patient in need thereof the anti-CD47 antibody or antigen-binding fragment including constructs comprising the anti-CD47 antibody or an antigen-binding fragment thereof and a further functional domain, the product as defined above, or the pharmaceutical composition as mentioned above.

The term “treatment”, unless otherwise indicated by context, refers to therapeutic treatment and/or prophylactic measures to prevent the outbreak or relapse of a disease or pathological condition, wherein the objective is to inhibit or slow down (lessen) an undesired physiological condition. For purposes of this invention, beneficial or desired clinical results include, but are not limited to, alleviation of symptoms, diminishment of extent of disease, stabilized (i. e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already having the condition or disorder as well as those prone to have the condition or disorder. The treatment may further, in specific embodiments, involve a single administration of a pharmaceutical composition, protein construct or product as defined above, or multiple administrations. A corresponding administration scheme may be adjusted to the sex or weight of the patient, the disease, the pharmaceutical composition to be used, the general health status of the patient etc. For example, the administration scheme may contemplate an administration every 12 h, 24 h, 28 h, 72 h, 96 h, once a week, once every two weeks, once every 3 weeks, once a month etc. Also envisaged are pauses or breaks between administration phases. These regimens can of course be adjusted or changed by the medical practitioner in accordance with the patient's reaction to the treatment and/or the course of disease or of the pathological condition.

The term “cancer” as used herein relates to a pathological process that results in the formation and growth of a cancerous or malignant neoplasm, i. e., abnormal tissue that grows by cellular proliferation, often more rapidly than normal and continues to grow after the stimuli that initiated the new growth cease. Malignant neoplasms typically show partial or complete lack of structural organization and functional coordination with the normal tissue and most invade surrounding tissues, metastasize to several sites, and are likely to recur after attempted removal and to cause the death of the patient unless adequately treated. The term thus also includes the existence and development of metastases. As used herein, the term “neoplasia” is used to describe all cancerous disease states and embraces or encompasses the pathological process associated with malignant hematogenous, ascitic and solid tumors. Representative cancers include, for example, stomach, gastric, colon, oesophagus, rectal, liver, pancreatic, lung, breast, including triple negative breast cancer, cervix uteri, corpus uteri, ovary, prostate, including metastatic prostate cancer, testis, bladder, renal, head and neck, throat cancer, ascites, mesothelioma, melanoma skin cancer, non-melanoma skin cancer, Hodgkin's disease, non-Hodgkin's lymphoma, multiple myeloma, leukemia, acute lymphocytic leukemia, acute myelogenous leukemia, and lymphoma. Also envisaged are further cancer forms known to the skilled person or derivable from suitable literature sources such as Pavlopoulou et al., 2015, Oncol Rep., 33, 1, 3-18.

It is particularly preferred that the cancer is ovarian cancer, ascites, mesothelioma, breast cancer, triple negative breast cancer, pancreatic cancer, pancreatic adenocarcinoma, non-small cell lung cancer (NSCLC), colorectal cancer, endometrial cancer, biliary extrahepatic cancer, lymphoma, non-hodgkin lymphoma (NHL), diffuse large B cell lymphoma (DLBCL), leukemia, acute myeloid leukemia and myelodysplastic syndrome (MDS).

The cancer may, in certain embodiments, be a refractory cancer. A cancer may be assumed to be residually present if a subject has undergone surgery, chemotherapy or radiotherapy as previous treatment for the cancer. Also envisaged are metastasizing cancer forms, e. g. of the above-mentioned cancer forms.

The following examples and figures are provided for illustrative purposes. It is thus understood that the example and figures are not to be construed as limiting. The skilled person in the art will clearly be able to envisage further modifications of the principles laid out herein.

### EXAMPLES

### Example 1

### Generation and Purification of Anti-CD47 mAb

To obtain new anti-human CD47 antibodies with a new binding sequence, specifically with new complementary determining regions (CDRs), mice were immunized with the recombinantly expressed extracellular domain of human CD47. The immunization was followed by selection of hybridoma clones and anti-CD47 antibodies that bound human CD47 by enzyme-linked immunosorbent assay (ELISA), surface plasmon resonance (SPR) and flow cytometry and prevented SIRPalpha binding to CD47 in a competitive manner by flow cytometry or SPR were selected.

The selected antibodies were further tested for their binding to CD47-expressing tumor cell lines, such as Expi293™ cells and Molm-13 cancer cells. The variable fragment (Fv) of the selected antibodies was then sequenced from the hybridoma cells and the CDRs and framework amino acids were determined.

FIG. 1 shows schematic examples of a monoclonal antibody (mAb) and possible fragments and antigen-binding fragments thereof. Examples detailed below are showing data obtained with anti-CD47 IgG4, anti-CD47 IgG1, anti-CD47 Fab and anti-CD47 scFv. Example 6, below, provides data obtained with an anti-CD47 scFv-anti-mesothelin IgG1.

Humanized anti-CD47 mAb were generated by grafting the CDR sequences from variable light (VL) and heavy chains (VH) of the anti-hCD47 antibodies into human consensus frameworks derived from a publicly available list of humanized therapeutic antibodies (template antibody sequences). Sequences were selected based on: sequence similarity between VL or VH and the template antibody sequences, CDR length and optimal pairing preferences between VL and VH human germlines. Key amino acids were determined independently for each humanization using publicly available PDB structures of the template antibodies and were retained in the humanized antibodies.

Point mutations on the amino acid level were inserted for affinity reduction. For example, amino acids were back-mutated to the amino acid found in the germline sequence, or changed to an amino acid with similar characteristics, or changed into an amino acid with a completely different characteristic.

The desired VL and VH sequences were generated using custom gene synthesis. The VL was subcloned into the pFUSE2-CLIg-hk vector and the VH into the pFUSE-CHIg-hG1, pFUSE-CHIg-hG4, or pSecTag vector. All vectors used contain a leader sequence or signal peptide for protein secretion to the cell culture supernatant.

The corresponding plasmids containing the coding sequence of the vector backbone and the respective anti-hCD47 antibody sequence were transiently transfected and expressed in Expi293F™ cells and analysed for their expression and binding after purification.

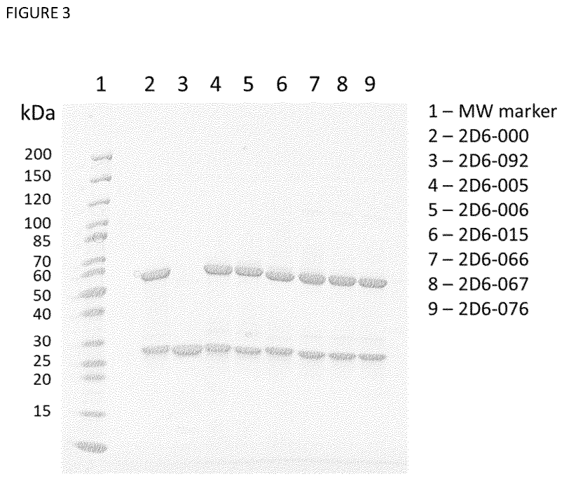

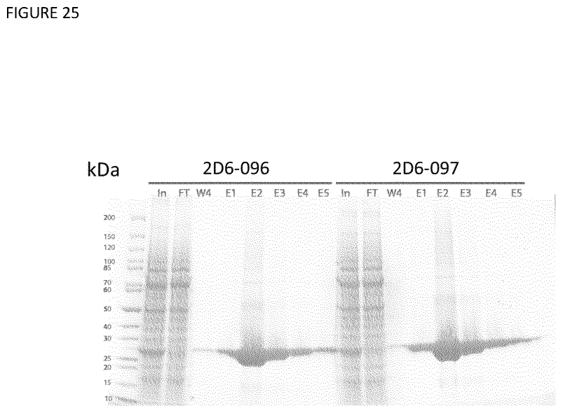

After five to seven days, the cell culture supernatant was harvested and proteins were purified by protein A affinity chromatography and acidic elution (pH2.5-3.5) followed by neutralization of the pH. Size exclusion chromatography (SEC) of the purified molecules was performed using a Superdex® 200 increase 10/300 column in phosphate buffered saline (PBS) and the proteins were analyzed by 4-20% sodium dodecylsulfat polyacrylamide gel electrophoresis (SDS-PAGE) under reducing conditions and visualized by Coomassie® Brilliant Blue staining (see FIG. 3 and FIG. 25).

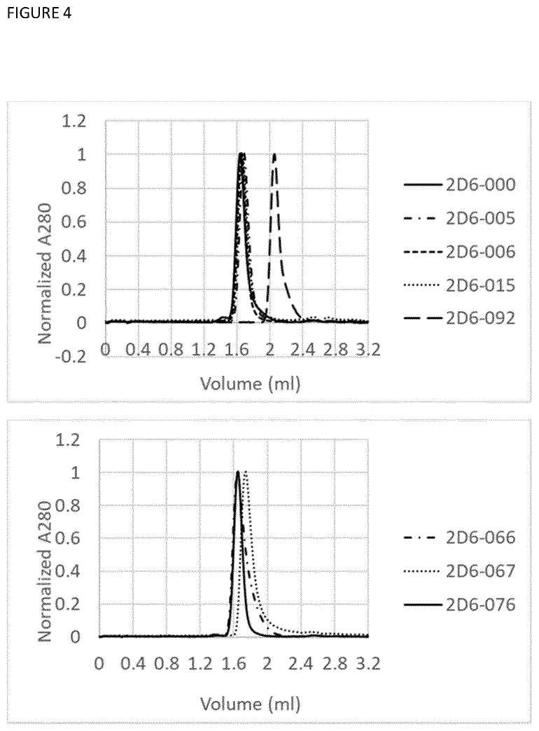

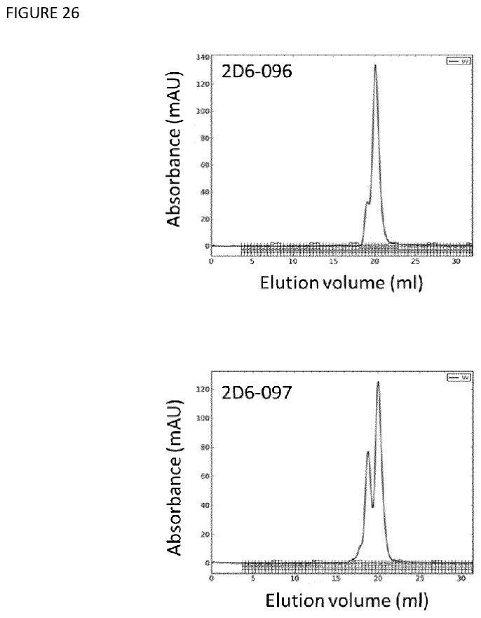

All antibodies were expressed and could be purified to at least 95% purity as shown on the analytical SEC (see FIG. 4) and used for subsequent functional assays. Anti-CD47 scFvs could be purified as standalone polypeptides but to a lower percentage of purity as shown on the analytical SEC profile depicted in FIG. 26.

### Example 2

### The Anti-hCD47 Antibodies and Fragments Thereof Bind to CD47

Initially, binding of anti-hCD47 IgG4, anti-hCD47 IgG1 antibodies and Fab domains were measured on CD47-expressing cells by flow cytometry. Cells were stained with 50 nM of antibody followed by staining with a secondary fluorescently labelled anti-human IgG Fc or light-chain antibody, and analyzed by flow cytometry. Binding was measured by calculating the median fluorescence intensity (MFI) ratio, by dividing MFI of the antibody by the MFI of the isotype control.

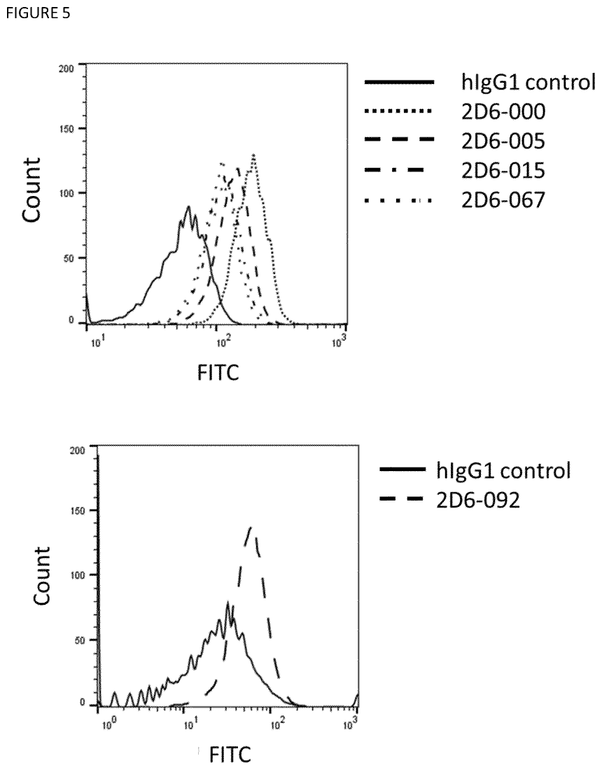

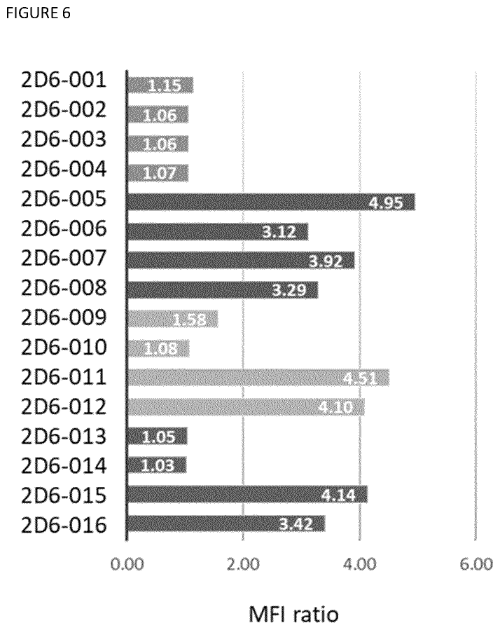

FIG. 5 shows examples of binding measured by flow cytometry on CD47-positive Molm-13 tumor cells, whereas FIG. 6 shows examples of binding measured by flow cytometry on CD47-posivite Expi293™ cells.

Surface Plasmon Resonance (SPR) was used in addition to analyse binding of the different protein constructs to recombinantly expressed CD47 and to determine their affinity. Association (Kₒₙ) and dissociation (K_{off}) rate constants were determined by SPR using a Biacore™ X-100 (Cytiva). Using the Human antibody capture kit an anti-human Fc antibody was immobilized on a CM5 sensor chip in a covalent manner using the Amine coupling Kit according to manufacturer's instructions. Humanized anti-hCD47 IgGs were injected as ligand to reach a surface coverage of approximately 100 RU-400 RU. Recombinantly expressed human CD47 extracellular domain was used as analyte and injected in increasing concentrations (e. g. from 3.9 to 1000 nM), with standard association time of 120 s and dissociation time of 600 s. Experiments were performed in multi-cycle kinetics runs, with regeneration between sample injection with the respective regeneration buffer (3M MgCl2 for the human antibody capture kit). All reagents were diluted in HBS-EP+ buffer (Cytiva). Data are fit to a simple 1:1 interaction model using the global data analysis option available within BiaEvaluation 2.0.1 software. The dissociation constant (KD) was used as a value for the affinity and was calculated from the ratio between K_{off} and Kₒₙ.

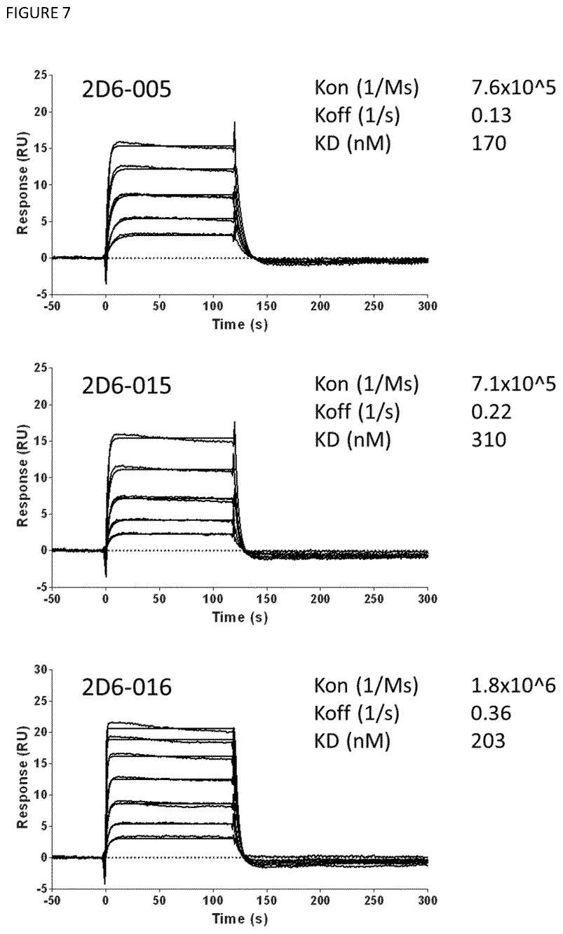

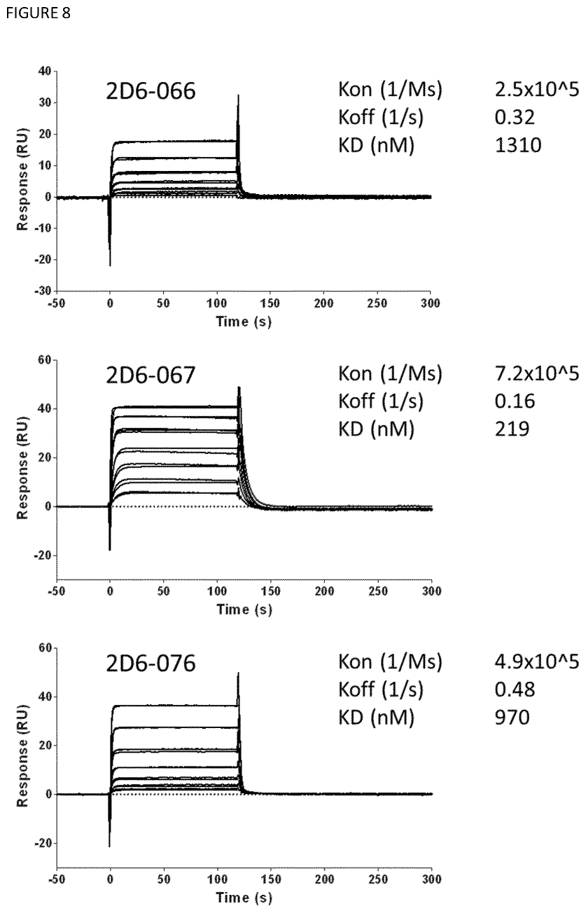

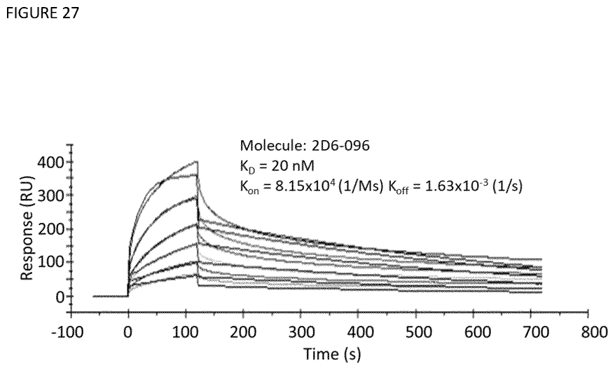

FIGS. 7 and 8 show examples of binding affinities measured by SPR for IgG4 and IgG1, respectively. FIG. 27 exemplarily illustrates the binding of a scFv by SPR. An overview of the affinity of the different antibody constructs for CD47 obtained by SPR, and expressed as KD, is shown in Table 5 below.

TABLE 5
Binding affinity of different antibody constructs measured by
SPR using a Biacore ™ X100 (Cytiva). The antibody constructs
differ in their sequences for CD47 binding or construct type
or specificity for a tumor antigen. TAA: Tumor associated antigen.

| Construct | | KD measured |
|---|---|---|
| name | Construct type | by SPR (nM) |
| 2D6-000 | IgG4 | 56.12 |
| 2D6-005 | IgG4 | 176.2 |
| 2D6-015 | IgG4 | 310.8 |
| 2D6-016 | IgG4 | 203.3 |
| 2D6-022 | Anti-CD47-scFv-anti-TAA IgG1 | 106.4 |
| 2D6-031 | Anti-CD47-scFv-anti-TAA IgG1 | 137.6 |
| 2D6-032 | Anti-CD47-scFv-anti-TAA IgG1 | 140.8 |
| 2D6-036 | Anti-CD47-scFv-anti-TAA IgG1 | 1113 |
| 2D6-037 | Anti-CD47-scFv-anti-TAA IgG1 | 176.2 |
| 2D6-046 | Anti-CD47-scFv-anti-TAA IgG1 | 406.0 |
| 2D6-066 | IgG1 | 1315 |
| 2D6-067 | IgG1 | 218.6 |
| 2D6-076 | IgG1 | 973.5 |
| 2D6-096 | scFv | 20 |
| MSL-247 | Anti-CD47-scFv-anti-MSLN IgG1 | 459.0 |
| MSL-248 | Anti-CD47-scFv-anti-MSLN IgG1 | 460.0 |
| MSL-274 | Anti-CD47-scFv-anti-MSLN IgG1 | 1315 |
| MSL-253 | Anti-CD47-scFv-anti-MSLN IgG1 | 399 |
| MSL-741 | Anti-CD47-scFv-anti-MSLN IgG1 | 537 |
| MSL-742 | Anti-CD47-scFv-anti-MSLN IgG1 | 537 |
| MSL-745 | Anti-CD47-scFv-anti-MSLN IgG1 | 578 |

### Example 3

Anti-CD47 Antibodies and Fragments Thereof are Able to Block the Interaction of CD47 with SIRPalpha

In order to inhibit the CD47 immune checkpoint, the protein constructs not only need to bind CD47, but they also need to block the interaction of CD47 with its receptor SIRPalpha. This was initially assessed by flow cytometry. CD47-positive Molm-13 tumor cells were incubated with 500 nM of anti-CD47 mAbs, a Fab fragment or scFvs, or control antibodies for 30 min at room temperature (RT). Without washing, a fluorescently labelled SIRPalpha construct was subsequently added to the cells, and incubated for an additional 30 min at RT. The signal was subsequently measured by flow cytometry. The fluorescently labelled SIRPalpha construct binds only to CD47 sites that are not already occupied by the anti-CD47 mAbs or fragments thereof. A decrease in signal intensity therefore corresponds to blocking of CD47 by the respective protein construct.

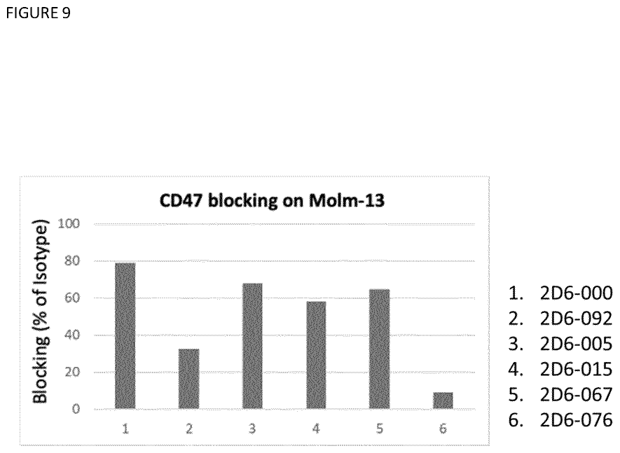

In FIG. 9 the percentage of blocking is shown for different constructs, demonstrating that the anti-hCD47 IgG protein constructs of the present invention and fragments thereof (e. g. Fab) are indeed able to block the CD47-SIRPalpha immune checkpoint interaction.

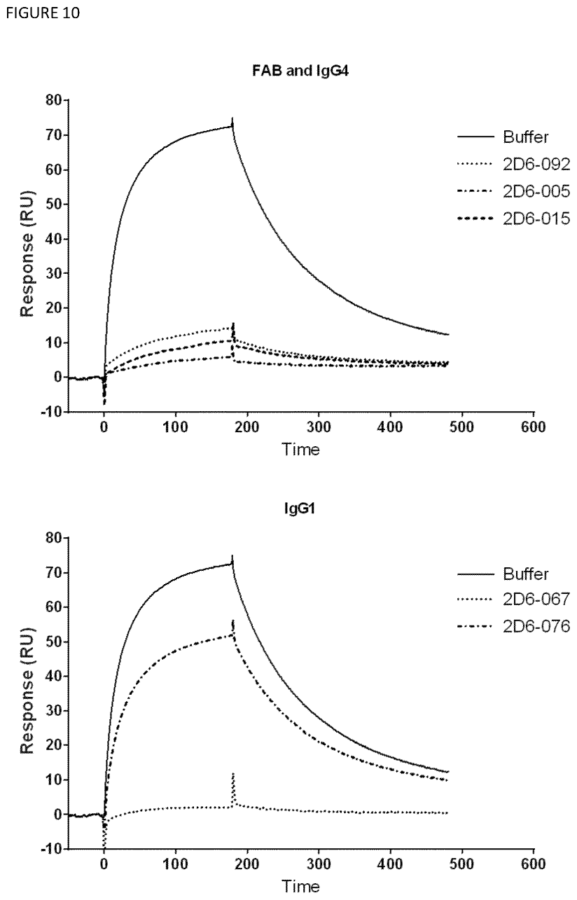

Another way to assess this is using a CD47-SIRPalpha competitive binding assay by SPR. A hSIRPalpha-mouse Fc fusion construct was used and coupled as ligand onto an anti-mouse Fc sensor chip and a hCD47ex (extracellular domain of human CD47)-human Fc fusion construct was used as analyte. Increasing concentrations of anti-CD47-IgG1, anti-CD47-IgG4, anti-CD47 Fab or anti-CD47 scFv were then mixed together with a fixed concentration of hCD47ex-hFc, resulting in a dose-dependent reduction of SPR signal, consistent with an inhibition of the binding between hSIRPalpha-mFc and hCD47ex-hFc (see FIG. 10).

### Example 4

### Anti-CD47 and Fragments Thereof are Able to Eliminate Tumor Cells by Phagocytosis

To test the ability of the anti-CD47 mAbs and fragment thereof e. g. scFv to kill tumor cells, an in vitro antibody-dependent cellular phagocytosis (ADCP) assay was performed using the CD47-positive tumor cell lines HL-60 as target cells and monocyte-derived macrophages as immune effector cells. Monocytes were isolated from healthy donors and differentiated to macrophages for 5 to 7 days in presence of human M-CSF. On the day of the experiment, macrophages (effector cells) were labelled with Calcein-AM, and the CD47-positive tumor cell lines HL-60 or other target cells (e.g. OVCAR3 tumor cells) were labelled with Calcein-Red-Orange. Macrophages and target cells were incubated at an effector-to-target cell ratio of 1:1 for 3 h together with saturating antibody concentrations (above 10 nM). The percentage of phagocytosis was analysed by flow cytometry and depicted as the ratio between double-positive macrophages (Calcein AM⁺ and Calcein-Red-Orange⁺) and total macrophages (Calcein AM⁺).

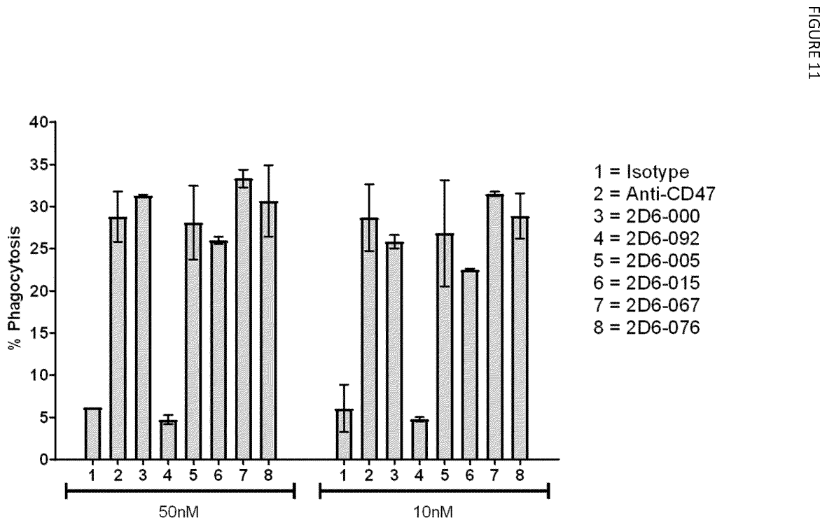

It could be confirmed that the anti-CD47 antibodies can induce phagocytosis of CD47 positive tumor cells (see FIG. 11), whereas, the Fab did not induced phagocytosis most likely due to the lack of a functional Fc domain.

### Example 5

Anti-CD47 scFv Fused to a Tumor Targeting IgG1

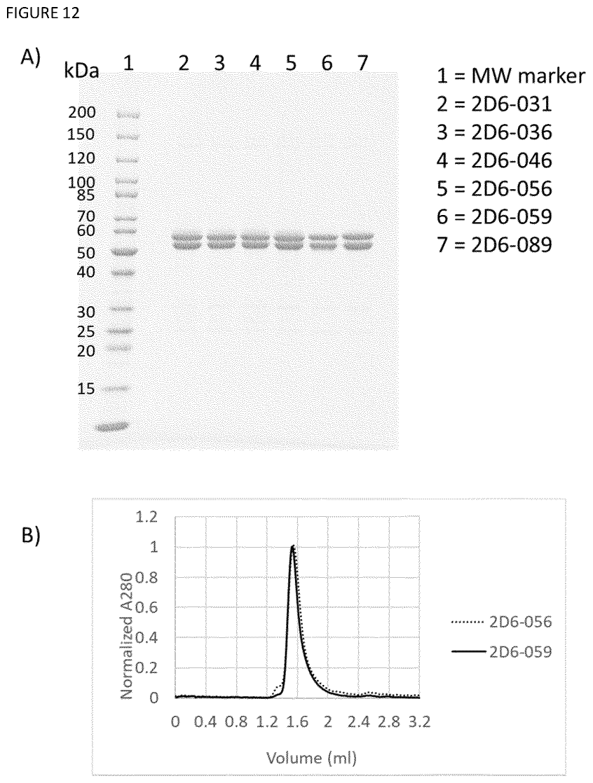

To show that antigen binding fragments of the low affinity anti-CD47 antibodies of the present invention can be combined and linked with an additional functional component different anti-CD47 scFv were fused to a tumor associated antigen (TAA) targeting IgG1. For this purpose, low affinity anti-CD47 scFv of the instant specification were synthesized using custom gene synthesis and subcloned together with a glycine/serine-rich polypeptide linker into the N-terminus of the light chain of an IgG1 targeting a TAA. Anti-CD47 scFvs were generated by subcloning the VH and the VL domains of an anti-CD47 antibody connected by the glycine/serine linker in a single polypeptide. The corresponding plasmids were transiently transfected into Expi293F™ cells for protein expression. These proteins were expressed and could be purified to at least 95% purity as shown on an SDS-PAGE and an analytical SEC (see FIG. 12) and used for subsequent functional assays.

Binding to CD47 was demonstrated using SPR (see FIG. 13), as described above.

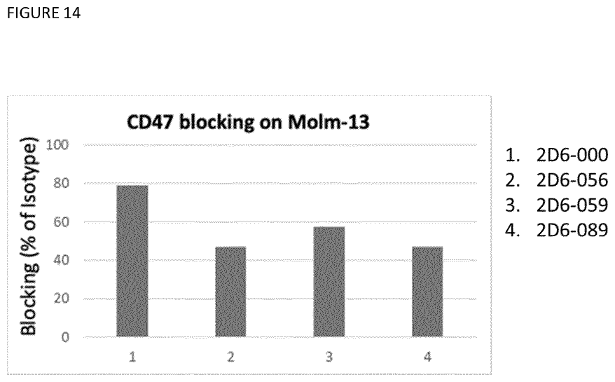

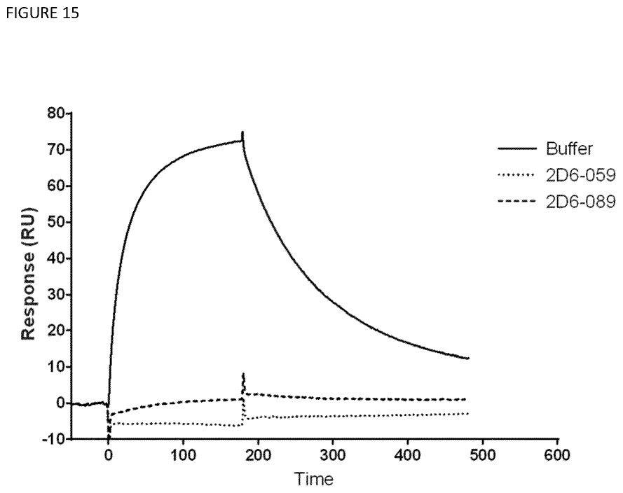

When fused to a TAA targeting IgG1 the anti-CD47 scFv was also able to block the CD47-SIRPalpha interaction on CD47-expressing MOLM-13 tumor cells. FIG. 14 shows the percentage of blocking obtained with the different constructs, an anti-CD47 IgG4 antibody (2D6-000) served as positive control. Blocking of the CD47-SIRPalpha interaction by the anti-CD47 scFv fused to an IgG1 targeting a TAA was confirmed also by SPR as depicted in FIG. 15.

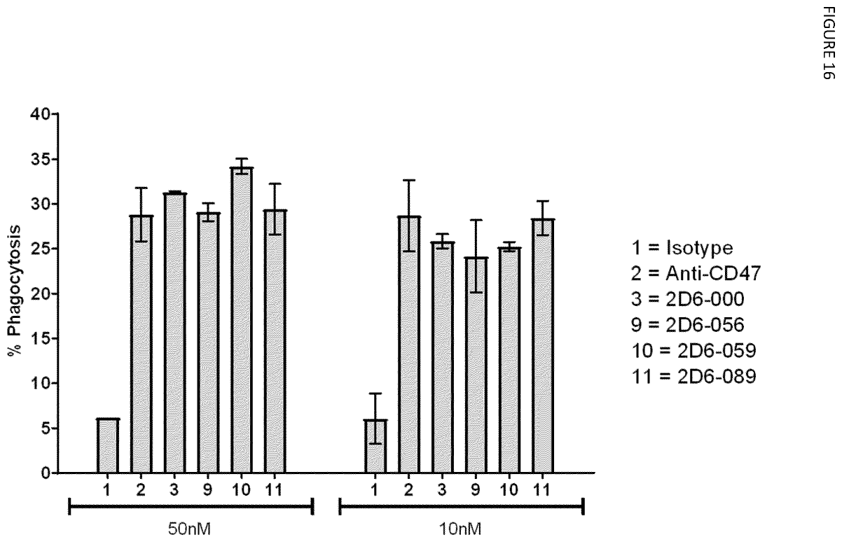

An ADCP assay with CD47 positive HL-60 cells was performed to show that the inhibition of the CD47-SIRPalpha interaction by the anti-CD47 scFv fused to a TAA targeting IgG1 construct can lead to the effective elimination of tumor cells (see FIG. 16).

### Example 6

Anti-CD47 scFv-Anti-MSLN Antibody/Protein Constructs

To show that antigen binding fragments of the low affinity anti-CD47 antibodies of the present invention can be combined and linked with different IgG1 mAbs binding a tumor associated antigen (TAA) expressed at the surface of a tumor cell, anti-CD47 scFv-anti-MSLN protein constructs were generated. Here, mesothelin (MSLN) serves as TAA or tumor marker. Anti-CD47 scFv of the instant specification were synthesized using custom gene synthesis and subcloned together with a glycine/serine-rich polypeptide linker onto the N-terminus (MSL-247, MSL-248, MSL-253, MSL-741, MSL-742, MSL-745) or the C-terminus (MSL-274) of the light chain of an anti-Mesothelin IgG1 mAb.

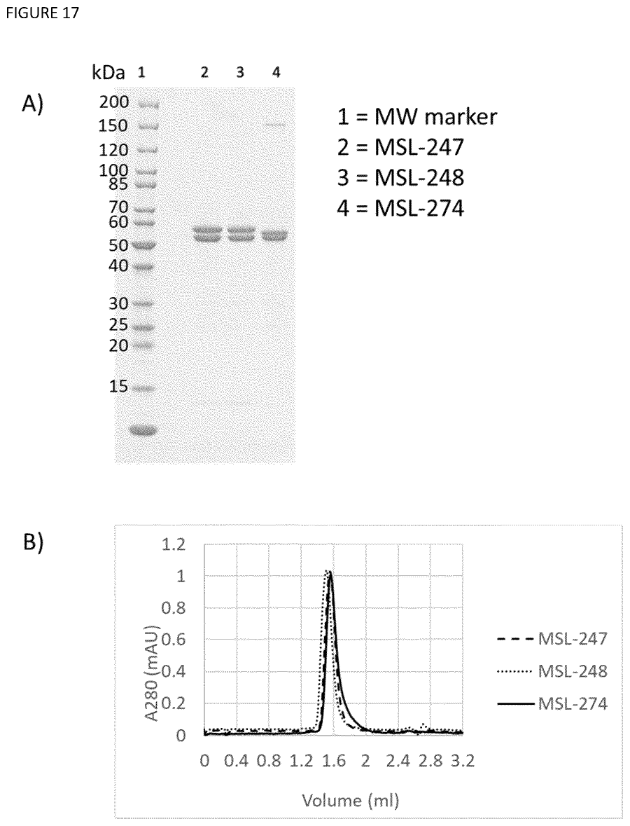

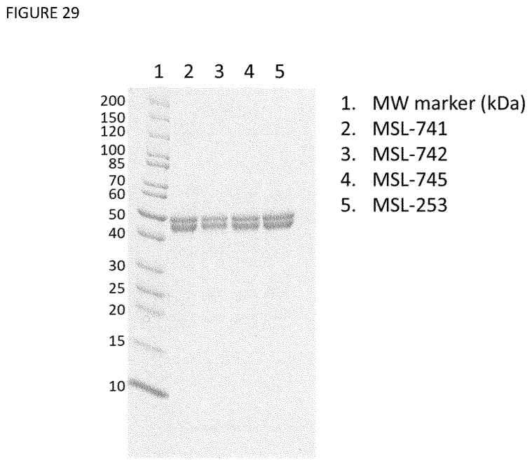

The proteins could be expressed and purified as shown in FIG. 17 and FIG. 29 by SDS PAGE and analytical size exclusion chromatography (aSEC).

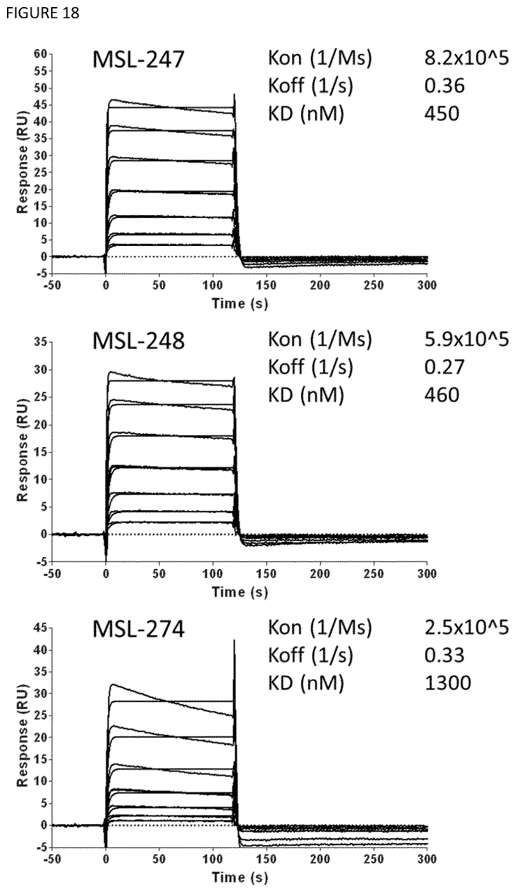

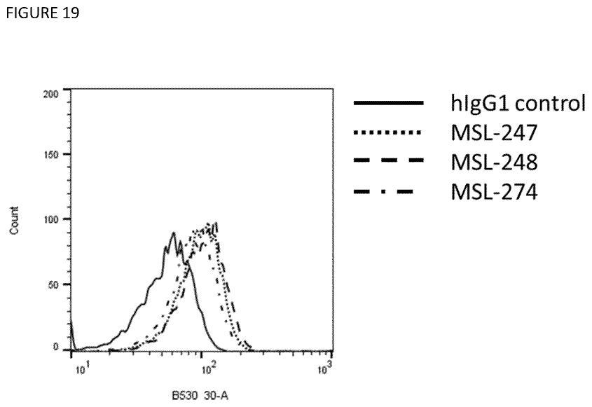

Binding to CD47 was demonstrated using SPR (see FIG. 18 and FIG. 28) and flow cytometry on mesothelin-negative, CD47 positive (MSLN−/CD47+) Molm-13 tumor cells (see FIG. 19).

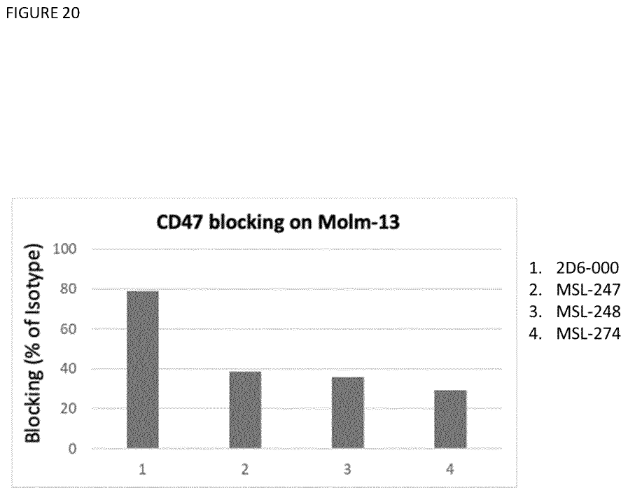

FIG. 20 shows that the anti-CD47 scFv-anti-MSLN not only bound to MSLN−/CD47+ Molm-13 tumor cells, but also blocked the CD47-SIRPalpha interaction on these cells, although to a somewhat lower degree than a high affinity anti-CD47 IgG4 (2D6-000).

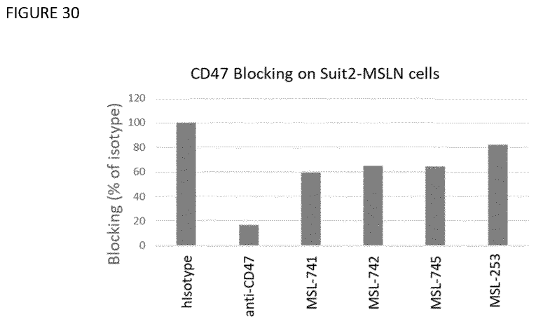

In FIG. 30 the percentage of CD47 blocking is shown by flow cytometry for different anti-CD47 scFv-anti-MSLN constructs using MSLN⁺/CD47⁺ double positive Suit-2 MSLN cells. This data clearly shows the ability of the protein constructs of the present invention and fragments thereof to block the CD47-SIRPalpha interaction and inhibit the CD47-SIRPalpha immune checkpoint.

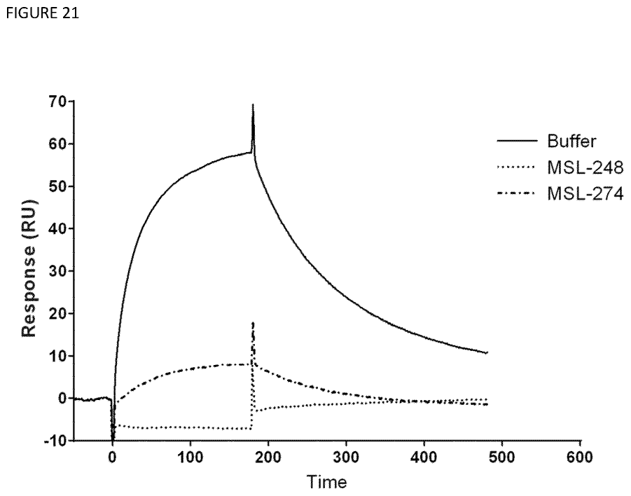

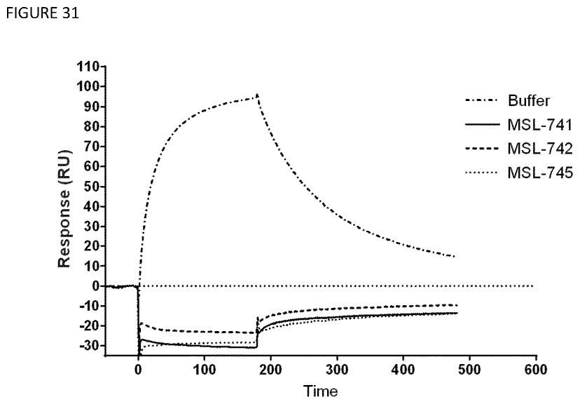

The blocking of the CD47-SIRPalpha interaction by the anti-CD47 scFv-anti-MSLN could also be confirmed by SPR (see FIG. 21 and FIG. 31). A hSIRPalpha-mouse Fc fusion construct was used and coupled as ligand onto an anti-mouse Fc sensor chip and a hCD47ex (extracellular domain of human CD47)-human Fc fusion construct was used as analyte. Increasing concentrations of anti-CD47 scFv-anti-MSLN protein constructs (MSL-741, MSL-742, MSL-745) were then mixed together with a fixed concentration of hCD47ex-hFc, resulting in a dose-dependent reduction of SPR signal, consistent with an inhibition of the binding between hSIRPalpha-mFc and hCD47ex-hFc.

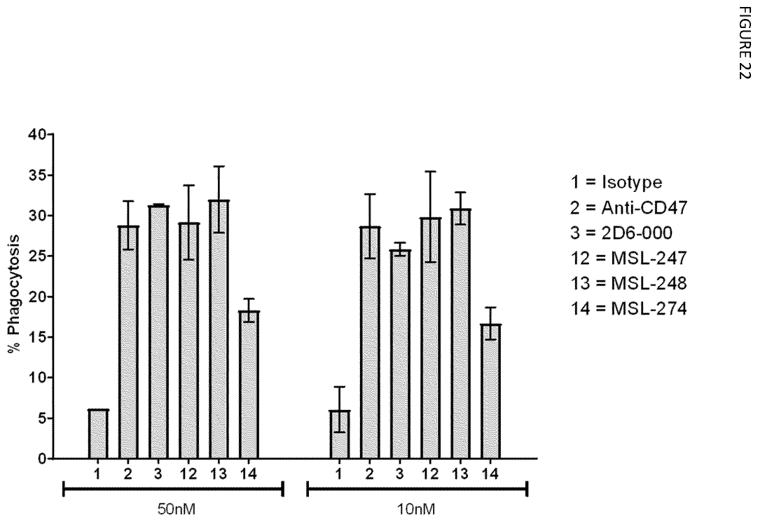

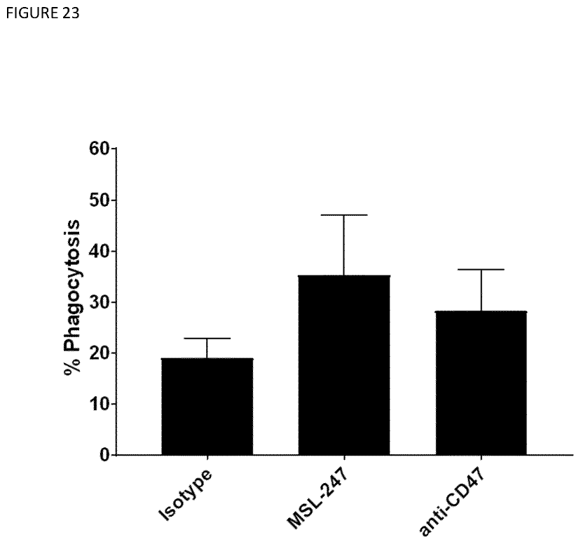

In order to show that this weaker inhibition of the CD47-SIRPalpha interaction by the anti-CD47 scFv-anti-MSLN construct has functional consequences and can lead to the elimination of tumor cells, ADCP assays were performed with MSLN−/CD47+ HL-60 tumor cells, but also with OVCAR-3 tumor cells positive for both CD47 and MSLN (MSLN+/CD47+). As shown in FIG. 22, the anti-CD47 scFv-anti-MSLN constructs induced similar phagocytosis as the high affinity anti-CD47 IgG4 using MSLN−/CD47+ HL-60 tumor cells as target cells. When MSLN+/CD47+ OVCAR-3 tumor cells were used, the anti-CD47 scFv-anti-MSLN construct induced even higher phagocytosis as the high affinity anti-CD47 due to the simultaneous targeting of two receptors (CD47 and MSLN) on the same target cells (see FIG. 23).

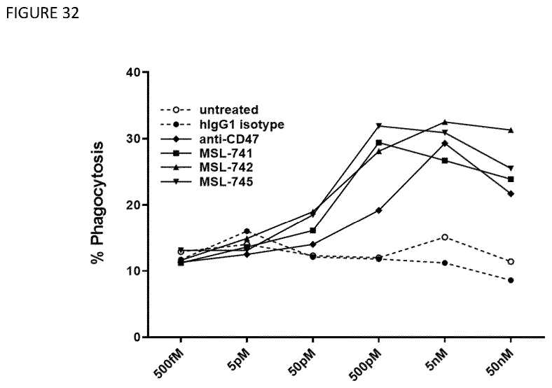

The ability of anti-CD47 scFv-anti-MSLN constructs according to the present invention has further been analysed on the tumor cell line OVCAR-3 in a concentration dependent manner (see FIG. 32). For the ADCP assay with the MSLN⁺/CD47⁺ double positive tumor cell line OVCAR-3 (target cells) monocytes were first isolated from the peripheral blood of healthy donors and subsequently differentiated to macrophages for 5 to 7 days in presence of 100 ng/ml recombinant hM-CSF. On the day of the experiment, macrophages (effector cells) were labelled with CellTrace Calcein red/orange AM, and MSLN⁺/CD47⁺ double positive OVCAR-3 tumor cells was labelled with CFSE. Macrophages and target cells were incubated at an effector-to-target (E:T) ratio of 1:1 for 2 h or 3 h in the presence of 500 fM to 50 nM of the respective protein constructs. Macrophages were detached using 1 mM EDTA in 1×PBS and optionally incubated for additional 1 h. Phagocytosis was analysed by flow cytometry and depicted as the ratio between phagocytosing double positive macrophages (CFSE⁺/Calcein red/orange AM⁺) and total macrophages (Calcein red/orange AM⁺).

In FIG. 32 it could be confirmed that the anti-CD47 scFv-anti-MSLN constructs can induce phagocytosis of MSLN⁺/CD47⁺ tumor cells in a concentration dependent manner.

### Example 7

### Anti-CD47 Antibodies and Fragments Thereof are Specific for CD47

The anti-CD47 antibodies and fragments thereof of the present invention have a low affinity to CD47 compared to standard monoclonal antibodies. This low affinity could correlate with low specificity. In order to assess if the anti CD47 antibodies and fragments thereof of the present invention bind to other targets than to CD47, binding to CD47-positive OVCAR-3 wild type (WT) tumor cells was assessed by flow cytometry and compared to binding to CD47-negative knockout (KO) OVCAR-3 cells. CD47 knockout OVCAR-3 cells were generated by CRISPR-CAS technology. Wild type OVCAR-3 cells expressing CD47 were transiently transfected with the PX458 (REF) plasmid, encoding for the selection marker green fluorescent protein (GFP), the Cas9 protein from S. pyogenes and a guide RNA targeting exon 1 of the human CD47 gene. GFP-positive cells were FACS-sorted and limiting dilutions were prepared to obtain single cell clones. CD47 KO clones were selected based on absent cell surface staining for the CD47 protein by flow cytometry.

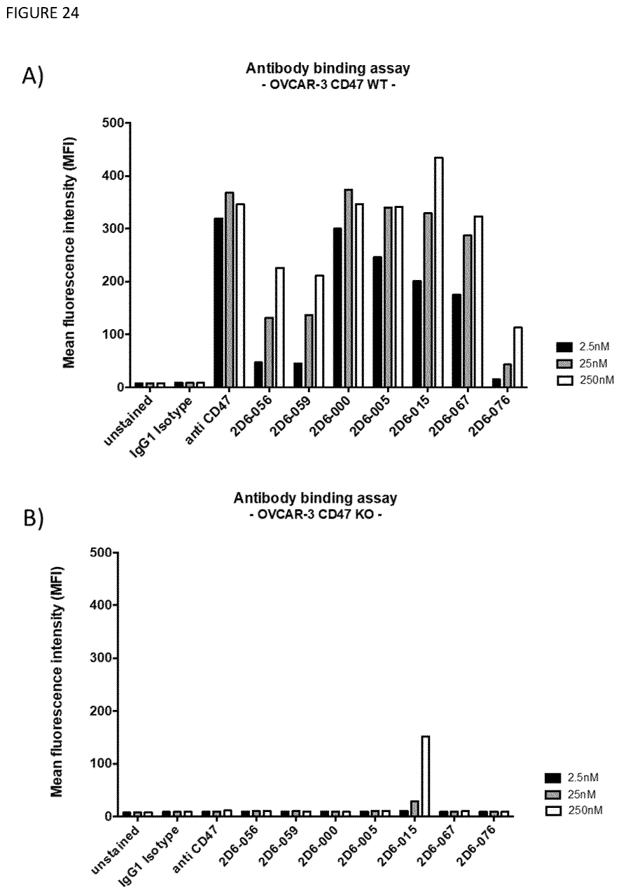

FIG. 24 shows that except the anti-CD47 antibody 2D6-015 all tested antibodies bind to CD47 expressing WT OVCAR-3 tumor cells, but not to KO OVCAR3 tumor cells in which CD47 was eliminated thus confirming that the anti-CD47 antibodies and fragments thereof of the present invention maintain a high specificity for CD47 despite their low affinity.

To further test the binding specificity to CD47 despite the low affinity of the anti-CD47 antibodies and fragments thereof such as anti-CD47 scFv, binding to wildtype (WT) Chinese ovarian hamster (CHO) cells not expressing human CD47 and CHO cells that were stably transfected with human CD47 (CD47⁺) was analyzed by flow cytometry.

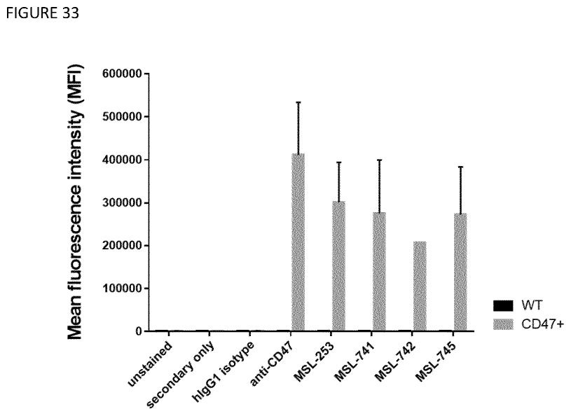

FIG. 33 shows the binding of anti-CD47 scFv-anti-MSLN constructs to WT CHO and CD47⁺ CHO cells, respectively, confirming that the anti-CD47 antibodies and fragments thereof still retain a high specificity for CD47, although they have a low affinity.

### Example 8

### Binding of Anti-CD47 Antibodies and Fragments Thereof to Red Blood Cells (RBCs)

As marker of self, CD47 is found on all cells in the body, including red blood cells (RBCs) or erythrocytes. Targeting CD47 with an hIgG1 antibody can be a major concern. To determine if the anti-CD47 scFv-anti-MSLN protein constructs bind to cells that express only CD47 but not MSLN, such as RBCs, binding of the anti-CD47 scFv-anti-MSLN protein constructs to RBCs was assessed by flow cytometry in a concentration dependent manner.

RBCs were isolated from peripheral blood of healthy donors by 3 cycles of centrifugation and washing with Phosphate Buffer Saline (PBS). In a 96-well plate, 150.000 RBCs were stained with the indicated primary antibodies for 30 minutes on ice, followed by staining with a fluorescently-labelled secondary antibody for 30 minutes on ice, and final detection by flow cytometry. Dots represent raw data, curves are non-linear fit of the specific signal with Hill slope=1. Mean fluorescence intensity ratio (MFI ratio) was calculated using the isotype control as reference. MFI ratio below 1.5 is considered “no binding”.

Due to the designed low affinity of the anti-CD47 scFv to CD47, the anti-CD47 scFv-anti-MSLN protein constructs showed very little binding to RBCs, as compared to a high affinity anti-CD47 mAb (see FIG. 34).

### Example 9

### Anti-CD47 Antibodies and Fragments Thereof Inducing Aggregation of Platelets (PLTs)

CD47 is expressed on platelets (PLTs), also known as thrombocytes. Aggregation of PLTs can cause thrombocytopenia, a condition when the PLT count in the blood is too low. Thrombocytopenia is especially dangerous if associated with internal bleeding. We assessed PLT aggregation in vitro by isolating PLTs from healthy donors. PLTs or platelet-rich-plasma (PRP) were isolated from 20-30 ml blood from healthy donors, drawn in a plastic syringe containing 1:10 volume CPD (citrate-phosphate-dextrose) and purified according to state-of-the-art protocols. Isolated PLTs were gently mixed with a concentration range of 1000 nM to 1 nM of an anti-CD47 scFv-anti-MSLN protein constructs or anti-CD47 scFv IgG1 fusion (2D6-059) and aggregation was assessed by an absorbance measurement at 595 nm wavelength every 15 seconds over 30 minutes at 37° C. under various shaking conditions. The percentage of aggregation was calculated with reference to changes in the absorbance of PRP and platelet-poor-plasma (PPP) incubated with PBS and according to the formula:

$$\% platelet aggregation = [ ( OD PRP - OD sample ) / ( OD PRP - OD PPP ) ] \times 100.$$

FIG. 35 illustrates that the protein construct MSL-745 and 2D6-059 according to the present invention do not cause PLT aggregation in contrast to the positive control (anti-CD47 IgG1 and anti-CD47 clone B6H12). The anti-CD47 scFv-IgG1 protein constructs thus differ from other anti-CD47 antibodies by not inducing PLT aggregation.

1. An anti-CD47 antibody or an antigen-binding fragment thereof, comprising:
a variable heavy chain complementarity determining region 1 (CDRH1) sequence selected from the amino acid sequences of SEQ ID NOs: 60;
a variable heavy chain complementarity determining region 2 (CDRH2) sequence selected from the amino acid sequences of SEQ ID NOs: 63, 65, 66, and 71;
a variable heavy chain complementarity determining region 3 (CDRH3) sequence of SEQ ID NO: 74;
a variable light chain complementarity determining region 1 (CDRL1) sequence selected from the amino acid sequences of SEQ ID NOs: 75;
a variable light chain complementarity determining region 2 (CDRL2) sequence selected from the amino acid sequences of SEQ ID NOs: 80; and
a variable light chain complementarity determining region 3 (CDRL3) sequence selected from the amino acid sequences of SEQ ID NOs: 82 and 83;
wherein the anti-CD47 antibody or antigen-binding fragment thereof is capable of blocking the interaction of CD47 with signal regulatory protein alpha (SIRPalpha).

2. (canceled)

3. The anti-CD47 antibody or antigen-binding fragment of claim 1, wherein said antibody antigen-binding fragment thereof comprises a variable heavy chain complementarity determining region 1 (CDRH1) sequence of SEQ ID NO: 60 and a variable heavy chain complementarity determining region 2 (CDRH2) sequence of SEQ ID NO: 71 and a variable heavy chain complementarity determining region 3 (CDRH3) sequence of SEQ ID NO: 74; and a variable light chain complementarity determining region 1 (CDRL1) sequence of SEQ ID NO: 75 and a variable light chain complementarity determining region 2 (CDRL2) sequence of SEQ ID NO: 80 and a variable light chain complementarity determining region 3 (CDRL3) sequence of SEQ ID NO: 83.

4. (canceled)

5. The anti-CD47 antibody or antigen-binding fragment of claim 1, wherein said antibody or antigen-binding fragment thereof comprises:
(i) a variable light chain amino acid sequence of SEQ ID NO: 2 and a variable heavy chain amino acid sequence selected from the amino acid sequences of SEQ ID NO: 31; or
(ii) a variable light chain amino acid sequence of SEQ ID NO: 3 and a variable heavy chain amino acid sequence selected from the amino acid sequences of SEQ ID NOs: 35, 36; or
(iii) a variable light chain amino acid sequence of SEQ ID NO: 4 and a variable heavy chain amino acid sequence selected from the amino acid sequences of SEQ ID NOs: 31, 35, 36;
(iv) a variable light chain amino acid sequence of SEQ ID NO: 10 and a variable heavy chain amino acid sequence of SEQ ID NO: 31; or
(v) a variable light chain amino acid sequence of SEQ ID NO: 15 and a variable heavy chain amino acid sequence of SEQ ID NO: 42; or
(vi) a variable light chain amino acid sequence of SEQ ID NO: 25 and a variable heavy chain amino acid sequence of SEQ ID NO: 42; or
(vii) a variable light chain amino acid sequence of SEQ ID NO: 27 and a variable heavy chain amino acid sequence of SEQ ID NO: 48; or
(viii) a variable light chain amino acid sequence of SEQ ID NO: 28 and a variable heavy chain amino acid sequence of SEQ ID NO: 48, preferably wherein said antibody or antigen-binding fragment thereof comprises a variable light chain amino acid sequence of SEQ ID NO: 15 and a variable heavy chain amino acid sequence of SEQ ID NO: 42.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. The anti-CD47 antibody or antigen-binding fragment of claim 1, wherein said antigen-binding fragment of the anti-CD47 antibody is a Fab or scFv domain comprising a variable light chain (VL) amino acid sequence selected from SEQ ID NOs: 2, 3, 4, 10, 15, 25, 27 or 28, and a variable heavy (VH) chain amino acid sequence selected from SEQ ID NOs: 31, 35, 36, 42, and 48, preferably comprising a variable light chain amino acid sequence of SEQ ID NO: 15 and a variable heavy chain amino acid sequence of SEQ ID NO: 42.

24. (canceled)

25. (canceled)

26. The anti-CD47 antibody or antigen-binding fragment of claim 1, wherein said anti-CD47 antibody or antigen-binding fragment has an affinity for CD47 measured by surface plasmon resonance (SPR), in the range of 100 nM to 2 μM, preferably in the range of 300 nM to 800 nM.

27. The anti-CD47 antibody or antigen-binding fragment of claim 1, wherein said anti-CD47 antibody or antigen-binding fragment has an affinity to its target CD47 which in comparison to the affinity of antibody B6H12 is lower by a factor of at least 20, preferably at least 40, more preferably at least 60.

28. The anti-CD47 antibody or antigen-binding fragment of claim 1, wherein said anti-CD47 antibody or antigen-binding fragment does not induce platelet aggregation of more than 20%, preferably at a concentration range of 10 nM to 1000 nM, more preferably at 100 nM when changes in absorbance were calculated to percentage of aggregation by reference to the absorbances of platelet rich plasma (PRP) and platelet poor plasma (PPP).

29. The anti-CD47 antibody or antigen-binding fragment of claim 1, wherein said anti-CD47 antibody or antigen-binding fragment has a K_{off} value for the binding to CD47 of about 10⁻² 1/s to 1.0 1/s, preferably of about 5.0×10⁻² 1/s to 9.0×10⁻¹ 1/s, more preferably of about 5.0×10⁻² 1/s to 6.0×10⁻¹ 1/s if measured by surface plasmon resonance (SPR).

30. An anti-CD47 antibody or antigen-binding fragment as defined in claim 1, wherein the anti-CD47 antibody or antigen-binding fragment is combined to a further functional component, wherein said combination with a further functional component is preferably a polypeptide fusion via a polypeptide linker, preferably a polypeptide linker comprising or consisting of 4 to 40 amino acids.

31. The anti-CD47 antibody or antigen-binding fragment of claim 30, wherein said further functional component is a binding domain for a tumor marker present on the surface of a tumor cell, wherein said tumor marker is Mesothelin (MSLN), B7H3, CEACAM5, CA125, EGFR, Her2 or Mucin-1.

32. (canceled)

33. (canceled)

34. (canceled)

35. (canceled)

36. A nucleic acid molecule comprising a polynucleotide encoding the anti-CD47 antibody or antigen-binding fragment thereof of claim 1.

37. A vector comprising the nucleic acid molecule of claim 36.

38. A host cell comprising the nucleic acid molecule of claim 36.

39. A host cell that expresses the anti-CD47 antibody or antigen-binding fragment thereof of claim 1.

40. (canceled)

41. (canceled)

42. A pharmaceutical composition comprising the anti-CD47 antibody or antigen-binding fragment of claim 1 and a pharmaceutically acceptable carrier;
wherein the pharmaceutical composition further comprises:
(i) an antibody or antigen-binding fragment thereof targeting Mesothelin (MSLN) such as Amatuximab, Anetumab, h7D9.v3, or BMS-986148; or
(ii) an antibody or antigen-binding fragment thereof targeting B7H3 such as Enoblituzumab, MGC018, Omburtamab or MABX-9001; or
(iii) an antibody or antigen-binding fragment thereof targeting CEACAM5 such as SAR408377, Labetuzumab, SGM-ch511, or Cergutuzumab; or
(iv) an antibody or antigen-binding fragment thereof targeting CA125 such as Abagovomab and Oregovomab; or
(v) an antibody or antigen-binding fragment thereof targeting Mucin-1 such as BTH1704, mAb-AR20.5, C595, TAB004, 1B2, HMFG1, PankoMab, KL-6, 5E5, or GGSK-1/30;
(vi) an antibody or antigen-binding fragment thereof targeting EGFR such as Cetuximab, Panitumumab, Nimotuzumab, Necitumumab, Depatuxizumab, Futuximab, Imgatuzumab, Matuzumab, GC1118, AMG595, Mab A13, MRG003, AVID100, SHR-A1307, RN765C, ABT-414, ABT-806 or ABBV-321; or
(vii) an antibody or antigen-binding fragment thereof targeting Her2 such as Pertuzumab, Trastuzumab, Margetuximab, MCLA-128, GBR 1302, RC48, DS-8201a, FS-1502, SYD985 or ARX788.

43. (canceled)

44. (canceled)

45. (canceled)

46. (canceled)

47. (canceled)

48. (canceled)

49. (canceled)

50. (canceled)

51. The anti-CD47 antibody or antigen-binding fragment of claim 1, for use in the treatment of cancer, wherein said cancer is ovarian cancer, ascites, mesothelioma, breast cancer, triple negative breast cancer, pancreatic cancer, pancreatic adenocarcinoma, non-small cell lung cancer (NSCLC), colorectal cancer, endometrial cancer, biliary extrahepatic cancer, lymphoma non-hodgkin lymphoma (NHL), Diffuse large B cell lymphoma (DLBCL) leukemia, acute myeloid leukemia (AML) and myelodysplastic syndrome (MDS).

52. (canceled)

53. (canceled)

54. A host cell comprising the vector of claim 37.

55. The pharmaceutical composition of claim 42, for use in the treatment of cancer, wherein said cancer is ovarian cancer, ascites, mesothelioma, breast cancer, triple negative breast cancer, pancreatic cancer, pancreatic adenocarcinoma, non-small cell lung cancer (NSCLC), colorectal cancer, endometrial cancer, biliary extrahepatic cancer, lymphoma non-hodgkin lymphoma (NHL), Diffuse large B cell lymphoma (DLBCL) leukemia, acute myeloid leukemia (AML) and myelodysplastic syndrome (MDS).


SEQUENCE LISTING (128 sequences)

SEQ ID NO: 1 - protein, 112 aa, Mus musculus
DIVMSQSPSSLAVSAGEKVTMSCKSSQSLLNSRTRKNYLAWYQQKPGQSPKLLIYWASTRDSGVPDRFTGSGSGTDFTLTISSVQAEDLAVYYCKQSFNLVTFGAGTKLELK

SEQ ID NO: 2 - protein, 112 aa, synthetic construct
  REGION 1..112: note = Synthetic polypeptide
DIVLTQSPSSLSASVGDRVTMSCKSSQSLLNSRTRKNYLAWYQQKPGKAPKLLIYWASTRDSGVPSRFSGSGSGTDFTLTISSLQPEDIATYYCKQSFNLVTFGGGTKLEIK

SEQ ID NO: 3 - protein, 112 aa, synthetic construct
  REGION 1..112: note = Synthetic polypeptide
DIVMTQSPSSLAVSLGERVTMSCKSSQSLLNSRTRKNYLAWYQQKPGQPPKLLIYWASTRDSGVPDRFSGSGSGTDFTLTISSLQAEDVAVYYCKQSFNLVTFGGGTKLEIK

SEQ ID NO: 4 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRDSGVPDRFSGSGSGTDFTLTISSLQAEDVAVYYCKQSFNLVTFGGGTKLEIK

SEQ ID NO: 5 - protein, 112 aa, synthetic construct
  REGION 1..112: note = Synthetic polypeptide
DIVLTQSPSSLAVSLGERVTMSCKSSQSLLYSRTRKNYLAWYQQKPGQPPKLLIYWASTRDSGVPDRFSGSGSGTDFTLTISSLQAEDVAVYYCKQSFNLVTFGGGTKLEIK

SEQ ID NO: 6 - protein, 112 aa, synthetic construct
  REGION 1..112: note = Synthetic polypeptide
DIVLTQSPSSLAVSLGERVTMSCKSSQSLLNSSTRKNYLAWYQQKPGQPPKLLIYWASTRDSGVPDRFSGSGSGTDFTLTISSLQAEDVAVYYCKQSFNLVTFGGGTKLEIK

SEQ ID NO: 7 - protein, 112 aa, synthetic construct
  REGION 1..112: note = Synthetic polypeptide
DIVLTQSPSSLAVSLGERVTMSCKSSQSLLNSRNRKNYLAWYQQKPGQPPKLLIYWASTRDSGVPDRFSGSGSGTDFTLTISSLQAEDVAVYYCKQSFNLVTFGGGTKLEIK

SEQ ID NO: 8 - protein, 112 aa, synthetic construct
  REGION 1..112: note = Synthetic polypeptide
DIVLTQSPSSLAVSLGERVTMSCKSSQSLLNSRTNKNYLAWYQQKPGQPPKLLIYWASTRDSGVPDRFSGSGSGTDFTLTISSLQAEDVAVYYCKQSFNLVTFGGGTKLEIK

SEQ ID NO: 9 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRESGVPDRFSGSGSGTDFTLTISSLQAEDVAVYYCKQSFNLVTFGGGTKLEIK

SEQ ID NO: 10 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRDSGVPDRFSGSGSGTDFTLTISSLQAEDVAVYYCQQSFNLVTFGGGTKLEIK

SEQ ID NO: 11 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRDSGVPDRFSGSGSGTDFTLTISSLQAEDVAVYYCKQSFSLVTFGGGTKLEIK

SEQ ID NO: 12 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRDSGVPDRFSGSGSGTDFTLTISSLQAEDVAVYYCKQSFNTVTFGGGTKLEIK

SEQ ID NO: 13 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRESGVPDRFSGSGSGTDFTLTISSLQAEDVAVYYCKQSFNTVTFGGGTKLEIK

SEQ ID NO: 14 - protein, 112 aa, synthetic construct
  REGION 1..112: note = Synthetic polypeptide
DIVLTQSPSSLAVSLGERVTMSCKSSQSLLYSRTRKNYLAWYQQKPGQPPKLLIYWASTRDSGVPDRFSGSGSGTDFTLTISSLQAEDVAVYYCQQSFNLVTFGGGTKLEIK

SEQ ID NO: 15 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRDSGVPDRFSGSGSGTDFTLTISSLQAEDVAVYYCQQSFNLVTFGCGTKLEIK

SEQ ID NO: 16 - protein, 112 aa, synthetic construct
  REGION 1..112: note = Synthetic polypeptide
DIVLTQSPSSLAVSLGERVTMSCKSSQSLLNSRTNKNYLAWYQQKPGQPPKLLIYWASTRDSGVPDRFSGSGSGTDFTLTISSLQAEDVAVYYCQQSFNLVTFGCGTKLEIK

SEQ ID NO: 17 - protein, 112 aa, synthetic construct
  REGION 1..112: note = Synthetic polypeptide
DIVMTQSPDSLAVSLGERATMSCKSSQSLLNSRTRKNYLAWYQQKPGQPPKLLIYWASTRDSGVPDRFSGSGSGTDFTLTISSLQAEDVAVYYCQQSFNLVTFGGGTKLEIK

SEQ ID NO: 18 - protein, 112 aa, synthetic construct
  REGION 1..112: note = Synthetic polypeptide
DIVMTQSPDSLAVSLGERATMSCKSSQSLLNSRTRKNYLAWYQQKPGQPPKLLIYWASTRDSGVPDRFSGSGSGTDFTLTISSLQAEDVAVYYCQQSFNLVTFGCGTKLEIK

SEQ ID NO: 19 - protein, 112 aa, synthetic construct
  REGION 1..112: note = Synthetic polypeptide
DIVMTQSPDSLAVSLGERVTMSCKSSQSLLNSRTRKNYLAWYQQKPGQPPKLLIYWASTRDSGVPDRFSGSGSGTDFTLTISSLQAEDVAVYYCQQSFNLVTFGGGTKLEIK

SEQ ID NO: 20 - protein, 112 aa, synthetic construct
  REGION 1..112: note = Synthetic polypeptide
DIVMTQSPDSLAVSLGERVTMSCKSSQSLLNSRTRKNYLAWYQQKPGQPPKLLIYWASTRDSGVPDRFSGSGSGTDFTLTISSLQAEDVAVYYCQQSFNLVTFGCGTKLEIK

SEQ ID NO: 21 - protein, 112 aa, synthetic construct
  REGION 1..112: note = Synthetic polypeptide
DIVLTQSPSSLAVSLGERATMSCKSSQSLLNSRTRKNYLAWYQQKPGQPPKLLIYWASTRDSGVPDRFSGSGSGTDFTLTISSLQAEDVAVYYCQQSFNLVTFGGGTKLEIK

SEQ ID NO: 22 - protein, 112 aa, synthetic construct
  REGION 1..112: note = Synthetic polypeptide
DIVLTQSPSSLAVSLGERATMSCKSSQSLLNSRTRKNYLAWYQQKPGQPPKLLIYWASTRDSGVPDRFSGSGSGTDFTLTISSLQAEDVAVYYCQQSFNLVTFGCGTKLEIK

SEQ ID NO: 23 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRESGVPDRFSGSGSGTDFTLTISSLQAEDVAVYYCQQSFNLVTFGGGTKLEIK

SEQ ID NO: 24 - protein, 112 aa, synthetic construct
  REGION 1..112: note = Synthetic polypeptide
DIVLTQSPSSLAVSLGERVTMSCKSSQSLLNSRTRKNYLAWYQQKPGQPPKLLIYWASTRESGVPDRFSGSGSGTDFTLTISSLQAEDVAVYYCQQSFNLVTFGCGTKLEIK

SEQ ID NO: 25 - protein, 113 aa, synthetic construct
  REGION 1..113: note = Synthetic polypeptide
DIVLTQSPSSLAVSLGERVTMSCKSSQSLLNSRTRKNYLAWYQQKPGQPPKLLIYWASTRDSGVPDRFSGSGSGTDFTLTISSLQAEDVAVYYCQQSFNLVTFGCGTKLEIKR

SEQ ID NO: 26 - protein, 113 aa, synthetic construct
  REGION 1..113: note = Synthetic polypeptide
DIVMTQSPDSLAVSLGERATMSCKSSQSLLNSRTRKNYLAWYQQKPGQPPKLLIYWASTRDSGVPDRFSGSGSGTDFTLTISSLQAEDVAVYYCQQSFNLVTFGCGTKLEIKR

SEQ ID NO: 27 - protein, 112 aa, synthetic construct
  REGION 1..112: note = Synthetic polypeptide
DIQMTQSPSSLSASVGDRVTITCKSSQSLLNSRTRKNYLAWYQQKPGKAPKLLIYWASTRDSGVPSRFSGSGSGTDFTLTISSLQPEDFATYYCKQSFNLVTFGQGTKVEIK

SEQ ID NO: 28 - protein, 112 aa, synthetic construct
  REGION 1..112: note = Synthetic polypeptide
DIVMTQSPSSLSASVGDRVTITCKSSQSLLNSRTRKNYLAWYQQKPGKAPKLLIYWASTRDSGVPDRFSGSGSGTDFTLTISSLQPEDFATYYCKQSFNLVTFGQGTKVEIK

SEQ ID NO: 29 - protein, 119 aa, synthetic construct
  REGION 1..119: note = Synthetic polypeptide
QVQLQQSGPELVKPGTSVTLSCKASGYTFPSYDINWMKQRPGQGLEWIGWIYPRDGSPKYNEKFKGKATLTVDTSSSTAYMELHSLTSEDSAVYFCARSVYYGTQFAYWGQGTLVTVSA

SEQ ID NO: 30 - protein, 119 aa, synthetic construct
  REGION 1..119: note = Synthetic polypeptide
QVELVQSGAEVKKPGSSVKVSCKASGGTFPSYDINWVRQAPGQGLEWMGWIYPRDGSPKYNEKFKGRVTITADESTSTAYMELSSLRSEDTAVYYCARSVYYGTQFAYWGQGTLVTVSS

SEQ ID NO: 31 - protein, 119 aa, synthetic construct
  REGION 1..119: note = Synthetic polypeptide
QVELVQSGPEVKKPGSSVKVSCKASGYTFPSYDINWVRQRPGQGLEWMGWIYPRDGSPKYNEKFKGRVTITADTSTSTAYMELHSLRSEDTAVYYCARSVYYGTQFAYWGQGTLVTVSA

SEQ ID NO: 32 - protein, 119 aa, synthetic construct
  REGION 1..119: note = Synthetic polypeptide
QVELVQSGPEVKKPGSSVKVSCKASGYTFPSYAINWVRQRPGQGLEWMGWIYPRDGSPKYNEKFKGRVTITADTSTSTAYMELHSLRSEDTAVYYCARSVYYGTQFAYWGQGTLVTVSA

SEQ ID NO: 33 - protein, 119 aa, synthetic construct
  REGION 1..119: note = Synthetic polypeptide
QVELVQSGPEVKKPGSSVKVSCKASGYTFPSYDISWVRQRPGQGLEWMGWIYPRDGSPKYNEKFKGRVTITADTSTSTAYMELHSLRSEDTAVYYCARSVYYGTQFAYWGQGTLVTVSA

SEQ ID NO: 34 - protein, 119 aa, synthetic construct
  REGION 1..119: note = Synthetic polypeptide
QVELVQSGPEVKKPGSSVKVSCKASGYTFPSYDINWVRQRPGQGLEWMGGIYPRDGSPKYNEKFKGRVTITADTSTSTAYMELHSLRSEDTAVYYCARSVYYGTQFAYWGQGTLVTVSA

SEQ ID NO: 35 - protein, 119 aa, synthetic construct
  REGION 1..119: note = Synthetic polypeptide
QVELVQSGPEVKKPGSSVKVSCKASGYTFPSYDINWVRQRPGQGLEWMGWIIPRDGSPKYNEKFKGRVTITADTSTSTAYMELHSLRSEDTAVYYCARSVYYGTQFAYWGQGTLVTVSA

SEQ ID NO: 36 - protein, 119 aa, synthetic construct
  REGION 1..119: note = Synthetic polypeptide
QVELVQSGPEVKKPGSSVKVSCKASGYTFPSYDINWVRQRPGQGLEWMGWIYPRFGSPKYNEKFKGRVTITADTSTSTAYMELHSLRSEDTAVYYCARSVYYGTQFAYWGQGTLVTVSA

SEQ ID NO: 37 - protein, 119 aa, synthetic construct
  REGION 1..119: note = Synthetic polypeptide
QVELVQSGPEVKKPGSSVKVSCKASGYTFPSYDINWVRQRPGQGLEWMGWIYPRDGSAKYNEKFKGRVTITADTSTSTAYMELHSLRSEDTAVYYCARSVYYGTQFAYWGQGTLVTVSA

SEQ ID NO: 38 - protein, 119 aa, synthetic construct
  REGION 1..119: note = Synthetic polypeptide
QVELVQSGPEVKKPGSSVKVSCKASGYTFPSYDINWVRQRPGQGLEWMGWIYPRDGSPNYNEKFKGRVTITADTSTSTAYMELHSLRSEDTAVYYCARSVYYGTQFAYWGQGTLVTVSA

SEQ ID NO: 39 - protein, 119 aa, synthetic construct
  REGION 1..119: note = Synthetic polypeptide
QVELVQSGPEVKKPGSSVKVSCKASGYTFPSYDINWVRQRPGQGLEWMGWIYPRDGSPKYNQKFKGRVTITADTSTSTAYMELHSLRSEDTAVYYCARSVYYGTQFAYWGQGTLVTVSA

SEQ ID NO: 40 - protein, 119 aa, synthetic construct
  REGION 1..119: note = Synthetic polypeptide
QVELVQSGPEVKKPGSSVKVSCKASGYTFPSYDINWVRQRPGQGLEWMGWIYPRSGSPKYNEKFKGRVTITADTSTSTAYMELHSLRSEDTAVYYCARSVYYGTQFAYWGQGTLVTVSA

SEQ ID NO: 41 - protein, 119 aa, synthetic construct
  REGION 1..119: note = Synthetic polypeptide
QVELVQSGPEVKKPGSSVKVSCKASGYTFPSYDINWVRQRPGQGLEWMGGIYPRSGSPKYNEKFKGRVTITADTSTSTAYMELHSLRSEDTAVYYCARSVYYGTQFAYWGQGTLVTVSA

SEQ ID NO: 42 - protein, 119 aa, synthetic construct
  REGION 1..119: note = Synthetic polypeptide
QVELVQSGPEVKKPGSSVKVSCKASGYTFPSYDINWVRQRPGQCLEWMGGIYPRSGSPKYNEKFKGRVTITADTSTSTAYMELHSLRSEDTAVYYCARSVYYGTQFAYWGQGTLVTVSA

SEQ ID NO: 43 - protein, 119 aa, synthetic construct
  REGION 1..119: note = Synthetic polypeptide
QVELVQSGPEVKKPGSSVKVSCKASGYTFPSYDINWVRQRPGQCLEWMGGIYPRSGSPKYNQKFKGRVTITADTSTSTAYMELHSLRSEDTAVYYCARSVYYGTQFAYWGQGTLVTVSA

SEQ ID NO: 44 - protein, 119 aa, synthetic construct
  REGION 1..119: note = Synthetic polypeptide
QVELVQSGPEVKKPGSSVKVSCKASGYTFPSYDINWVRQRPGQCLEWMGGIYPRSGSAKYNEKFKGRVTITADTSTSTAYMELHSLRSEDTAVYYCARSVYYGTQFAYWGQGTLVTVSA

SEQ ID NO: 45 - protein, 119 aa, synthetic construct
  REGION 1..119: note = Synthetic polypeptide
QVELVQSGPEVKKPGSSVKVSCKASGYTFPSYDINWVRQRPGQGLEWMGGIYPRSGSPKYNEKFKGRVTITADTSTSTAYMELSSLRSEDTAVYYCARSVYYGTQFAYWGQGTLVTVSA

SEQ ID NO: 46 - protein, 119 aa, synthetic construct
  REGION 1..119: note = Synthetic polypeptide
QVELVQSGPEVKKPGSSVKVSCKASGYTFPSYDINWVRQRPGQCLEWMGGIYPRSGSPKYNEKFKGRVTITADTSTSTAYMELSSLRSEDTAVYYCARSVYYGTQFAYWGQGTLVTVSA

SEQ ID NO: 47 - protein, 119 aa, synthetic construct
  REGION 1..119: note = Synthetic polypeptide
QVQLVESGGGLVQPGGSLRLSCAASGYTFTSYDINWVRQAPGKGLEWVGWIYPRDGSPKYNEKFKGRFTISADTSKNTAYLQMNSLRAEDTAVYYCARSVYYGTQFAYWGQGTLVTVSS

SEQ ID NO: 48 - protein, 119 aa, synthetic construct
  REGION 1..119: note = Synthetic polypeptide
QVQLVESGGGLVQPGGSLRLSCAASGYTFPSYDINWVRQAPGKGLEWVGWIYPRDGSPKYNEKFKGRFTISADTSKNTAYLQMNSLRAEDTAVYYCARSVYYGTQFAYWGQGTLVTVSS

SEQ ID NO: 49 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 50 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 51 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 15 aa, Homo sapiens
EPKSCDKTHTCPPCP

SEQ ID NO: 53 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 55 - protein, 110 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 56 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 12 aa, Homo sapiens
ESKYGPPCPSCP

SEQ ID NO: 58 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 59 - protein, 107 aa, Homo sapiens [sequence omitted]

SEQ ID NO: 60 - protein, 5 aa, synthetic construct
  REGION 1..5: note = Synthetic polypeptide
SYDIN

SEQ ID NO: 61 - protein, 5 aa, synthetic construct
  REGION 1..5: note = Synthetic polypeptide
SYAIN

SEQ ID NO: 62 - protein, 5 aa, synthetic construct
  REGION 1..5: note = Synthetic polypeptide
SYDIS

SEQ ID NO: 63 - protein, 17 aa, synthetic construct
  REGION 1..17: note = Synthetic polypeptide
WIYPRDGSPKYNEKFKG

SEQ ID NO: 64 - protein, 17 aa, synthetic construct
  REGION 1..17: note = Synthetic polypeptide
GIYPRDGSPKYNEKFKG

SEQ ID NO: 65 - protein, 17 aa, synthetic construct
  REGION 1..17: note = Synthetic polypeptide
WIIPRDGSPKYNEKFKG

SEQ ID NO: 66 - protein, 17 aa, synthetic construct
  REGION 1..17: note = Synthetic polypeptide
WIYPRFGSPKYNEKFKG

SEQ ID NO: 67 - protein, 17 aa, synthetic construct
  REGION 1..17: note = Synthetic polypeptide
WIYPRDGSAKYNEKFKG

SEQ ID NO: 68 - protein, 17 aa, synthetic construct
  REGION 1..17: note = Synthetic polypeptide
WIYPRDGSPNYNEKFKG

SEQ ID NO: 69 - protein, 17 aa, synthetic construct
  REGION 1..17: note = Synthetic polypeptide
WIYPRDGSPKYNQKFKG

SEQ ID NO: 70 - protein, 17 aa, synthetic construct
  REGION 1..17: note = Synthetic polypeptide
WIYPRSGSPKYNEKFKG

SEQ ID NO: 71 - protein, 17 aa, synthetic construct
  REGION 1..17: note = Synthetic polypeptide
GIYPRSGSPKYNEKFKG

SEQ ID NO: 72 - protein, 17 aa, synthetic construct
  REGION 1..17: note = Synthetic polypeptide
GIYPRSGSPKYNQKFKG

SEQ ID NO: 73 - protein, 17 aa, synthetic construct
  REGION 1..17: note = Synthetic polypeptide
GIYPRSGSAKYNEKFKG

SEQ ID NO: 74 - protein, 10 aa, synthetic construct
  REGION 1..10: note = Synthetic polypeptide
SVYYGTQFAY

SEQ ID NO: 75 - protein, 17 aa, synthetic construct
  REGION 1..17: note = Synthetic polypeptide
KSSQSLLNSRTRKNYLA

SEQ ID NO: 76 - protein, 17 aa, synthetic construct
  REGION 1..17: note = Synthetic polypeptide
KSSQSLLYSRTRKNYLA

SEQ ID NO: 77 - protein, 17 aa, synthetic construct
  REGION 1..17: note = Synthetic polypeptide
KSSQSLLNSSTRKNYLA

SEQ ID NO: 78 - protein, 17 aa, synthetic construct
  REGION 1..17: note = Synthetic polypeptide
KSSQSLLNSRNRKNYLA

SEQ ID NO: 79 - protein, 17 aa, synthetic construct
  REGION 1..17: note = Synthetic polypeptide
KSSQSLLNSRTNKNYLA

SEQ ID NO: 80 - protein, 7 aa, synthetic construct
  REGION 1..7: note = Synthetic polypeptide
WASTRDS

SEQ ID NO: 81 - protein, 7 aa, synthetic construct
  REGION 1..7: note = Synthetic polypeptide
WASTRES

SEQ ID NO: 82 - protein, 8 aa, synthetic construct
  REGION 1..8: note = Synthetic polypeptide
KQSFNLVT

SEQ ID NO: 83 - protein, 8 aa, synthetic construct
  REGION 1..8: note = Synthetic polypeptide
QQSFNLVT

SEQ ID NO: 84 - protein, 8 aa, synthetic construct
  REGION 1..8: note = Synthetic polypeptide
KQSFSLVT

SEQ ID NO: 85 - protein, 8 aa, synthetic construct
  REGION 1..8: note = Synthetic polypeptide
KQSFNTVT

SEQ ID NO: 86 - protein, 5 aa, synthetic construct
  REGION 1..5: note = Synthetic polypeptide
GGGGS

SEQ ID NO: 87 - protein, 4 aa, synthetic construct
  REGION 1..4: note = Synthetic polypeptide
GGGS

SEQ ID NO: 88 - protein, 5 aa, synthetic construct
  REGION 1..5: note = Synthetic polypeptide
GGGGG

SEQ ID NO: 89 - protein, 4 aa, synthetic construct
  REGION 1..4: note = Synthetic polypeptide
GGGG

SEQ ID NO: 90-91 - skipped (2 entries)

SEQ ID NO: 92 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 93 - protein, 292 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 94 - protein, 245 aa, Homo sapiens
KGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRNN

SEQ ID NO: 95 - protein, 311 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLN

SEQ ID NO: 96 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 681 aa, Homo sapiens
MALPTARPLLGSCGTPALGSLLFLLFSLGWVQPSRTLAGETGQEAAPLDGVMALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 99 - protein, 651 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLAQEALSGTPCLLGPGPVLTVLALLLASTLA

SEQ ID NO: 100 - protein, 656 aa, Homo sapiens [sequence omitted]

SEQ ID NO: 101 - protein, 621 aa, Homo sapiens
MALPTARPLLGSCGTPALGSLLFLLFSLGWVQPSRTLAGETGQAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 102 - protein, 107 aa, synthetic construct
  REGION 1..107: note = Synthetic polypeptide
DIQMTQSPSSLSASVGDRVTITCKASQDVGTAVAWYQQKPGKAPKALIYYASTRHTGVPDRFSGSGSGTDFTLTISSLQPEDFATYYCQQYSSYPLTFGQGTKVEIK

SEQ ID NO: 103 - protein, 120 aa, synthetic construct
  REGION 1..120: note = Synthetic polypeptide
QVQLVESGGGLVQPGGSLRLSCAASGYTFTSYWMHWMNQAPGKGLEWVGLIHPNSGSANYNEKFKNRFTISADKSSNTAYLQMNSLRAEDTAVYYCARITTVVAPYFDYWGQGTTVTVSS

SEQ ID NO: 104 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 105 - protein, 316 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 106 - protein, 493 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPGPASSAVPLSPAHPPHGSMCWSHWFSRGL

SEQ ID NO: 107 - protein, 533 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEGKDTWA

SEQ ID NO: 108 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 109 - protein, 701 aa, Homo sapiens [sequence omitted]

SEQ ID NO: 110 - protein, 14502 aa, Homo sapiens
MLKPSGLPGSSSPTRSLMTGSRSTKATPEMDSGLTGATLSPKTSTGAIVVTEHTLPFTSPDKTLASPTSSVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 111 - protein, 5 aa, synthetic construct
  REGION 1..5: note = Synthetic polypeptide
SYWMH

SEQ ID NO: 112 - protein, 17 aa, synthetic construct
  REGION 1..17: note = Synthetic polypeptide
LIHPNSGSANYNEKFKN

SEQ ID NO: 113 - protein, 11 aa, synthetic construct
  REGION 1..11: note = Synthetic polypeptide
ITTVVAPYFDY

SEQ ID NO: 114 - protein, 11 aa, synthetic construct
  REGION 1..11: note = Synthetic polypeptide
KASQDVGTAVA

SEQ ID NO: 115 - protein, 7 aa, synthetic construct
  REGION 1..7: note = Synthetic polypeptide
YASTRHT

SEQ ID NO: 116 - protein, 9 aa, synthetic construct
  REGION 1..9: note = Synthetic polypeptide
QQYSSYPLT

SEQ ID NO: 117 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 118 - protein, 255 aa, Homo sapiens
MTPGTQSPFFLLLLLTVLTVVTGSGHASSTPGGEKETSATQRSSVPSSTEKNAFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 119 - protein, 5 aa, synthetic construct
SYWMS

SEQ ID NO: 120 - protein, 17 aa, synthetic construct
DIKHDGSFTNYAPSLKN

SEQ ID NO: 121 - protein, 10 aa, synthetic construct
DQRYYSLSGY

SEQ ID NO: 122 - protein, 11 aa, synthetic construct
QASQDIGNWLA

SEQ ID NO: 123 - protein, 7 aa, synthetic construct
SATSLAD

SEQ ID NO: 124 - protein, 9 aa, synthetic construct
LQRYSNPWT

SEQ ID NO: 125 - protein, 119 aa, Rattus norvegicus
EVQLVESGGGLVQPGGSLRLSCAASGFTFSSYWMSWVRQAPGKGLEWVADIKHDGSFTNYAPSLKNRFTISRDNAKNTLYLQMNSLRAEDTAVYYCARDQRYYSLSGYWGQGTLVTVSS

SEQ ID NO: 126 - protein, 107 aa, Rattus norvegicus
DIQMTQSPSSLSASVGDRVTITCQASQDIGNWLAWYQQKPGKAPKLLIYSATSLADGVPSRFSGSGSGTDFTLTISSLQPEDFATYYCLQRYSNPWTFGGGTKVEIK

SEQ ID NO: 127 - protein, 5 aa, synthetic construct
DYWMS

SEQ ID NO: 128 - protein, 119 aa, synthetic construct
EVQLVESGGGLVQPGGSLRLSCAASGFTFSDYWMSWVRQAPGKGLEWVADIKHDGSFTNYAPSLKNRFTISRDNAKNTLYLQMNSLRAEDTAVYYCARDQRYYSLSGYWGQGTLVTVSS